(19) United States
(12) Patent Application Publication
(10) Pub. No.: US 2026/0297689 A1
(43) Pub. Date: Oct. 1, 2026

(54) METHODS AND COMPOSITIONS FOR DETECTING MICROBIAL GROWTH IN BUILT ENVIRONMENTS

(71) Applicants: Karen DANNEMILLER, Columbus, OH (US); Ashleigh BOPE, Columbus, OH (US); Neeraja BALASUBRAHMANIAM, Columbus, OH (US); Ohio State Innovation Foundation, Columbus, OH (US)

(72) Inventors: Karen DANNEMILLER, Columbus, OH (US); Ashleigh BOPE, Reynoldsburg, OH (US); Neeraja BALASUBRAHMANIAM, Columbus, OH (US); Bridget Emily HEGARTY, University Heights, OH (US); Jonathan Christopher KING, Columbus, OH (US)

(73) Assignee: Ohio State Innovation Foundation, Columbus, OH (US)

(21) Appl. No.: 19/476,038

(22) PCT Filed: Apr. 17, 2024
(86) PCT No.: PCT/US2024/025034

§ 371 Date: Oct. 16, 2025

Related U.S. Application Data
(60) Provisional application No. 63/496,481, filed on Apr. 17, 2023.

(51) Int. Cl. C12Q 1/6895; A61L 2/24; C12Q 1/689

(52) U.S. Cl. CPC C12Q 1/6895; A61L 2/24; C12Q 1/689; A61L 2202/14; C12Q 2600/158

(57) ABSTRACT

The present invention provides methods to inhibit or reduce microbial growth in a built environment, with a damp environment. Also disclosed herein is a kit used to identify and quantify the expression of one or more gene(s) or one or more product(s) thereof, of one or more microbe(s) in at least one sample obtained from a built environment.

### CROSS-REFERENCE TO RELATED APPLICATIONS

This application claims priority to, and the benefit of, U.S. Provisional Patent Application Ser. No. 63/496,481, filed Apr. 17, 2023, the disclosure of which is expressly incorporated herein by reference in its entirety.

### GOVERNMENT SUPPORT CLAUSE

This invention was made with government support under grant/contract no. 1942501 awarded by the National Science Foundation. The government has certain rights in the invention.

### BACKGROUND

Exposure to mold in housing costs an estimated $22.4 billion per year in the United States alone. Such exposure can affect the health of everyone, but especially the 8% of the United States population that has asthma. However, the precise causal mechanism is unknown.

Many believe that some particular microbial species are associated with these negative health effects. Especially, homes with excess moisture and/or with visible mold growth have an established negative impact on human health. Water damaged and moldy homes are consistently associated with asthma, respiratory and allergic health outcomes, in both children and adults. These health effects disproportionately affect low-income and minority communities, including those with substandard housing conditions. These residents are often renters and/or may not have the resources for appropriate remediation of mold-damaged homes. Owners of a moldy or flooded home generally contact a remediation agency. After remediation, most customers want assurance through measurement that the cleanup is complete, and the home is free of latent microbial growth.

However, there is currently no validated quantitative test in use that is more associated with health effects from damp homes than subjective measures of mold such as visual inspection and detection of moldy odor compared to any available quantitative mold measure. Repeated evidence suggests that it is microbial growth occurring in response to indoor dampness that mediates the link between exposures and health effects. Other traditional methods to measure indoor mold including using counts of microbial spores and fungal indicators like glucans and ergosterol, have not shown consistent associations with health effects. Next generation DNA sequencing-based tools using sequence analysis of microbes have not yet been able to identify specific species as a consistent microbial signature of dampness. Solely analyzing species composition changes in response to moisture is not sufficient to quantify microbial growth due to the influence of sampling site. This technological gap reflects a fundamental lack of scientific understanding of microbial activity and function within a flooded home and how these changes are associated with health outcomes.

There is a need for tools that improve building diagnosis and clearance certification for mold industry practitioners.

### SUMMARY

The present invention relates to methods and kits and the use thereof to detect microbial growth in indoor environments that may contribute to poor indoor air quality and poor health outcomes. The present disclosure addresses at least a portion of the problems described above through the use of the method of detection and using the inventive kit for detection of microbial growth in a built environment.

In one aspect, the present invention provides a method of inhibiting or reducing microbial growth in a built environment in at least one sample collected from the built environment, wherein the microbial growth is associated with damp conditions. In some embodiments, the built environment is selected from the group comprising: a laboratory, a hospital, a manufacturing plant, an airport, an airplane, a school, an office, a vehicle, an apartment complex, a dormitory, a barrack, a prison, a spacecraft, or a home. In some embodiments, the sample is a dust sample, a surface sample, an air sample, a water sample, and/or a combination of environmental samples. In some embodiments, the microbial growth is identified by detecting one or more gene(s) and product(s) thereof which are associated with a bio-process of sporulation or other growth processes in one or more microbe(s) in at least one sample collected from the built environment. In some embodiments, the one or more microbe(s) is a bacterium and/or fungus or protozoa. In some embodiments, the fungus is mold. In some embodiments, the fungus is Aspergillus, Neurospora, Myxococcus, Saccharomyces, or Penicillium or any other fungal taxa. In some embodiments, the one or more gene(s) or product(s) thereof can be related to morphological change, secondary metabolism, stress response, mitochondria or any process associated with microbial growth and are selected from genes in Table 2. In some embodiments, the one or more gene(s) or product(s) thereof can be selected from a group comprising ALTA7, atfB, catA, hsp30, nuo-49, rodA, wA, arp1, arp2, gel2, gna-3, mpg1, mtlD, pkaR, tpsA, velB, vosA, wetA, Canx homolog, tpiA, camA, crzA, ecm33, hex1, bimG, mdm10, cap2, catB, chsA, gpaA, nudA, rhoC, steA, candA-C, candA-N, ccg-8, dop1, flbA, fluG, laeA, nudG, sidH, chsC, grrA, hymA, or sidC (GenBank Accession number—PRJNA1072816). In some embodiments, the one or more genes can be bacterial or fungal. In some embodiments, at least one fungal gene and/or at least one bacterial gene is selected. In some embodiments, the one or more gene(s) and product(s) thereof are associated with a functional annotation related to fungal (mold) growth and sporulation, measured using Gene Ontology or GO.

In some embodiments, the one or more gene(s) or product(s) thereof are measured by identifying a protein, a metabolite, a volatile organic compound, a chemical product, or a nucleic acid in the sample. In some embodiments the nucleic acid is DNA and/or RNA. In some embodiments, the method of identifying the one or more gene(s) or product(s) thereof comprises quantitative polymerase chain reaction (qPCR), mass spectrometry, liquid chromatography, lateral flow chromatography, colorimetric dye, fluorescent dye, Biuret, Bradford, bicinchoninic, Folin-Lowry, Kjeldahl, antibody binding, ultraviolet light absorbance, gel electrophoresis, capillary electrophoresis, diphenylamine, polymerase chain reaction, RFLP analysis, protein detection methods and/or a combination thereof. In some embodiments, the expression of one or more gene(s) is quantified by quantitative polymerase chain reaction (qPCR). In some embodiments, the one or more gene(s) or product(s) thereof is identified by detecting and/or quantifying the one or more gene(s) or product(s) using RNA Sequencing (RNA-Seq). In some embodiments, the product(s) are identified by detecting and/or quantifying the product(s) by lateral flow chromatography.

In some embodiments, the ALTA7, atfB, catA, hsp30, nuo-49, rodA, wA, arp1, arp2, gel2, gna-3, mpg1, mtlD, pkaR, tpsA, velB, vosA, wetA, Canx homolog, tpiA, camA, crzA, ecm33, hex1, bimG, mdm10, cap2, catB, chsA, gpaA, nudA, rhoC, steA, candA-C, candA-N, ccg-8, dop1, flbA, fluG, laeA, nudG, sidH, chsC, grrA, hymA, or sidC are found in multiple taxa in the fungal kingdom, including Aspergillus nidulans, Neurospora crassa, Myxococcus xanthus, Saccharomyces Cerevisiae and other fungal taxa. In some embodiments, ALTA7, atfB, catA, hsp30, nuo-49, rodA, wA, arp1, arp2, gel2, gna-3, mpg1, mtlD, pkaR, tpsA, velB, vosA, wetA, Canx homolog, tpiA, camA, crzA, ecm33, hex1, bimG, mdm10, cap2, catB, chsA, gpaA, nudA, rhoC, steA, candA-C, candA-N, ccg-8, dop1, flbA, fluG, laeA, nudG, sidH, chsC, grrA, hymA, or sidC are associated with a fungal growth process such as hyphal extension, sporulation and/or a combination thereof.

In further aspects the present invention provides a method of inhibiting or reducing microbial growth by treating the built environment with a microbial growth inhibitor, once, daily for at least a week, using a microbial growth inhibition technique. In some embodiments, the microbial growth inhibitor can be a dehumidifier, an exhaust fan, an antimicrobial compound, a hydrophobic paint, or a combination thereof.

In a further aspect, the present invention provides a kit for the detection of microbial growth in a built environment comprising identifying and quantifying expression of one or more gene(s) or product(s) thereof are selected from genes in Table 2. In some embodiments, the one or more gene(s) or product(s) thereof can be selected from a group comprising ALTA7, atfB, catA, hsp30, nuo-49, rodA, wA, arp1, arp2, gel2, gna-3, mpg1, mtlD, pkaR, tpsA, velB, vosA, wetA, Canx homolog, tpiA, camA, crzA, ecm33, hex1, bimG, mdm10, cap2, catB, chsA, gpaA, nudA, rhoC, steA, candA-C, candA-N, ccg-8, dop1, flbA, fluG, laeA, nudG, sidH, chsC, grrA, hymA, or sidC, in one or more microbe(s) in at least one sample obtained from a built environment wherein the expression of one or more gene(s) or quantity of product(s) thereof is measured in a nucleic acid comprising of DNA and/or RNA or in a whole protein lysate obtained from the at least one sample.

In further aspects, the kit comprises a sample collection device. In some embodiments the sample collection device is selected from a group comprising of a swab, a brush, tubes with lids, a pair of forceps, a vacuum cleaner with a collection bag, a canister, a zip-top bag, or a combination thereof. In some embodiments, the kit comprises a glass chamber, salt solution or distilled water to maintain relative humidity, a dew point water activity meter, nucleic acid extraction reagents, one or more control sample(s), a nucleic acid detection probe, DNA or RNA polymerase and a thermocycler. In some embodiments, the nucleic acid detection probe is a pair of forward and reverse primers. In some embodiments, the kit comprises of protein extraction reagents, a protein detection probe, and a lateral flow chromatography device. In some embodiments, the lateral flow chromatography device comprises of a protein lysate loading well, protein detection probe bound to a nitrocellulose membrane and a sample running buffer. In some embodiments, the protein detection probe is an antibody. In further embodiments, the kit comprises a sample resuspension buffer, a lysis buffer, a wash buffer, a phenol, and chloroform for extraction of the nucleic acid or proteins.

As disclosed herein, the expression of the one or more gene(s) is identified and quantified by quantitative polymerase chain reaction (qPCR), and product(s) thereof is detected by lateral flow chromatography. In some embodiments, the qPCR results can be read via a smart phone-based application, i.e., and the quantity of the product(s) thereof detected on the lateral flow chromatography device can be quantified via a smart phone-based application. In further embodiments, the expression of the one or more gene(s) or quantity of the product(s) thereof is compared to a control with a threshold value, database value, normalized value, relative value, validated value, or a combination thereof. In some embodiments, an increase in the expression of the one or more gene(s) or product(s) thereof compared to the control indicates microbial growth and a decrease in the expression of the one or more gene(s) or the quantity of product(s) thereof compared to the control indicates a lack of microbial growth. In some embodiments, any one gene is used to normalize the expression of the one or more gene(s) or the quantity of product(s) thereof.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF FIGURES

FIG. 1 shows an overview of methods.

FIG. 2 shows locations of participating homes that donated dust to the study. Dust from 9 homes were collected and used for experiments, sequencing, and downstream bioinformatics analysis. Dust from one home (located in TX, marked ‘X’) was collected but excluded from sequencing due to low quality of extracted RNA.

FIGS. 3A-3D show PCA plots of gene expression. FIG. 3A shows a PCA plot of gene expression in house dust grouped by ERH. PCoA of the relative abundance of fungal species in house dust grouped by ERH is shown in FIG. 3B. FIG. 3C shows a PCA plot of gene expression by site and FIG. 3D shows a PCoA of the relative abundance of fungal species by site. Color-intensity of samples are specific to ERH or site, and shapes are specific to ERH. Overlap between samples indicates greater similarity based on between-sample distance. A 95% confidence ellipse was added for each ERH condition in FIGS. 3A and 3B.

FIG. 4 shows a metabolic pathway map of fungal genes upregulated at 100% ERH when compared to 50% ERH.

FIG. 5 shows a bubble plot of representative GO terms associated with fungal growth in all ERH comparisons. Bubble sizes represent the number of upregulated genes within a GO category for a specific ERH comparison. Bubble color intensity values are based on the significance (−log 10 (FDR)) of the GO term with darker intensity representing higher significance of GO enrichment. GO terms having similar functions were grouped into broader categories. Bars next to GO terms indicate the four broader categories: Morphological, Stress response, Mitochondria and Secondary metabolism.

FIG. 6 shows a heatmap of TMM-normalized CPM expression values of fungal target genes upregulated at 100% ERH condition (top group) and upregulated at both 100% and 85% ERH (bottom group). Darker color intensities represent higher gene expression values. The bottom of the heatmap shows state locations ordered from west to east. Genes are ordered alphabetically.

FIGS. 7A-7C show bubble plots of log 2FC values for target genes in each of the three upregulated fungal gene groups. FIG. 7A shows genes upregulated at 100% ERH. FIG. 7B shows genes upregulated at 100% and 85% ERH and FIG. 7C shows genes upregulated at 85% along with their broad functional categories. Bubble color intensities represent functional categories, and the bubble size represents the magnitude of the log 2FC value. The log 2FC values of genes upregulated at both 100% and 85% ERH are based on the 100% vs 50% comparison.

FIGS. 8A-8B show from top to bottom: contig count for clusters formed by Trinity, CD-HIT-EST clusters, contigs with a Swiss-Prot database annotation, contigs with a gene ontology mapping, and contigs with a KEGG ontology annotation. FIG. 8A shows the number of properly paired reads that exist after sequencing and filtering. FIG. 8B indicates the number of contigs assembled by Trinity and those with an annotation.

FIG. 9 shows a heatmap of TMM-normalized CPM expression values of the top 10,000 differentially expressed genes. Samples and genes are hierarchically clustered based on the Complete linkage method.

FIGS. 10A-10C show MA plots for changes in gene expression between relative humidity ERH conditions of 100% vs 85% as in FIG. 10A, 100% vs 50% as shown in FIG. 10B, and 50% vs 85% as shown in FIG. 10C. There are 61,956 statistically significant DE Trinity genes between 100% and 85% ERH; 54,673 between 100% and 50% ERH; and 47,078 between 50% and 85% ERH. Significantly differentially expressed contigs (p_{adj}<0.05) are in red. MA plots transform the counts onto log 2 ratio (M, y-axis) and average log 2 (A, x-axis) scales.

FIG. 11 shows a Spearman correlation heatmap of differentially expressed genes in all pairwise comparisons having log 2FC≥2 and p≤0.001. Log2 transformed and TMM-normalized Counts Per Million values are used. Rows and columns are ordered based on hierarchical clustering based on the Complete linkage method. Correlation coefficients that are not significant (p>0.05) are shown as blank tiles on the heatmap.

FIG. 12 shows the number of significantly upregulated and downregulated genes in each pairwise comparison for overall genes expressed and fungal annotated genes, where upregulated and downregulated genes had a log 2FC>|2| and FDR-adjusted p value<0.05.

FIG. 13 shows the number of genes present in the dust at different ERH levels after one week.

FIG. 14 shows a metabolic pathway map of fungal genes upregulated at 50% ERH when compared to 100% ERH.

FIGS. 15A-15C shows images showing the increase in the number of upregulated fungal metabolic pathways at 50% ERH (50% vs 100%) in FIG. 15A, to 85% (85% vs 50%) in FIG. 15B and to 100% ERH (50% vs 100%) in FIG. 15C.

FIG. 16 shows fungal concentrations (spore equivalents per mg dust) in the dust at different ERH levels after one week.

FIG. 17 shows composition of fungal taxa at 50% ERH after one week.

FIG. 18 shows a heatmap of TMM normalized CPM expression values of target genes upregulated at 85% ERH (compared to 50%). Darker color intensities represent higher gene expression values. The bottom of the heatmap shows state locations ordered from west to east. Genes are ordered alphabetically.

### DETAILED DESCRIPTION

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

The term “antibody” is used in the broadest sense, and specifically covers monoclonal antibodies (including full length monoclonal antibodies), polyclonal antibodies, and multispecific antibodies (e.g., bispecific antibodies). Native antibodies and immunoglobulins are usually heterotetrametric glycoproteins of about 150,000 Daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end. Antibodies (Abs) exhibit binding specificity to a specific target. Antibody specificity can be assessed by comparing binding signals in cells expressing the target protein to control cells with the target gene knocked out. A highly specific antibody should show no binding activity if the target is not there. With protein antigens, the antibody molecule contacts the antigen over a broad area of its surface that is complementary to the surface recognized on the antigen. Electrostatic interactions, hydrogen bonds, van der Waals forces, and hydrophobic interactions can all contribute to binding.

The term “antimicrobial” refers to an agent that kills microorganisms or stops their growth.

The term “antibacterial” refers to an agent that is proven to kill bacteria or stops bacterial growth.

The term “antibiotics” refers to a type of antimicrobial substance active against bacteria. These are the most important type of antimicrobial agent for fighting bacterial infections, and antibiotics medications are widely used in the treatment and prevention of such infections. They may either kill or inhibit the growth of bacteria.

“Built environment” as used herein is any human-made, naturally-occurring or modified structure, including commercial, retail, private, governmental, educational, temporary, vehicular, and recreational structures.

As used herein, the term “buffer” refers to a solution consisting of a mixture of acid and its conjugate base, or vice versa. The solution is used as a means of keeping the pH at a nearly constant range to be used in a wide variety of chemical and biological applications.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Culture” or “cell culture” is the process by which cells are grown under controlled conditions, generally outside their natural environment. After the cells of interest have been isolated from living tissue, they can subsequently be maintained under carefully controlled conditions. These conditions vary for each cell type, but generally consist of a suitable vessel with a substrate or medium that supplies the essential nutrients (amino acids, carbohydrates, vitamins, minerals), growth factors, hormones, and gases (CO₂, O₂), and regulates the physio-chemical environment (pH buffer, osmotic pressure, temperature). Most cells require a surface or an artificial substrate to form an adherent culture as a monolayer (one single-cell thick), whereas others can be grown free floating in a medium as a suspension culture. “Cell culture” also refers to the culturing of cells derived from multicellular eukaryotes, especially animal cells, in contrast with other types of culture that also grow cells, such as plant tissue culture, fungal culture, and microbiological culture (of microbes).

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “detect” or “detecting” refers to an output signal released for the purpose of sensing of physical phenomenon. An event or change in environment is sensed and signal output released in the form of light.

An expression “database” denotes a set of stored data that represents a collection of sequences, which in turn represent a collection of biological reference materials.

“Differentially expressed” as applied to a gene, refers to the differential production of the mRNA transcribed from the gene, or the protein product encoded by the gene. A differentially expressed gene may be overexpressed or under expressed as compared to the expression level of a normal or control cell. In one aspect, it refers to a differential that is 2.5 times, preferably 5 times, or preferably 10 times higher or lower than the expression level detected in a control sample. The term “differentially expressed” also refers to nucleotide sequences in a cell or tissue which are expressed where silent in a control cell or not expressed where expressed in a control cell.

As specified herein, the term “DNA construct” refers to a sequence of deoxyribonucleotides including deoxyribonucleotides obtained from one or more sources.

“Expression” as used herein refers to the process by which information from a gene is used in the synthesis of a functional gene product that enables it to produce a peptide/protein end product, and ultimately affect a phenotype, as the final effect.

The term “gene” as used in this specification refers to a segment of deoxyribonucleotides (DNA) possessing the information required for synthesis of a functional biological product such as a protein or ribonucleic acid (RNA).

The term “genetic engineering” is used to indicate various methods involved in gene manipulation including isolation, joining, introducing of gene(s) as well as methods to isolate select organisms containing the manipulated gene(s).

The term “gene expression” refers to efficient transcription and translation of genetic information contained in concerned genes.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “kit” describes a wide variety of bags, containers, carrying cases, and other portable enclosures which may be used to carry and store solid substances, liquid substances, and other accessories necessary to detect microbial growth in a built environment. Such kits and their contents along with any applicable procedures may be used to provide access to better health outcomes in accordance with the teachings of the present disclosure.

As used herein, the term “lysis” refers to the process of breaking down the membrane of a cell, often by viral, enzymatic, or osmotic mechanisms that compromise cellular integrity.

The terms “metabolite” or “metabolic compound” as used herein refers to small molecules that are generally intermediates or end products of a metabolic pathway or process.

A “mitochondrion” is a cellular membrane-bound compartment, or organelle found in most eukaryotic cells, which are essential for cellular respiration and cellular energy production. These cellular structures comprise their own genome consisting of 37 genes important for energy production, respiration, calcium regulation, heat generation, and mediating cell growth and death.

The term “microorganism” mentioned herein refers to one or more forms/species of bacteria or fungi.

The term “nucleic acid” as used herein means natural and synthetic DNA, RNA, oligonucleotides, oligonucleosides, and derivatives thereof. For ease of discussion, such nucleic acids are at times collectively referred to herein as “constructs,” “plasmids,” or “vectors.”

As used herein, the term “polymerase” refers to an enzyme that synthesizes long chains of polymers or nucleic acids. DNA polymerase and RNA polymerase are used to assemble DNA and RNA molecules, respectively, by copying a DNA template strand using base-pairing interactions.

As used herein, the term “polymerase chain reaction” (“PCR”) refers to a method for increasing the concentration of a segment of a target sequence in a mixture of genomic DNA without cloning or purification. This process for amplifying the target sequence typically consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured, and the primers then annealed to their complementary sequences within the target molecule. Following annealing, the primers are extended with a polymerase so as to form a new pair of complementary strands. The steps of denaturation, primer annealing, and polymerase extension can be repeated many times to obtain a high concentration of an amplified segment of the desired target sequence. Unless otherwise noted, PCR, as used herein, also includes variants of PCR such as allele-specific PCR, asymmetric PCR, hot-start PCR, ligation-mediated PCR, multi-plex-PCR, reverse transcription PCR, or any of the other PCR variants known to those skilled in the art.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

The term “recombinant” cells or population of cells refers to cells or population of cells into which an exogenous nucleic acid sequence is introduced using a delivery vehicle such as a plasmid.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Disclosed are the components to be used to prepare the disclosed kits as well as to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular kit is disclosed and discussed and a number of modifications that can be made to the kit components are discussed, specifically contemplated is each and every combination and permutation of the kit components and the modifications that are possible unless specifically indicated to the contrary. Thus, if a set of kit components A, B, and C are disclosed as well as a set of kit components D, E, and F and an example of a combination of the components, or, for example, a combination of kit components comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the methods and kits disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

### DESCRIPTION OF THE INVENTION

Mold species can vary in damp areas, and secondary metabolic processes in mold can be independent of species. This invention provides an evidence-based measurement target for evaluation of mold growth in built environments based on species-independent metabolic processes. In short, products from secondary metabolic pathways of fungi are species-independent and are more effective indicators of mold growth than measurement of any specific species. The methods and kits herein are based on identification of nucleic acids, proteins, metabolites, volatile organic compounds, chemicals or a combination thereof that are differentially expressed when microbes are growing in a built environment. These nucleic acids and/or proteins can serve as targets in a quantitative microbial growth measurement method. The targets can be detected in a variety of ways discussed herein.

Disclosed herein is a metatranscriptomic pipeline to analyze gene expression of microbial communities in house dust. To do this, first the challenges associated with high levels of RNases in dust and bioinformatic processing were overcome. Interestingly, these patterns in gene expression have important implications. For instance, many allergens are present in secondary metabolic pathways. This results in increased gene expression of allergenic proteins at higher moisture levels.

Fortunately, many secondary metabolic pathways in filamentous fungi have been elucidated because of interest in drug development. Many of these processes are consistent throughout the fungal kingdom, such as those related to germination and growth, and thus are independent of the presence of any given species.

The invention provides a quantitative measurement technique that avoids subjectivity in microbial growth assessment and more robust results, which was a long-felt need. The lack of such a test is partially due to the complex nature of these indoor exposures. Each home contains a unique and diverse microbial community that varies based on surface type, as well as a complex mixture of chemicals. Microbial species in a home can number in the hundreds to thousands. The present invention provides methods related to indicators inherently associated with the presence of excess moisture and microbial growth. This invention takes advantage of the advent of high-throughput DNA/RNA sequencing, which presents an important opportunity to vastly improve exposure assessment. Previously, the use of culture-based methods to study microbial communities could only reveal a small fraction of these organisms present. For instance, only 17% of fungal species are culturable, and still others might not be detected such as viable-but-not-culturable spores, non-propagating fungal fragments, and species that grow slowly. In contrast, high-throughput (or next-generation) DNA sequencing can identify all species present without a priori selection and can indicate quantitative values when coupled with quantitative polymerase chain reaction (qPCR). Additionally, the use of RNA sequencing reveals microbial function within an entire community. The use of this cutting-edge technology on environmental samples represents an underutilized opportunity to reveal answers to fundamental questions about the microbial processes that occur in damp buildings.

### Methods

Disclosed herein is a method of inhibiting or reducing microbial growth in a built environment, wherein a built environment is a natural or man-made structure, or building wherein people live or work for example a house, laboratory, hospital, manufacturing plant, airport, airplane, school, and office. Increase in the humidity and decrease in ventilation of such a built environment can support the growth of microbes such as bacteria and fungi, especially mold.

Mold is a type of fungi and can be broadly classified into three types: Allergenic, Pathogenic and Toxigenic. Allergenic mold species are those that trigger allergic reactions such as asthma. Some examples for allergenic mold species are Chaetomium, Alternaria, Ulocladium, Serpula, Mucor, Aureobasidium and Penicillium. Pathogenic mold species cause disease in immunocompromised individuals. In some embodiments, the pathogenic mold species is Aspergillus. Toxigenic mold species create and produce their own toxins which can lead to health problems that are sometimes lethal. In some embodiments, the toxigenic mold species are Stachybotrys or black mold and Trichoderma. It is most common for mold to grow in houses damaged by flooding and large water leaks, and with poor air quality. Stachybotrys is associated with sick building syndrome. This mold can be dangerous and needs to be removed only by a licensed remediation specialist who can treat the built environment affected by mold by eliminating excess moisture.

In some examples, the built environment can have an equilibrium relative humidity (ERH) of 30%-100%. As further disclosed herein ERH is the relative humidity of the atmosphere at a particular temperature at which a material neither gains nor loses moisture. In some embodiment of the disclosure herein, the ERH can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any amount below or in-between these values. Further disclosed herein is the first step in the method of inhibiting or reducing microbial growth is identifying the microbial growth by detecting one or more gene(s) or product(s) thereof associated with the fungal growth processes including but not limited to sporulation, hyphal growth and conidium formation and other fungal growth-related functional processes in at least one sample collected from the built environment. In some embodiments the sample can be a dust sample, a surface sample, an air sample, and/or a combination of environmental samples. Sporulation is the process by which a vegetative cell undergoes a developmental change to form a metabolically inactive spore, or endospore in the scarcity of nutrition and optimal growth conditions.

In some embodiments, fungal growth and sporulation genes detected or products thereof are selected from Table 2. In some embodiments, the genes include, ALTA7, atfB, catA, hsp30, nuo-49, rodA, wA, arp1, arp2, gel2, gna-3, mpg1, mtlD, pkaR, tpsA, velB, vosA, wetA, Canx homolog, tpiA, camA, crzA, ecm33, hex1, bimG, mdm10, cap2, catB, chsA, gpaA, nudA, rhoC, steA, candA-C, candA-N, ccg-8, dop1, flbA, fluG, laeA, nudG, sidH, chsC, grrA, hymA, or sidC or a combination thereof. In some embodiments the genes or products thereof can be fungal growth-related morphological changes, stress response, mitochondria and secondary metabolism, and other metabolic processes. In one example, quantitative polymerase chain reaction (PCR) can be used to detect and quantify the expression of the genes listed. Lateral flow chromatography is used to quantify the product(s) thereof. The increase in the gene(s) expression or the quantity of the product(s) in collected samples as compared to the levels in controls can indicate microbial growth. In some embodiments, the quantity of the microbes identified are compared to a control with a threshold value, database value, normalized value, relative value, validated value, or a combination thereof. This control can be from a different environment, or the same environment at different time point (or multiple previous time points).

Once the gene expression is identified to indicate microbial growth a treatment can be applied to inhibit the microbial growth. In some embodiments, the microbial growth inhibition techniques comprise the use of a dehumidifier, an exhaust fan, an anti-microbial compound, a hydrophobic paint, or a combination thereof. In some embodiments, the treatment can be administered hourly, every 2, 3, 4, 5, 6, 7, 8, 9, 10, 11, 12, 13, 14, 15, 16, 17, 18, 19, 20, 21, 22, 23 hours, daily once, twice or three times weekly, monthly for up to 1, 2, 3 week(s), 1, 2, 3, 4, 5, 6, 7, 8, 9, 10, 11 month(s), 1, 2, or 3 years. In some embodiments the anti-microbial treatment can be anti-fungal and/or antibacterial.

In some embodiments, an anti-fungal agent is selected from the group comprising (3-ethoxypropyl) mercury bromide, 2-methoxyethylmercury chloride, 2-phenylphenol, 2,4,5-trichlorophenol, 2,2-dibromo-3-nitrilopropionamide, 8-hydroxyquinoline, 8-phenylmercurioxyquinoline, acibenzolar, acypetacs, albendazole, aldimorph, allicin, allyl alcohol, allyl isothiocyanate, ametoctradin, aminopyrifen, amisulbrom, amobam, ampropylfos, anilazine, asomate, aureofungin, azaconazole, azithiram, azoxystrobin, barium polysulfide, benalaxyl, benodanil, benomyl, benquinox, bentaluron, benthiavalicarb, benzalkonium chloride, benzamacril, benzamide fungicides, benzamorf, benzimidazole fungicides, benzohydroxamic acid, benzovindiflupyr, berberine, bethoxazin, bifujunzhi, binapacryl, biphenyl, bitertanol, bithionol, bixafen, blasticidin-S, Bordeaux mixture, boric acid, boscalid, bromothalonil, bromuconazole, bronopol, bupirimate, Burgundy mixture, buthiobate, sec-butylamine, calcium polysulfide, captafol, captan, carbamorph, carbendazim, carboxin, carpropamid, carvacrol, carvone, cetoctaelat, Cheshunt mixture, chinomethionat, chitosan, chlobenthiazone, chloraniformethan, chloranil, chlorfenazole, chlorodinitronaphthalene, chloroinconazide, chloroneb, chloropicrin, chlorothalonil, chlorquinox, chlozolinate, ciclopirox, climbazole, clotrimazole, copper (II) acetate, copper (II) carbonate, copper hydroxide, copper naphthenate, copper oleate, copper (I) oxide, copper oxychloride, copper (II) sulfate, copper zinc chromate, coumoxystrobin, cresol, cufraneb, cuprobam, cyanogen, cyazofamid, cyclafuramid, cyclobutrifluram, cycloheximide, cyflufenamid, cymoxanil, cypendazole, cyproconazole, cyprodinil, cyprofuram, dazomet, DBCP, debacarb, decafentin, dehydroacetic acid, dicarboximide fungicides, dichlobentiazox, dichlofluanid, dichlone, dichlorophen, dichlozoline, diclobutrazol, diclocymet, diclomezine, dicloran, diethofencarb, diethyl pyrocarbonate, difenoconazole, diflumetorim, dimefluazole, dimetachlone, dimethirimol, dimethomorph, dimethyl disulfide, dimoxystrobin, diniconazole, dinobuton, dinocap, dinocton, dinopenton, dinosulfon, dinoterbon, diphenylamine, dipymetitrone, dipyrithione, disodium octaborate, disulfiram, ditalimfos, dithianon, DNOC, dodemorph, dodicin, dodine, drazoxolon, edifenphos, enoxastrobin, epoxiconazole, etaconazole, etem, ethaboxam, ethirimol, ethoxyquin, ethylene oxide, ethylicin, ethylmercury 2,3-dihydroxypropyl mercaptide, ethylmercury acetate, ethylmercury bromide, ethylmercury chloride, ethylmercury phosphate, etridiazole, famoxadone, fenamidone, fenaminosulf, fenaminstrobin, fenapanil, fenarimol, fenazaquin, fenbuconazole, fenfuram, fenhexamid, fenitropan, fenjuntong, fenoxanil, fenpiclonil, fenpicoxamid, fenpropidin, fenpropimorph, fenpyrazamine, fentin, ferbam, ferimzone, florylpicoxamid, fluazinam, flubeneteram, fluconazole, fludioxonil, flufenoxadiazam, flufenoxystrobin, fluindapyr, flumetover, flumorph, fluopicolide, fluopimomide, fluopyram, fluoroimide, fluotrimazole, fluoxapiprolin, fluoxastrobin, fluoxytioconazole, fluquinconazole, flusilazole, flusulfamide, flutianil, flutolanil, flutriafol, fluxapyroxad, folpet, formaldehyde, fosetyl, fuberidazole, furalaxyl, furametpyr, furcarbanil, furconazole, furfural, furmecyclox, furophanate, glyodin, griseofulvin, guazatine, halacrinate, hexachlorobenzene, hexachlorobutadiene, hexachlorophene, hexaconazole, hexylthiofos, hanjunzuo, hydrargaphen, hymexazol, imazalil, imibenconazole, iminoctadine, inezin, inpyrfluxam, iodocarb, ipconazole, ipfentrifluconazole, ipflufenoquin, iprobenfos, iprodione, iprovalicarb, isavuconazole, isofetamid, isoflucypram, isoprothiolane, isopyrazam, isotianil, isovaledione, itraconazole, izopamfos, jiaxiangjunzhi, kasugamycin, kejunlin, ketoconazole, kresoxim-methyl, Lime sulfur, manam, mancopper, mancozeb, mandestrobin, mandipropamid, maneb, mebenil, mecarbinzid, mefentrifluconazole, mepanipyrim, mepitriflufenpyr, mepronil, meptyldinocap, mercuric chloride, mercuric oxide, mercurous chloride, metalaxyl, metam, metarylpicoxamid, metazoxolon, metconazole, methasulfocarb, methfuroxam, methyl bromide, methyl isothiocyanate, methylmercury benzoate, methylmercury dicyandiamide, methylmercury pentachlorophenoxide, metiram, metomeclan, metominostrobin, metrafenone, metsulfovax, metyltetraprole, milneb, moroxydine, myclobutanil, myclozolin, N-(ethylmercury)-p-toluenesulfonanilide, nabam, natamycin, ningnanmycin, nystatin, β-nitrostyrene, nitrothal-isopropyl, nuarimol, OCH, octhilinone, ofurace, organotin fungicides (obsolete), orthophenyl phenol, orysastrobin, osthol, oxadixyl, oxine copper, oxpoconazole, oxycarboxin, oxyfenthiin, paclobutrazol, parinol, pefurazoate, penconazole, pencycuron, penflufen, pentachlorophenol, penthiopyrad, phenamacril, phenylmercuriurea, phenylmercury acetate, phenylmercury chloride, phenylmercury derivative of pyrocatechol, phenylmercury nitrate, phenylmercury salicylate, phosdiphen, phthalide, picarbutrazox, picoxystrobin, piperalin, polycarbamate, polyoxins, polyoxorim, posaconazole, potassium azide, potassium polysulfide, potassium thiocyanate, pramiconazole, probenazole, prochloraz, procymidone, propamocarb, propiconazole, propineb, proquinazid, prothiocarb, prothioconazole, pydiflumetofen, pyracarbolid, pyraclostrobin, pyrametostrobin, pyraoxystrobin, pyrapropoyne, pyraziflumid, pyrazole fungicides, pyrazophos, pyribencarb, pyributicarb, pyridachlometyl, pyridinitril, pyrifenox, pyrimethanil, pyriofenone, pyrisoxazole, pyroquilon, pyroxychlor, pyroxyfur, quinacetol, quinazamid, quinconazole, quinofumelin, quinoxyfen, quintozene, rabenzazole, ravuconazole, saijunmao, saisentong, salicylanilide, sanguinarine, santonin, seboctylamine, sedaxane, silthiofam, silver, simeconazole, sodium azide, sodium bicarbonate, sodium orthophenylphenoxide, sodium pentachlorophenoxide, sodium polysulfide, spiroxamine, streptomycin, strobilurin fungicides, sulfur, sulfuryl fluoride, sultropen, TCMTB, tebuconazole, tebufloquin, tecloftalam, tecnazene, tecoram, tetraconazole, thiabendazole, thiadifluor, thicyofen, thifluzamide, thiochlorfenphim, thiocyanatodinitrobenzenes, thiodiazole-copper, thiomersal, thiophanate, thiophanate-methyl, thioquinox, thiram, thujaplicins, thymol, tiadinil, tioxymid, tolclofos-methyl, tolfenpyrad, tolnaftate, tolnifanide, tolprocarb, tolylfluanid, tolylmercury acetate, triadimefon, triadimenol, triamiphos, triarimol, triazbutil, triazoxide, tributyltin oxide, trichlamide, trichlorotrinitrobenzenes, triclopyricarb, tricyclazole, tridemorph, trifloxystrobin, triflumizole, triforine, trimorphamide, triticonazole, Undecylenic acid, uniconazole, urbacide, validamycin, valifenalate, vangard, vinclozolin, voriconazole, zarilamid, zinc naphthenate, zineb, ziram or a combination thereof.

In some embodiments, the antibacterial is an antibiotic. In some embodiments, the antibiotic is selected from a group including, but not limited to penicillins (including, but not limited to amoxicillin, clavulanate and amoxicillin, ampicillin, dicloxacillin, oxacillin, and penicillin V potassium), tetracyclins (including, but not limited to demeclocycline, doxycycline, eravacycline, minocycline, omadacycline, sarecycline, and tetracycline), cephalosporins (cefaclor, cefadroxil, cefdinir, cephalexin, cefprozil, cefepime, cefiderocol, cefotaxime, cefotetan, ceftaroline, cefazidme, ceftriaxone, and cefuroxime), quinolones (also referred to as fluoroquinolones include, but are not limited to ciprofloxacin, delafloxacin, levofloxacin, moxifloxacin, and gemifloxacin), lincomycins (including clindamycin and lincomycin), macrolides (including, but not limited to azithromycin, clarithromycin, erythromycin, and fidaxomicin (ketolide)), sulfonamides (including sulfamethoxazole and trimethoprim, and sulfasalazine), glycopeptides (including, but not limited to dalbavancin, oritavancin, telavancin, and vancomycin), aminoglycosides (including, but not limited to gentamicin, tobramycin, and amikacin), carbapenems (including, but not limited to imipenem and cilastatin, meropenem, and ertapenem), and topical antibiotics (including, but not limited to neomycin, bacitracin, polymyxin B, and praxomine) used alone or in combination.

Also disclosed herein is a method of detecting and identifying microbial growth by identifying one or more gene(s) or product(s) thereof, wherein the microbe is a bacterium, fungi, or protozoan. Further, a product is a compound produced by a cell metabolism and excreted to the extracellular medium. As disclosed herein the extracellular medium can be air or soil inside the built environment. Some examples of microbial gene(s) or product(s) thereof are ALTA7, atfB, catA, hsp30, nuo-49, rodA, wA, arp1, arp2, gel2, gna-3, mpg1, mtlD, pkaR, tpsA, velB, vosA, wetA, Canx homolog, tpiA, camA, crzA, ecm33, hex1, bimG, mdm10, cap2, catB, chsA, gpaA, nudA, rhoC, steA, candA-C, candA-N, ccg-8, dop1, flbA, fluG, laeA, nudG, sidH, chsC, grrA, hymA, or sidC. These products can be detected by nucleic acid or protein-based detection assays. In some embodiments, the detection assays can be quantitative polymerase chain reaction (qPCR), lateral flow chromatography, colorimetric dye, fluorescent dye, Biuret, Bradford, bicinchoninic, Folin-Lowry, Kjeldahl, antibody binding, ultraviolet light absorbance, gel electrophoresis, capillary electrophoresis, diphenylamine, polymerase chain reaction, RFLP analysis, and/or a combination thereof. As shown herein, the one or more gene(s) or product(s) thereof are measured by identifying protein, RNA, and/or DNA in the sample. As shown here, the one or more gene(s) or product(s) thereof is identified by detecting and/or quantifying the expression of one or more gene(s). In some embodiments, the one or more gene(s) are either genomic and/or mitochondrial and are measured using quantitative polymerase chain reaction (qPCR).

In some embodiments, the ALTA7, atfB, catA, hsp30, nuo-49, rodA, wA, arp1, arp2, gel2, gna-3, mpg1, mtlD, pkaR, tpsA, velB, vosA, wetA, Canx homolog, tpiA, camA, crzA, ecm33, hex1, bimG, mdm10, cap2, catB, chsA, gpaA, nudA, rhoC, steA, candA-C, candA-N, ccg-8, dop1, flbA, fluG, laeA, nudG, sidH, chsC, grrA, hymA, or sidC are associated with a fungal growth process such as hyphal extension, sporulation and/or a combination thereof.

In some embodiments, the ALTA7, atfB, catA, hsp30, nuo-49, rodA, wA, arp1, arp2, gel2, gna-3, mpg1, mtlD, pkaR, tpsA, velB, vosA, wetA, Canx homolog, tpiA, camA, crzA, ecm33, hex1, bimG, mdm10, cap2, catB, chsA, gpaA, nudA, rhoC, steA, candA-C, candA-N, ccg-8, dop1, flbA, fluG, laeA, nudG, sidH, chsC, grrA, hymA, or sidC are found in multiple taxa in the fungal kingdom, including Aspergillus nidulans, Neurospora crassa, Myxococcus xanthus, Saccharomyces Cerevisiae and other fungal taxa.

### Kits

Disclosed herein is a kit for the detection of microbial growth in a built environment, wherein the kit is used to identify and quantify expression of one or more gene(s) or product(s) thereof in one or more microbe(s) in at least one sample obtained from a built environment. As disclosed herein, the kit comprises a sample collection device. In some embodiments, the sample collection device is selected from a group comprising of a swab, a brush, sterile tubes with lids, vacuum cleaner with a sterile collection bag, a canister, a zip-top bag, or a combination thereof for the sterile collection of samples, wherein a sample is a dust sample, a surface sample, an air sample, and/or a combination of environmental samples.

Also disclosed herein, the kit further can comprise a glass chamber, for incubating the soil samples collected from the built environment and a salt solution or distilled water to maintain relative humidity along with a AquaLab™ dew point water activity meter to measure the relative humidity of the sample. As disclosed herein, the kit can further comprise a sample resuspension buffer, a lysis buffer, a wash buffer, a phenol, and chloroform for extraction of nucleic acids and proteins. Wherein, during the phenol-chloroform extraction, a mixture of phenol, chloroform, and isoamyl alcohol is added to samples to promote the partitioning of proteins, lipids and debris into an organic phase, leaving the DNA in the aqueous phase. Further enclosed in the kit are one or more control sample(s), a nucleic acid or protein detection probe, DNA or RNA polymerase and thermocycler or a lateral flow chromatography device. In some embodiments, the nucleic acid detection probe is a pair of forward and reverse primers and the expression of the one or more gene(s) is identified and quantified by quantitative polymerase chain reaction (qPCR).

In some embodiments, the protein detection probe can be an antibody, and the one or more product(s) thereof is detected in a whole protein lysate obtained from the at least one sample by lateral flow chromatography wherein the lateral flow chromatography device comprises of a protein lysate loading well, protein detection probe bound to a nitrocellulose membrane and a sample running buffer. In some embodiments, decanted sample resuspension buffer can be collected after resuspending the sample and loaded on the lateral flow chromatography device. As disclosed herein the qPCR gene expression and protein density results can be read and quantified via a smart phone-based application. Furthermore, the kit comprises components for comparing the expression of the one or more gene(s) to a control with a threshold value, database value, normalized value, relative value, validated value, or a combination thereof. The increase in the gene(s) expression or the quantity of the product(s) in collected samples as compared to the levels in controls indicates microbial growth.

### EXAMPLES

### Example 1

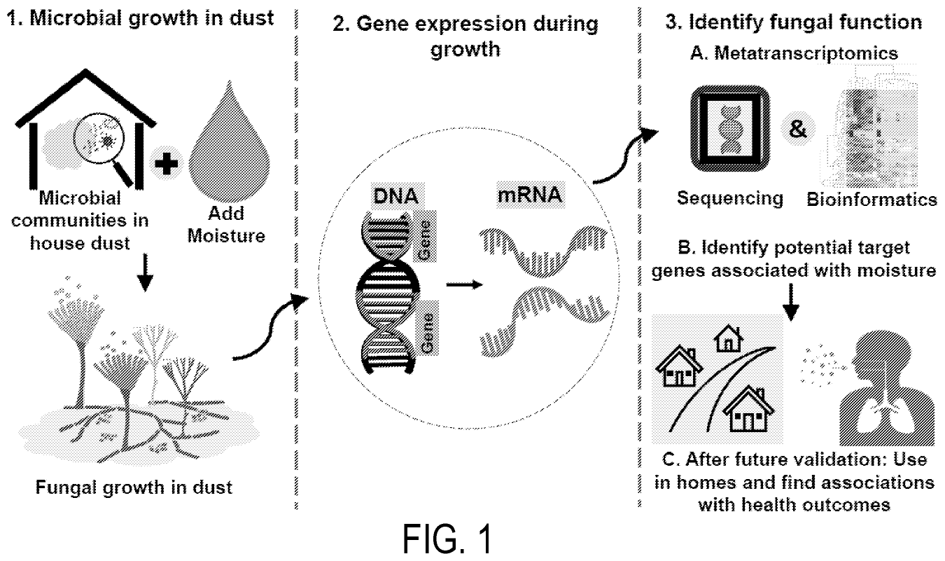

Dust was collected and incubated in laboratory chambers to simulate elevated moisture conditions. First, RNA from 9 sites were screened for increased secondary metabolic pathways associated with elevated moisture. 10 potential target products associated with moisture were identified and then validated through qPCR in 50 sites (FIG. 1).

Recruitment: House dust collection, respiratory health surveys, and environmental surveys were conducted in 50 non-moisture damaged homes (see Power Analysis below). A total of 44 homes were recruited in Columbus, Ohio. Homes were recruited through both community flyers in locations such as libraries and Starbucks, and through social media, which has worked well in the past. In addition, recruitment can be done through environmental practitioner partners of homes undergoing environmental testing for a non-moisture concern, such as radon or asbestos. 6 additional homes were recruited from geographically distinct locations within the US through colleagues in areas such as California to add geographic diversity. Geographic diversity is not expected to change the microbial response to growth, but these samples were included at the beginning to ensure wide applicability. Institutional Review Board (IRB) approval was acquired prior to recruitment.

Dust collection: Floor dust was focused on because 1) it is less variable than air samples and 2) it represents a long-term exposure that could be expected to be stable for about a season. The staff collected house dust samples by vacuuming into a filter using established protocols. A sample was collected from both the main living area and bedroom. Collection from carpets was prioritized to maximize dust collection but was collected from solid surfaces when needed. The goal was to collect >25 g of dust. If insufficient dust (<10 g) was collected (as noted by visual inspection), it was also be collected from upholstered furniture. The occupant was asked for their vacuum bag or for dust in their bagless vacuum.

In-home measurements: In addition to dust collection, information on other environmental variables was also collected. The relative humidity at the center of the room may not accurately reflect the value near the floor, a wall, or a window. Therefore, moisture was measured as well as temperature in all of these locations. The temperature and relative humidity were measured outdoors as well as in the center of both the main living area and the main bathroom. The equilibrium relative humidity was measured on all the walls at the center point on the wall 1.5 m above the floor in a way that is non-destructive. Any areas that have visible water damage or mold growth were also measured, and in Objective 1 these homes were excluded when confirmed.

The EPA's Asthma Home Environment Checklist will be offered to the occupant. A survey was conducted to gain more information about dwelling (age, rental status, condition), pests (cockroaches, mice, rats), pets (dogs, cats, other furry animals, birds, other), number of occupants, heating and cooling systems, whether windows are opened on a regular basis, tobacco product use, cooking habits, and other factors. Other information, such as location, was observed from a Geographic Information System (GIS).

Chamber Experiments: Methods were consistent with previous protocols. Briefly, dust was sieved to 300 am, mixed, 100 mg placed on baking aluminum foil trays, and incubated at 25° C. with set relative humidity levels. Dust was stored at room temperature for the short period of time prior to use to preserve microbial communities. Relative humidity was controlled in each 3.8 L glass chamber using 100 mL of salt solution (NaCl above water activity of 0.76 and MgCl2 below) and verified with an Aqualab 4TE water activity meter (Decagon Devices, Pullman, WA). Relative humidity included the following conditions held for 1 week: 30%, 40%, 50%, 60%, 70%, 80%, 85%, 90%, and 100% RH. Relative humidity was monitored in the chambers using HOBO data loggers (OnSet, Cambridge, MA). Before and after incubation, water activity of the dust was measured in the AquaLab 4TE water activity meter and water content were calculated by changes in dust weight.

Samples analyzed are detailed in Table 1. All samples were analyzed quantitatively by qPCR for fungi and bacteria, and for taxonomic analysis of fungi. All samples underwent ITS sequencing to identify changes in fungal communities. Only a subset of samples (~20%) were analyzed for 16S bacterial sequencing. This analysis focused on fungi based on prior work.

TABLE 1
Objective 1 Samples.

| | | RNA Seq | Total |
|---|---|---|---|
| Condition | RH | Replicates | Replicates |
| 1 | 50% | 9 sites | 50 sites |
| 2 | 85% | 9 sites | 50 sites |
| 3 | 100% | 9 sites | 50 sites |

### Example 2

### Microbial Function (RNA) Analysis

For this experiment, dust samples were incubated at 50%, 85%, and 100% relative humidity for one week in chambers as described above (Table 1). Gene expression and microbial function were evaluated by examining RNA produced from the microbial communities. This was conducted on the 6 geographically distinct samples and an additional 3 samples collected from a single area. The RNA sequencing is a screening step to identify potential targets. It was limited to 9 sites due to the high cost and processing time associated with this process, and 10 potential targets were evaluated by qPCR in all 50 sites.

RNA Extraction and Sequencing: To measure gene expression, RNA was extracted using a method that was utilized previously. The Qiagen Microbiome RNA extraction kit was utilized with a 10× increase in the concentration of β-mercaptoethanol to further prevent RNA degradation from RNases. Extracted RNA was immediately frozen at −80° C. prior to use and transported on dry ice. RNA was sequenced at the Yale sequencing facility using a protocol that had successfully been used to retrieve RNA from dust in the past. Fungal RNA is more important than bacterial RNA due to the broader range of tolerated relative humidity levels in fungi. Therefore, eukaryotic RNA was selected using the polyA protocol. A total of 9 samples were run on a single Illumina NovaSeq lane (3 total lanes will be used), which has provided sufficient coverage in the past.

### Example 3

### Statistical and Bioinformatic Analysis of Microbial Function

The dataset was analyzed to determine overall gene expression trends at different relative humidity levels. Microbial function was compared under both moisture-limited and moisture-rich conditions, and genes present in secondary metabolic pathways were especially considered.

Gene expression was analyzed using Trinity and a previously developed protocol. Briefly, sequence quality assessment was done in FastQC, and poor-quality reads removed with Trimmomatic in Trinity. Trinity's default settings were used to conduct a de novo metatranscriptomic assembly, and all contigs with length less than 300 base pairs were removed. Contigs were clustered using CD-HIT-EST with 80% similarity, and these clusters composed the full transcriptome. BLASTx was used to map contigs to the SwissProt database and NCBI's RefSeq non-redundant (NR) database. Blast2GO was used to generate gene ontology terms. Contigs for the entire microbial communities were mapped onto the KEGG Ontology pathway with GhostKOALA to identify probable microbial functions at different levels of relative humidity.

Enrichment analysis was performed to determine which gene ontology terms were differentially expressed under different relative humidity conditions using both GAGE and Blast2GO. Log2F change was determined for genes based on gene ontology contig mappings. DESeq2 was used to provide log 2F values to GAGE. GAGE was then used to identify gene ontology terms that were significantly associated with a specific condition using Benjamini-Hochberg adjusted p-values with a significance level of 0.05. Changes in gene regulation between conditions were assessed using Fisher's Exact Test in Blast2GO. The full transcriptome was used as the reference set. Gene ontology terms were used with a false discovery rate less than or equal to 0.05. “Core enzymes” were similarly analyzed using a method with a similar protocol.
A. Different processes considered “secondary metabolic processes” were compared and their upregulation at different relative humidity levels. This confirmed the preliminary data from one home that was indicative of the larger number of homes included in this study. Known secondary metabolic processes in fungi are listed in the literature and also on the KEGG Ontology website. In the gene expression data, potential expression of allergens and potential toxins that could be harmful to health was evaluated. It was determined if these were more highly enriched at increased moisture conditions.
B. The genes most strongly and consistently upregulated under damp conditions as potential targets were identified. From this list, a selection of ten compounds was prioritized for further testing by also considering the following parameters: 1) known association with fungal growth processes in the cell such as epA, hypA, and podB-podD that are involved in spore germination and hyphal extension, 2) ease of measurement, and 3) consistency of presence across taxa in the fungal kingdom. Proteases were also considered, which may be produced during growth and are associated with human health effects. Proteases also already have some measurement techniques available that could potentially be modified for use in house dust. A final selection criterium is that the products must be more strongly associated with moisture conditions than any specific fungal species.

qPCR assays were created for these 10 potential target genes and the presence of these targets was measured in cDNA reverse transcribed from RNA from samples incubated at 50%, 85%, and 100% ERH from all 50 sites to validate the results. The top three targets that were the most strongly associated (Kruskal Wallis) with moisture level were selected for analysis in Objective 2.

Power analysis is not appropriate for this type of data. Power calculation in high-throughput omics data is a challenging question when multiple comparison procedures such as FDR are used. Although there is some ongoing effort in this area (see the ongoing NIH R01 grant 1R01CA190766-01A1), no statistically sound method has been proposed and widely accepted by the statistical community yet. It is expected to see statistical differences based on previous studies, which included a much lower sample number (one site with 3 replicates at 3 conditions).

### Example 4

### Microbial Community Analysis

Microbial measurement: To determine microbial communities, dust was extracted with a modified DNA extraction protocol using the MoBio PowerSoil Powerlyzer kit (MoBio, Carlsbad, CA, USA). Growth was analyzed using qPCR with universal fungal and bacterial primers as well as DNA sequencing of the ITS and 16S regions, as described previously. Bioinformatics analysis was conducted with QIIME, BLAST, and FHiTINGS to process reads and assign taxonomy. Sequencing data was made quantitative by multiplying the relative abundance values by total concentration values determined by qPCR. These techniques allowed for full characterization of the microbial communities, including richness, evenness, total concentration, β diversity measures, and taxonomic identification and quantification. Overall, this analysis allowed determining the amount of microbial growth as well as the species that grow, as done in previous studies.

A note on the selection of FHiTINGS for taxonomic analysis: FHiTINGS was selected as the tool to use for taxonomic identification of fungi. Other available tools require clustering of reads prior to identification. Clustering prior to sequence identification could result in misidentification of reads.

### Example 5

### Statistical Analysis of Microbial Communities (See RNA Details Above)

Both fungal and bacterial communities were compared under “normal” (low moisture) and “potentially harmful” (high moisture) conditions. Organisms present only under high moisture conditions were identified.

Statistical analyses were conducted in SAS (SAS Institute Inc., Cary, NC, USA) with a significance level of 0.05. Statistical methods were used to evaluate differences in microbial growth, diversity (richness, evenness), and microbial taxa based on variable relative humidity exposures. Quantitative analysis of sequencing results was conducted by coupling qPCR (absolute abundance) data with the relative abundance results of DNA sequencing. Analysis was conducted as described previously. Associations between specific taxa and incubation conditions were evaluated using the MULTTEST procedure in SAS to control for multiple comparisons, as described previously.

This analysis yielded important insights into the fundamental processes occurring inside fungal cells in damp buildings. Activation of these secondary metabolic pathways was demonstrated upon exposure to dampness is universal across indoor fungal communities independent of presence of different species. Potential universal indicators of microbial growth were identified in buildings to be tested.

A. Three or more of the indicators were measured in 25 damp and 25 control (non-moisture-damaged) homes in Columbus, OH to determine if the indicator is associated with other dampness measures.

Recruitment: House dust collection, respiratory health surveys, and environmental surveys were conducted in 25 moisture-damaged and 25 non-moisture-damaged homes in Columbus, Ohio. Work was performed in collaboration with environmental practitioner partners to recruit moisture-damaged homes and matching control homes receiving other services such as radon testing that are unrelated to moisture. Institutional Review Board (IRB) approval was acquired prior to recruitment. The same surveys indicated in Objective 1 about home characteristics were conducted, and also extent of the moisture damage.

Development of detection assays: Three assays were developed or utilized to detect microbial products identified in Objective 1 as associated with damp buildings. Inclusion of proteins that have established or existing commercially-available assays were prioritized that can be purchased for ease of use, such as those for proteases. Alternatively, an assay using the proteomics core on campus can be developed. Finally, if that does not work, an RNA extraction was conducted on the dust and use quantitative PCR for detection after creation of cDNA. Previously-developed assays were used from the literature, or assays in-house using NCBI's primer BLAST program was developed.

Sample Analysis: The three selected moisture indicators identified in Objective 1 were measured in all of the homes in at least two locations. In damaged homes, one location was close to the damage (same room), and another was far from the damage (adjacent room). In non-moisture damaged homes, two adjacent rooms were selected for sampling, with preferential selection of the living room.

Alternative plan: Currently, Objective 2 is dependent upon identification of suitable measurement targets. If an appropriate measurement target is not identified, that is also useful information in understanding the complex diversity of metabolic processes that occurs in homes. In this case, dust in homes was collected and stored immediately in RNAlater (a safe RNA preservative produced by Qiagen). RNA sequencing was conducted on the samples to see if the metabolic diversity and secondary metabolic processes overall are upregulated in homes with known moisture problems compared to control homes. For this alternative plan, the number of included homes was reduced to 9 moisture-damaged and 9 non-moisture damaged homes due to cost constraints, but this still exceeds the number expected to be needed as indicated by power calculation.

Statistical Analysis: The goal was to compare the three new indicators of microbial growth with indicators of moisture and dampness in homes. It was determined if each indicator is statistically significantly associated with moisture problems, and the sensitivity and specificity of each indicator was also calculated. This was done with both homes' selection (select as either moisture damaged or non-moisture damaged) as well as with other more specific indicators of damage such 1) visible mold growth 2) visible water damage 3) elevated relative humidity >75% 4) elevated water activity measure on walls >75% 5) moldy odor 6) plumbing leaks and 7) roof/exterior leaks. These indicators were considered both individually and combined together. This information was compared to the 3 most statistically significant fungal species identified in Objective 1. This analysis was repeated separately in samples both collected in the same room as the damage and in the adjacent rooms.

Power analysis: A power analysis was used to determine how many homes to include in this section of the study. The most analogous comparison in the literature was comparison of the presence of known cockroach to the presence of a cockroach indicator in dust (allergen Bla g 1). Data from mold indicators are not as relevant because the indicator is not a specific species. Using the data presented in this study of kitchens, inclusion of 14 homes in the analysis would be sufficient for the study to be powered at 0.80 at a significance level of 0.05. However, this comparison involves substantial uncertainty. Therefore, it has been chosen to increase the number of included homes to 50 to ensure that the associations of interest are evident.

This analysis offers the ability to identify which microbial indicator(s) of dampness are most statistically significantly associated with other indicators of dampness in homes. It was also demonstrated that this indicator of microbial growth is more statistically significantly associated with moisture damage than any specific fungal species. This provides important insights into the processes occurring inside fungal cells, and also identifies an important indicator of dampness that can be integrated into a measurement system in the future. This will be done collaboratively with environmental practitioners to ensure that the indicator is usable in the field.

TABLE 2
Target genes upregulated at 100% or 85% or both, comparing 100% vs 50% or
100% vs 85% or 85% vs 50% and associated with fungal growth processes.

| | | | | Number of sites the |
|---|---|---|---|---|
| | Swissprot | | | gene was expressed |
| Trinity Gene | annotation | Gene | Target gene group | at 50% |
| TRINITY_DN8671_c0_g1 | ALTA7_ALTAL | ALTA7 | Unregulated at 100% ERH | 0 |
| TRINITY_DN60428_c0_g1 | ATFB_ASPPU | atfb | Unregulated at 100% ERH | 0 |
| TRINITY_DN22030_c0_g1 | CATA_ASPFU | catA | Unregulated at 100% ERH | 0 |
| TRINITY_DN8361_c0_g1 | HSP30_EMENI | hsp30 | Unregulated at 100% ERH | 0 |
| TRINITY_DN5894_c0_g2 | NDUS2_NEUCR | nuo-49 | Unregulated at 100% ERH | 0 |
| TRINITY_DN1656_c0_g1 | RODL_EMENI | rodA | Unregulated at 100% ERH | 0 |
| TRINITY_DN372_c0_g1 | WA_EMENI | WA | Unregulated at 100% ERH | 0 |
| TRINITY_DN13900_c0_2g | ARP1_ASPFU | arp1 | Unregulated at 100% ERH | 0 |
| TRINITY_DN13993_c0_g1 | ARP2_ASPFU | arp2 | Unregulated at 100% ERH | 0 |
| TRINITY_DN8726_c0_g1 | GEL2_ASPFU | gel2 | Unregulated at 100% ERH | 0 |
| TRINITY_DN34293_c0_g1 | GPA3_NEUCR | gna-3 | Unregulated at 100% ERH | 0 |
| TRINITY_DN46210_c0_g1 | MPG1_EMENI | mpg1 | Unregulated at 100% ERH | 0 |
| TRINITY_DN18611_c0_g1 | MTLD_PENRW | mtlD | Unregulated at 100% ERH | 0 |
| TRINITY_DN8019_c0_g1 | KAPR_ASPFU | pkaR | Unregulated at 100% ERH | 0 |
| TRINITY_DN2023_c0_g1 | TPS1A_ASPFU | tpsA | Unregulated at 100% ERH | 0 |
| TRINITY_DN1053_c0_g4 | VELB_PENRW | velB | Unregulated at 100% ERH | 0 |
| TRINITY_DN2690_c0_g1 | VOSA_PENRW | vosA | Unregulated at 100% ERH | 0 |
| TRINITY_DN285_c0_g2 | WETA_PEND2 | wetA | Unregulated at 100% ERH | 0 |
| TRINITY_DN3379_c0_g1 | ALP2_ASPFU | alp2 | Unregulated at 100% ERH | 0 |
| TRINITY_DN6303_c0_g1 | AYG1_ASPFU | ayg1 | Unregulated at 100% ERH | 0 |
| TRINITY_DN19513_c0_g2 | CCG6_NEUCR | ccg-6 | Unregulated at 100% ERH | 0 |
| TRINITY_DN1945_c0_g1 | GEL1_ASPFU | gel1 | Unregulated at 100% ERH | 0 |
| TRINITY_DN166_c0_g4 | KATG_EMENI | katG | Unregulated at 100% ERH | 0 |
| TRINITY_DN5789_c0_g1 | NRC2_NEUCR | nrc-2 | Unregulated at 100% ERH | 0 |
| TRINITY_DN51057_c0_g1 | ABR1_ASPFU | abr1 | Unregulated at 100% ERH | 0 |
| TRINITY_DN13539_c0_g1 | AP1_EMENI | napA | Unregulated at 100% ERH | 0 |
| TRINITY_DN17345_c0_g2 | SUN1_ASPFU | sun1 | Unregulated at 100% ERH | 0 |
| TRINITY_DN35090_c0_g1 | ORYZ_EMENI | alp1 | Unregulated at 100% ERH | 0 |
| TRINITY_DN81095_c0_g1 | BRLA_PENCA | brlA | Unregulated at 100% ERH | 0 |
| TRINITY_DN3575_c0_g1 | CALX ASPFU | Canx homolog | Unregulated at 85% and 100% ERH | 0 |
| TRINITY_DN11730_c0_g1 | TPIS_EMENI | tpiA | Unregulated at 85% and 100% ERH | 0 |
| TRINITY_DN12451_c0_g1 | CALM_EMENI | camA | Unregulated at 85% and 100% ERH | 0 |
| TRINITY_DN10372_c0_g1 | CRZA ASPFU | crzA | Unregulated at 85% and 100% ERH | 0 |
| TRINITY_DN20577_c0_g2 | ECM33_ASPFU | ecm33 | Unregulated at 85% and 100% ERH | 0 |
| TRINITY_DN8240_c0_g2 | HEX1 EMENI | hex1 | Unregulated at 85% and 100% ERH | 0 |
| TRINITY_DN7974_c0_g2 | PP1_EMENI | bimG | Unregulated at 85% and 100% ERH | 0 |
| TRINITY_DN5762_c0_g1 | MDM10_EMENI | mdm10 | Unregulated at 85% and 100% ERH | 0 |
| TRINITY_DN29306_c0_g1 | BIP ASPNG | bipA | Unregulated at 85% and 100% ERH | 0 |
| TRINITY_DN14038_c0_g1 | GPA1_EMENI | fadA | Unregulated at 85% and 100% ERH | 0 |
| TRINITY_DN58717_c0_g1 | NDUS8_NEUCR | nuo21.3c | Unregulated at 85% and 100% ERH | 0 |
| TRINITY_DN9867_c0_g2 | CAPZB_ASPFU | cap2 | Unregulated at 85% ERH | 0 |
| TRINITY_DN19748_c0_g1 | CATB_ASPOR | catB | Unregulated at 85% ERH | 0 |
| TRINITY_DN2314_c0_g2 | CHSA EMENI | chsA | Unregulated at 85% ERH | 0 |
| TRINITY_DN4298_c0_g1 | GPAA_ASPFC | gpaA | Unregulated at 85% ERH | 0 |
| TRINITY_DN10244_c0_g1 | DYHC_EMENI | nudA | Unregulated at 85% ERH | 0 |
| TRINITY_DN13764_c0_g1 | RHOC EMENI | rhoC | Unregulated at 85% ERH | 0 |
| TRINITY_DN4794_c0_g1 | STE12_EMENI | steA | Unregulated at 85% ERH | 0 |
| TRINITY_DN2841_c0_g2 | CAN1C_EMENI | candA-C | Unregulated at 85% ERH | 0 |
| TRINITY_DN21893_c0_g2 | CAN1N EMENI | candA-N | Unregulated at 85% ERH | 0 |
| TRINITY_DN1725_c0_g1 | CCG8_NEUCR | ccg-8 | Unregulated at 85% ERH | 0 |
| TRINITY_DN6916_c0_g1 | DOP1 EMENI | dop1 | Unregulated at 85% ERH | 0 |
| TRINITY_DN11446_c0_g1 | FLBA_EMENI | flbA | Unregulated at 85% ERH | 0 |
| TRINITY_DN219195_c0_g1 | FLUG_EMENI | fluG | Unregulated at 85% ERH | 0 |
| TRINITY_DN16965_c0_g1 | LAEA COCH5 | laeA | Unregulated at 85% ERH | 0 |
| TRINITY_DN15642_c0_g1 | DYL1_EMENI | nudG | Unregulated at 85% ERH | 0 |
| TRINITY_DN11172_c0_g2 | SIDH_ASPFU | sidH | Unregulated at 85% ERH | 0 |
| TRINITY_DN48838_c0_g1 | CHSC EMENI | chsC | Unregulated at 85% ERH | 0 |
| TRINITY_DN35531_c0_g1 | GRRA_EMENI | grrA | Unregulated at 85% ERH | 0 |
| TRINITY_DN10742_c0_g1 | HYMA_EMENI | hymA | Unregulated at 85% ERH | 0 |
| TRINITY_DN3657_c0_g1 | SIDC_ASPFU | sidC | Unregulated at 85% ERH | 0 |
| TRINITY_DN22023_c0_g1 | DYNA_NEUCR | ro-3 | Unregulated at 85% ERH | 0 |
| TRINITY_DN22889_c0_g3 | CDC45 SCHPO | sna41 | Unregulated at 85% ERH | 0 |
| TRINITY_DN290926_c0_g1 | ABAA_ASPFU | abaA | Unregulated at 85% ERH | 0 |
| TRINITY_DN30562_c0_g6 | SIDA_ASPFU | sidA | Unregulated at 85% ERH | 0 |
| TRINITY_DN23143_c0_g1 | STCC EMENI | stcC | Unregulated at 85% ERH | 0 |
| TRINITY_DN59728_c0_g3 | TCSA_EMENI | tcsA | Unregulated at 85% ERH | 0 |
| TRINITY_DN15743_c0_g2 | PPOA_EMENI | ppoA | Unregulated at 85% ERH | 0 |

| | Number of sites | Number of sites | | | |
|---|---|---|---|---|---|
| | the gene was | the gene was | | | |
| | expressed at | expressed at | | Fdr-adjusted p- | RH comparison (used for |
| Trinity Gene | 85% | 100% | Log2FC | value | log2FC) |
| TRINITY_DN8671_c0_g1 | 9 | 9 | 7.74 | 3.25E−10 | 100% vs 50% |
| TRINITY_DN60428_c0_g1 | 0 | 9 | 11.46 | 2.38E−20 | 100% vs 85% |
| TRINITY_DN22030_c0_g1 | 1 | 9 | 9.3 | 3.28E−14 | 100% vs 85% |
| TRINITY_DN8361_c0_g1 | 0 | 9 | 12.11 | 4.52E−25 | 100% vs 85% |
| TRINITY_DN5894_c0_g2 | 4 | 9 | 5.12 | 1.84E−07 | 100% vs 85% |
| TRINITY_DN1656_c0_g1 | 2 | 9 | 8.68 | 7.18E−08 | 100% vs 50% |
| TRINITY_DN372_c0_g1 | 0 | 9 | 12 | 3.54E−17 | 100% vs 85% |
| TRINITY_DN13900_c0_2g | 0 | 8 | 12.34 | 7.15E−13 | 100% vs 85% |
| TRINITY_DN13993_c0_g1 | 0 | 8 | 11.9 | 1.07E−16 | 100% vs 85% |
| TRINITY_DN8726_c0_g1 | 2 | 8 | 7.18 | 1.39E−05 | 100% vs 50% |
| TRINITY_DN34293_c0_g1 | 0 | 8 | 9.04 | 9.15E−14 | 100% vs 85% |
| TRINITY_DN46210_c0_g1 | 2 | 8 | 5.37 | 1.22E−05 | 100% vs 85% |
| TRINITY_DN18611_c0_g1 | 0 | 8 | 11.91 | 8.06E−19 | 100% vs 85% |
| TRINITY_DN8019_c0_g1 | 1 | 8 | 8.47 | 1.39E−13 | 100% vs 85% |
| TRINITY_DN2023_c0_g1 | 0 | 8 | 11 | 2.51E−15 | 100% vs 85% |
| TRINITY_DN1053_c0_g4 | 0 | 8 | 8.33 | 1.14E−10 | 100% vs 85% |
| TRINITY_DN2690_c0_g1 | 0 | 8 | 10.35 | 1.02E−12 | 100% vs 85% |
| TRINITY_DN285_c0_g2 | 0 | 8 | 8.91 | 8.37E−12 | 100% vs 85% |
| TRINITY_DN3379_c0_g1 | 2 | 7 | 7.15 | 1.82E−08 | 100% vs 85% |
| TRINITY_DN6303_c0_g1 | 0 | 7 | 10.42 | 5.16E−11 | 100% vs 85% |
| TRINITY_DN19513_c0_g2 | 1 | 7 | 8.82 | 1.22E−04 | 100% vs 85% |
| TRINITY_DN1945_c0_g1 | 1 | 7 | 9.39 | 6.82E−06 | 100% vs 50% |
| TRINITY_DN166_c0_g4 | 0 | 7 | 8.97 | 2.03E−11 | 100% vs 85% |
| TRINITY_DN5789_c0_g1 | 0 | 7 | 8.54 | 1.77E−12 | 100% vs 85% |
| TRINITY_DN51057_c0_g1 | 0 | 6 | 9.29 | 2.02E−08 | 100% vs 85% |
| TRINITY_DN13539_c0_g1 | 1 | 5 | 5.4 | 6.36E−04 | 100% vs 85% |
| TRINITY_DN17345_c0_g2 | 0 | 5 | 8.97 | 7.45E−05 | 100% vs 85% |
| TRINITY_DN35090_c0_g1 | 0 | 4 | 25.69 | 1.28E−27 | 100% vs 85% |
| TRINITY_DN81095_c0_g1 | 0 | 3 | 7.39 | 3.79E−06 | 100% vs 85% |
| TRINITY_DN3575_c0_g1 | 9 | 9 | 50.91 | 1.72E−05 | 100% vs 50% |
| TRINITY_DN11730_c0_g1 | 5 | 9 | 167.76 | 5.63E−11 | 100% vs 50% |
| TRINITY_DN12451_c0_g1 | 5 | 8 | 53.19 | 4.09E−08 | 100% vs 50% |
| TRINITY_DN10372_c0_g1 | 8 | 8 | 30.39 | 7.49E−07 | 100% vs 50% |
| TRINITY_DN20577_c0_g2 | 7 | 8 | 53.98 | 1.09E−07 | 100% vs 50% |
| TRINITY_DN8240_c0_g2 | 8 | 8 | 9.09 | 8.67E−05 | 100% vs 50% |
| TRINITY_DN7974_c0_g2 | 3 | 7 | 29.96 | 1.46E−05 | 100% vs 50% |
| TRINITY_DN5762_c0_g1 | 4 | 7 | 71.87 | 8.94E−08 | 100% vs 50% |
| TRINITY_DN29306_c0_g1 | 8 | 6 | 23.19 | 7.44E−05 | 100% vs 50% |
| TRINITY_DN14038_c0_g1 | 3 | 6 | 17.42 | 3.31E−05 | 100% vs 50% |
| TRINITY_DN58717_c0_g1 | 3 | 5 | 20.43 | 1.49E−05 | 100% vs 50% |
| TRINITY_DN9867_c0_g2 | 9 | 6 | 8.92 | 8.05E−13 | 85% vs 50% |
| TRINITY_DN19748_c0_g1 | 9 | 0 | 12.48 | 4.38E−22 | 85% vs 50% |
| TRINITY_DN2314_c0_g2 | 9 | 1 | 9.16 | 9.33E−11 | 85% vs 50% |
| TRINITY_DN4298_c0_g1 | 9 | 9 | 10.18 | 1.92E−22 | 85% vs 50% |
| TRINITY_DN10244_c0_g1 | 9 | 4 | 11.5 | 1.02E−16 | 85% vs 50% |
| TRINITY_DN13764_c0_g1 | 9 | 1 | 9.56 | 8.36E−15 | 85% vs 50% |
| TRINITY_DN4794_c0_g1 | 9 | 0 | 11.33 | 5.63E−18 | 85% vs 50% |
| TRINITY_DN2841_c0_g2 | 8 | 1 | 10.13 | 1.09E−12 | 85% vs 50% |
| TRINITY_DN21893_c0_g2 | 8 | 1 | 8.63 | 2.81E−10 | 85% vs 50% |
| TRINITY_DN1725_c0_g1 | 8 | 1 | 11.01 | 5.41E−14 | 85% vs 50% |
| TRINITY_DN6916_c0_g1 | 8 | 3 | 11.59 | 1.33E−13 | 85% vs 50% |
| TRINITY_DN11446_c0_g1 | 8 | 1 | 8.69 | 1.74E−11 | 85% vs 50% |
| TRINITY_DN219195_c0_g1 | 8 | 1 | 10.76 | 4.71E−12 | 85% vs 50% |
| TRINITY_DN16965_c0_g1 | 8 | 1 | 11.65 | 1.94E−16 | 85% vs 50% |
| TRINITY_DN15642_c0_g1 | 8 | 3 | 11.04 | 1.56E−12 | 85% vs 50% |
| TRINITY_DN11172_c0_g2 | 8 | 1 | 11.03 | 2.38E−15 | 85% vs 50% |
| TRINITY_DN48838_c0_g1 | 7 | 1 | 8.75 | 2.99E−09 | 85% vs 50% |
| TRINITY_DN35531_c0_g1 | 7 | 1 | 9.23 | 7.81E−10 | 85% vs 50% |
| TRINITY_DN10742_c0_g1 | 7 | 1 | 9.83 | 2.18E−09 | 85% vs 50% |
| TRINITY_DN3657_c0_g1 | 7 | 1 | 8.43 | 2.63E−10 | 85% vs 50% |
| TRINITY_DN22023_c0_g1 | 5 | 0 | 7.96 | 8.91E−06 | 85% vs 50% |
| TRINITY_DN22889_c0_g3 | 5 | 1 | 8.01 | 1.72E−06 | 85% vs 50% |
| TRINITY_DN290926_c0_g1 | 4 | 0 | 6.89 | 1.35E−04 | 85% vs 50% |
| TRINITY_DN30562_c0_g6 | 4 | 0 | 7.91 | 1.80E−05 | 85% vs 50% |
| TRINITY_DN23143_c0_g1 | 4 | 0 | 8.78 | 4.18E−06 | 85% vs 50% |
| TRINITY_DN59728_c0_g3 | 4 | 2 | 5.51 | 3.83E−04 | 85% vs 50% |
| TRINITY_DN15743_c0_g2 | 3 | 0 | 6.72 | 4.08E−04 | 85% vs 50% |

### Example 6

Gene expression was compared in house dust incubated at 50%, 85%, and 100% equilibrium relative humidity levels. The genes that are the most highly upregulated are the ones that are the most likely to serve as useful indicators of mold growth in homes. These genes (RNA) could be used directly to measure mold growth in homes, or their protein products may also be used. All of these genes are associated with fungal growth.

The samples used were collected from 9 different homes around the United States. Six of the homes were geographically distributed and the remaining three were from Columbus, OH. This will help determine how geographic distribution effects differ.

TABLE 3
GO enrichment: FDR < 0.05; P-value < 0.001, log2foldchange > 2.
100 vs 85, 100% upregulated:

| | | | | log2fold |
|---|---|---|---|---|
| # | Uniprot gene | Gene | Uniprot protein | change |
| 1 | ABR1_ASPFU | ABR1 | Multicopper oxidase abr1 (EC 1.—.—.—) (Conidial | 103.7851067 |
| | | | pigment biosynthesis oxidase abr1) | |
| 2 | AL15_ASPFU | AL15 | Allergen Asp f 15 (Allergen Asp f 13) (allergen | 87.80041336 |
| | | | Asp f 15) | |
| 3 | ALL4_ASPFU | ALL4 | Allergen Asp f 4 (allergen Asp f 4) | 95.93729494 |
| 4 | ALP2_ASPFU | ALP2 | Alkaline protease 2 (ALP2) (EC 3.4.21.63) | 88.15191659 |
| | | | (Autophagic serine protease alp2) (allergen Asp f 18) | |
| 5 | AP1_EMENI | AP1 | AP-1-like transcription factor napA | 59.55189882 |
| 6 | APSA_EMENI | APSA | Anucleate primary sterigmata protein A | 77.22745034 |
| 7 | ARP1_ASPFU | ARP1 | Scytalone dehydratase arp1 (EC 4.2.1.94) | 296.3110291 |
| | | | (Conidial pigment biosynthesis oxidase arp1) | |
| 8 | CALA_EMENI | CALA | Secreted thaumatin-like protein calA (CetA-like | 15149.93386 |
| | | | protein A) | |
| 9 | CAN1C_EMENI | CAN1C | Cullin-associated NEDD8-dissociated protein 1, | 34.41129997 |
| | | | C-terminal part (Cullin-associated and | |
| | | | neddylation-dissociated protein 1, C-terminal | |
| | | | part) | |
| 10 | CAN1N_EMENI | CAN1N | Cullin-associated NEDD8-dissociated protein 1, | 45.86490019 |
| | | | N-terminal part (Cullin-associated and | |
| | | | neddylation-dissociated protein 1, N-terminal | |
| | | | part) | |
| 11 | CAT1_NEUCR | CAT1 | Catalase-1 (EC 1.11.1.6) | 39.6110021 |
| 12 | CAT3_NEUCR | CAT3 | Catalase-3 (EC 1.11.1.6) | 30.31222317 |
| 13 | CATA_ASPFU | CATA | Catalase A (EC 1.11.1.6) (Fast catalase) | 15.70813056 |
| 14 | CETA_EMENI | CETA | Secreted thaumatin-like protein cetA (Conidial- | 115.022758 |
| | | | enriched transcript protein A) | |
| 15 | CFMA_ASPFU | CFMA | GPI-anchored hemophore cfmA (GPI-anchored | 509.6405058 |
| | | | CFEM domain protein A) | |
| 16 | CHIA1_EMENI | CHIA1 | Endochitinase A (EC 3.2.1.14) (Chitinase A) | 22.13741536 |
| 17 | CHS1_NEUCR | CHS1 | Chitin synthase 1 (EC 2.4.1.16) (Chitin-UDP | 35.7053251 |
| | | | acetyl-glucosaminyl transferase 1) (Class-III | |
| | | | chitin synthase 3) | |
| 18 | CHSD_ASPFU | CHSD | Chitin synthase D (EC 2.4.1.16) (Chitin-UDP | 47.12940293 |
| | | | acetyl-glucosaminyl transferase D) (Class-VI | |
| | | | chitin synthase D) | |
| 19 | CHSG_ASPFU | CHSG | Chitin synthase G (EC 2.4.1.16) (Chitin-UDP | 21.65629861 |
| | | | acetyl-glucosaminyl transferase G) (Class-III | |
| | | | chitin synthase G) | |
| 20 | CON10_NEUCR | CON10 | Conidiation-specific protein 10 | 163.9164276 |
| 21 | CON6_NEUCR | CON6 | Conidiation-specific protein 6 | 338.7940477 |
| 22 | CON8_NEUCR | CON8 | Conidiation-specific protein 8 | 88.04340118 |
| 23 | CRZA_ASPFU | CRZA | C2H2 finger domain transcription factor crzA | 25.93793161 |
| 24 | DEWA_EMENI | DEWA | Spore-wall fungal hydrophobin dewA | 16.81905494 |
| 25 | DOP1_EMENI | DOP1 | Protein dopey | 823.465505 |
| 26 | DYL1_EMENI | DYL1 | Dynein light chain, cytoplasmic (8 kDa | 12.35143462 |
| | | | cytoplasmic dynein light chain) | |
| 27 | ECM33_ASPFU | ECM33 | Protein ecm33 | 69.55662103 |
| 28 | ENO_EMENI | ENO | Enolase (EC 4.2.1.11) (2-phospho-D-glycerate | 112.295492 |
| | | | hydro-lyase) (2-phosphoglycerate dehydratase) | |
| 29 | FLBA_EMENI | FLBA | Developmental regulator flbA | 24.76622898 |
| 30 | FLUG_EMENI | FLUG | Protein fluG | 28.56110248 |
| 31 | GEL4_ASPFU | GEL4 | 1,3-beta-glucanosyltransferase gel4 (EC 2.4.1.—) | 17.27554597 |
| | | | (Glucan elongating glucanosyltransferase 4) | |
| 32 | GPA1_EMENI | GPA1 | Guanine nucleotide-binding protein subunit | 66.66180195 |
| | | | alpha | |
| 33 | GPA3_NEUCR | GPA3 | Guanine nucleotide-binding protein alpha-3 | 62.15910606 |
| | | | subunit (GP3-alpha) | |
| 34 | GRRA_EMENI | GRRA | SCF E3 ubiquitin ligase complex F-box protein | 75.05549585 |
| | | | grrA (F-box and leucine-rich repeat protein | |
| | | | grrA) (F-box/LRR-repeat protein grrA) (SCF | |
| | | | substrate adapter protein grrA) | |
| 35 | HSP30_EMENI | HSP30 | 30 kDa heat shock protein | 43.80034932 |
| 36 | HSP70_EMENI | HSP70 | Heat shock 70 kDa protein (HSP70) | 39.32933465 |
| 37 | HYMA_EMENI | HYMA | Conidiophore development protein hymA | 37.26879107 |
| 38 | KAPR_ASPFU | KAPR | CAMP-dependent protein kinase regulatory | 14.95352835 |
| | | | subunit (PKA regulatory subunit) | |
| 39 | KATG_EMENI | KATG | Catalase-peroxidase (CP) (EC 1.11.1.21) | 262.2764155 |
| | | | (Peroxidase/catalase) | |
| 40 | LAEA_PENRW | LAEA | Secondary metabolism regulator laeA | 206.7533945 |
| | | | (Methyltransferase laeA) (EC 2.1.1.—) (Velvet | |
| | | | complex subunit laeA) | |
| 41 | MTLD_EMENI | MTLD | Mannitol-1-phosphate 5-dehydrogenase | 11.91234659 |
| | | | (M1PDH) (MPD) (MPDH) (EC 1.1.1.17) | |
| 42 | NADA_NEUCR | NADA | Conidial surface nicotinamide adenine | 121.2115822 |
| | | | dinucleotide glycohydrolase (NAD(P)ase) | |
| | | | (NADase) (Diphosphopyridine nucleotidase) | |
| | | | (DPNase) (NAD(+) hydrolase) (EC 3.2.2.5) | |
| | | | (NADP(+) hydrolase) (EC 3.2.2.—) | |
| 43 | NRC2_NEUCR | NRC2 | Serine/threonine-protein kinase nrc-2 (EC | 8.054008269 |
| | | | 2.7.11.1) (Non-repressible conidiation protein 2) | |
| 44 | PHIA_EMENI | PHIA | Cell wall protein phiA (Bafilomycin-induced | 27.20435896 |
| | | | protein B) (Phialide development protein A) | |
| 45 | PP1_EMENI | PP1 | Serine/threonine-protein phosphatase PP1 (EC | 22.75202201 |
| | | | 3.1.3.16) | |
| 46 | RCO1_NEUCR | RCO1 | Transcriptional repressor rco-1 | 69.21897389 |
| 47 | RNY1_ASPFU | RNY1 | Ribonuclease T2-like (RNase T2-like) (EC | 22.99685759 |
| | | | 4.6.1.19) | |
| 48 | RODL_ASPFU | RODL | Hydrophobin (Rodlet protein) | 17.99550977 |
| 49 | SODC_EMENI | SODC | Superoxide dismutase [Cu—Zn] (EC 1.15.1.1) | 36.28915432 |
| 50 | SUN1_ASPFU | SUN1 | Secreted beta-glucosidase sun1 (EC 3.2.1.—) | 26.79201912 |
| 51 | TBA2_EMENI | TBA2 | Tubulin alpha-2 chain (EC 3.6.5.—) | 171.3219323 |
| 52 | TCSA_EMENI | TCSA | Two-component system protein A (EC 2.7.13.3) | 3800.770164 |
| 53 | TPIS_EMENI | TPIS | Triosephosphate isomerase (TIM) (EC 5.3.1.1) | 130.3226867 |
| | | | (Triose-phosphate isomerase) | |
| 54 | TPS1_EMENI | TPS1 | Alpha, alpha-trehalose-phosphate synthase | 22.63866963 |
| | | | [UDP-forming] (EC 2.4.1.15) (Trehalose-6- | |
| | | | phosphate synthase) (UDP-glucose- | |
| | | | glucosephosphate glucosyltransferase) | |
| 55 | TREA_EMENI | TREA | Acid trehalase (EC 3.2.1.28) (Alpha, alpha- | 66.69086398 |
| | | | trehalase) (Alpha, alpha-trehalose | |
| | | | glucohydrolase) | |
| 56 | UBC2_NEUCR | UBC2 | Ubiquitin-conjugating enzyme E2 2 (EC | 4.327167947 |
| | | | 2.3.2.23) (E2 ubiquitin-conjugating enzyme 2) | |
| | | | (Mutagen-sensitive protein 8) (Ubiquitin carrier | |
| | | | protein ubc2) (Ubiquitin-protein ligase ubc2) | |
| 57 | VELB_PENRW | VELB | Velvet complex subunit B | 152.011901 |
| 58 | VOSA_EMENI | VOSA | Spore development regulator vosA (Viability of | 53.78059707 |
| | | | spores protein A) | |
| 59 | WA_EMENI | WA | Conidial yellow pigment biosynthesis polyketide | 30.28902057 |
| | | | synthase (PKS) (EC 2.3.1.—) | |
| 60 | MELA_PENEN | MELA | Conidial yellow pigment biosynthesis polyketide | 87.57259444 |
| | | | synthase melA (PKS melA) (EC 2.3.1.—) | |
| 61 | WETA_PEND2 | WETA | Developmental regulatory protein wetA | 62.73797378 |
| 62 | BRLA_PENCA | BRLA | C2H2 type master regulator of conidiophore | 293.5925249 |
| | | | development brlA | |
| 63 | TBA1_EMENI | TBA1 | Tubulin alpha-1 chain (EC 3.6.5.—) | 16.93173927 |
| 64 | TBG_NEUCR | TBG | Tubulin gamma chain (Gamma-tubulin) | 8.105732666 |
| 65 | SIDL_ASPFU | SIDL | Acyltransferase sidL (EC 2.3.1.—) (Siderophore | 117.8830625 |
| | | | biosynthesis protein L) | |
| 66 | SIDI_ASPFU | SIDI | Acyl-CoA ligase sidI (EC 6.2.1.—) (Siderophore | 19.39616967 |
| | | | biosynthesis protein I) | |
| 67 | EF2_NEUCR | EF2 | Elongation factor 2 (EF-2) (Colonial | 85.5892763 |
| | | | temperature-sensitive 3) | |
| 68 | SIDH_ASPFU | SIDH | Mevalonyl-coenzyme A hydratase sidH (EC | 206.578626 |
| | | | 4.2.1.—) (Siderophore biosynthesis protein H) | |
| 69 | AYG1_ASPFU | AYG1 | Heptaketide hydrolyase ayg1 (EC 3.7.1.—) | 8.061501532 |
| | | | (Conidial pigment biosynthesis protein ayg1) | |
| 70 | STCC_EMENI | STCC | Peroxidase stcC (EC 1.11.1.—) (Sterigmatocystin | 13.54790161 |
| | | | biosynthesis cluster protein C) | |
| 71 | HEX1_NEUCR | HEX1 | Woronin body major protein | 614.4498717 |
| 72 | HEXA_ASPFU | HEXA | Woronin body major protein hexA | 10.63341923 |
| 73 | RHOA_EMENI | RHOA | GTP-binding protein rhoA (Rho1 protein | 86.9276875 |
| | | | homolog) | |
| 74 | PRX5_ASPFU | PRX5 | Peroxiredoxin Asp f3 (Prx) (EC 1.11.1.24) | 8.092400284 |
| | | | (Thioredoxin peroxidase) (TPx) (Thioredoxin- | |
| | | | dependent peroxiredoxin) (allergen Asp f 3) | |
| 75 | ARP2_ASPFU | ARP2 | Hydroxynaphthalene reductase arp2 (EC 1.1.—.—) | 283.6591087 |
| | | | (Conidial pigment biosynthesis oxidase arp2) | |
| 76 | IVOB_EMENI | IVOB | N-acetyl-6-hydroxytryptophan oxidase ivoB | 140.3233423 |
| | | | (AHTase ivoB) (EC 1.14.—.—) (Ivory mutation- | |
| | | | related protein B) (Monophenol oxidase) | |
| 77 | EF1A_ASPOR | EF1A | Elongation factor 1-alpha (EF-1-alpha) | 107.625383 |
| 78 | ABR2_ASPFU | ABR2 | Laccase abr2 (EC 1.10.3.—) (Conidial pigment | 266.2275184 |
| | | | biosynthesis oxidase abr2) | |
| 79 | STUA_ASPEN | STUA | Cell pattern formation-associated protein stuA | 132.9713391 |
| | | | (Stunted protein A) | |
| 80 | SEPH_ASPTN | SEPH | Cytokinesis protein sepH (EC 2.7.11.1) | 154.2163534 |
| | | | (Serine/threonine-protein kinase sepH) | |
| 81 | SEPA_EMENI | SEPA | Cytokinesis protein sepA (Forced expression | 1343.914178 |
| | | | inhibition of growth A) (Protein FH1/2) | |
| 82 | ASD4_NEUCR | ASD4 | GATA type zinc finger protein asd-4 (Ascus | 6.209581728 |
| | | | development protein 4) | |
| 83 | CRF1_ASPFU | CRF1 | Probable glycosidase crf1 (EC 3.2.—.—) (Crh-like | 32.14014702 |
| | | | protein 1) (allergen Asp f 9) | |
| 84 | ORYZ_ASPFU | ORYZ | Alkaline protease 1 (ALP) (EC 3.4.21.63) | 21.52145265 |
| | | | (Aspergillopeptidase B) (Aspergillus proteinase | |
| | | | B) (Elastase) (Elastinolytic serine proteinase) | |
| | | | (Oryzin) (allergen Asp f 13) | |
| 85 | QUTR_EMENI | QUTR | Quinate repressor protein | 19.13139287 |
| 86 | XYNC_EMENI | XYNC | Endo-1,4-beta-xylanase C (Xylanase C) (EC | 13.62848612 |
| | | | 3.2.1.8) (1,4-beta-D-xylan xylanohydrolase C) | |
| | | | (34 kDa xylanase) (Xylanase X34) | |
| 87 | ATNM_EMENI | ATNM | Fatty acid synthase beta subunit aflB (EC | 40.6965362 |
| | | | 2.3.1.86) (Aspercryptin biosynthesis cluster | |
| | | | protein M) [Includes: 3-hydroxyacyl-[acyl- | |
| | | | carrier-protein] dehydratase (EC 4.2.1.59); | |
| | | | Enoyl-[acyl-carrier-protein] reductase [NADH] | |
| | | | (EC 1.3.1.9); [Acyl-carrier-protein] | |
| | | | acetyltransferase (EC 2.3.1.38); [Acyl-carrier- | |
| | | | protein] malonyltransferase (EC 2.3.1.39); S- | |
| | | | acyl fatty acid synthase thioesterase (EC | |
| | | | 3.1.2.14)] | |
| 88 | CY1_NEUCR | CY1 | Cytochrome c1, heme protein, mitochondrial | 189.0863163 |
| | | | (EC 7.1.1.8) (Complex III subunit 4) (Complex | |
| | | | III subunit IV) (Cytochrome b-c1 complex | |
| | | | subunit 4) (Ubiquinol-cytochrome c | |
| | | | oxidoreductase complex cytochrome c1 subunit) | |
| | | | (Cytochrome c-1) (Ubiquinol-cytochrome c | |
| | | | reductase complex 31 kDa protein) | |
| 89 | LKHA4_ASPNC | LKHA4 | Leucine aminopeptidase 2 (EC 3.4.11.—) | 7.089953847 |
| | | | (Epoxide hydrolase) (EC 3.3.2.10) (Leukotriene | |
| | | | A-4 hydrolase homolog) (LTA-4 hydrolase) | |
| 90 | RLA1_PENBR | RLA1 | 60S acidic ribosomal protein P1 (allergen Pen b | 730.4333113 |
| | | | 26.0101) | |
| 91 | EF1B_PENCI | EF1B | Elongation factor 1-beta (EF-1-beta) (allergen | 464.8909257 |
| | | | Pen c 24) | |
| 92 | VRTJ_PENAE | VRTJ | Aldolase vrtJ (EC 4.1.2.—) (Viridicatumtoxin | 9.183207316 |
| | | | synthesis protein J) | |
| 93 | ALTA7_ALTAL | ALTA7 | Minor allergen Alt a 7 | 7.74 |
| 94 | NDUS2_NEUCR | NUO-49 | NADH-ubiquinone oxidoreductase 49 kDa subunit, | 5.12 |
| | | | mitochondrial | |

### GO Terms:

CC extracellular region
BP anatomical structure formation involved in morphogenesis
BP cell differentiation
BP sporulation
BP sporulation resulting in formation of a cellular spore
BP asexual sporulation
BP conidium formation
MF catalase activity
MF xylanase activity
MF P-type sodium transporter activity
MF ABC-type sodium transporter activity
BP methane metabolic process
MF alcohol oxidase activity
BP methanol metabolic process
BP response to farnesol

TABLE 4
GO enrichment: FDR < 0.05; P-value < 0.001, log2foldchange > 2
100 vs 50, 100% upregulated:

| | | Uniprot | | log2fold |
|---|---|---|---|---|
| # | Uniprot gene | gene | Uniprot protein | change |
| 2 | DEWA_EMENI | DEWA | Spore-wall fungal hydrophobin dewA | 2376.592 |
| 3 | WETA_EMENI | WETA | Developmental regulatory protein wetA | 29.83244 |
| 4 | CON6_NEUCR | CON6 | Conidiation-specific protein 6 | 3313.639 |
| 5 | RODL_ASPFU | RODL | Hydrophobin (Rodlet protein) | 165.8692 |
| 6 | DYL1_EMENI | DYL1 | Dynein light chain, cytoplasmic (8 kDa | 41.90496 |
| | | | cytoplasmic dynein light chain) | |
| 7 | VELB_PENRW | VELB | Velvet complex subunit B | 40.49207 |
| 8 | TPS1_EMENI | TPS1 | Alpha,alpha-trehalose-phosphate synthase | 4.73055 |
| | | | [UDP-forming] (EC 2.4.1.15) (Trehalose-6- | |
| | | | phosphate synthase) (UDP-glucose- | |
| | | | glucosephosphate glucosyltransferase) | |
| 9 | AP1_EMENI | AP1 | AP-1-like transcription factor napA | 29.31585 |
| 10 | WA_EMENI | WA | Conidial yellow pigment biosynthesis | 141.7245 |
| | | | polyketide synthase (PKS) (EC 2.3.1.—) | |
| 11 | ARP1_ASPFU | ARP1 | Scytalone dehydratase arp1 (EC 4.2.1.94) | 155.048 |
| | | | (Conidial pigment biosynthesis oxidase arp1) | |
| 13 | CON10_NEUCR | CON10 | Conidiation-specific protein 10 | 2499.892 |
| 14 | PP1_NEUCR | PP1 | Serine/threonine-protein phosphatase PP1 (EC | 37.75065 |
| | | | 3.1.3.16) (Phosphoprotein phosphatase 1) | |
| 15 | CAN1C_EMENI | CAN1C | Cullin-associated NEDD8-dissociated protein 1, | 15.72045 |
| | | | C-terminal part (Cullin-associated and | |
| | | | neddylation-dissociated protein 1, C-terminal | |
| | | | part) | |
| 16 | RCO1_NEUCR | RC01 | Transcriptional repressor rco-1 | 70.50166 |
| 17 | VOSA_PENRW | VOSA | Spore development regulator vosA | 51.10854 |
| 18 | TCSA_EMENI | TCSA | Two-component system protein A (EC 2.7.13.3) | 34.06002 |
| 19 | UBC2_EMENI | UBC2 | Ubiquitin-conjugating enzyme E2 2 (EC | 25.01475 |
| | | | 2.3.2.23) (E2 ubiquitin-conjugating enzyme 2) | |
| | | | (UV sensitivity protein J) (Ubiquitin carrier | |
| | | | protein ubc2) (Ubiquitin-protein ligase ubc2) | |
| 20 | NRC2_NEUCR | NRC2 | Serine/threonine-protein kinase nrc-2 (EC | 11.59366 |
| | | | 2.7.11.1) (Non-repressible conidiation protein 2) | |
| 21 | ALP2_ASPFU | ALP2 | Alkaline protease 2 (ALP2) (EC 3.4.21.63) | 71.17394 |
| | | | (Autophagic serine protease alp2) (allergen Asp f 18) | |
| 22 | GPA3_NEUCR | GPA3 | Guanine nucleotide-binding protein alpha-3 | 34.21216 |
| | | | subunit (GP3-alpha) | |
| 23 | GRRA_EMENI | GRRA | SCF E3 ubiquitin ligase complex F-box protein | 20.47834 |
| | | | grrA (F-box and leucine-rich repeat protein | |
| | | | grrA) (F-box/LRR-repeat protein grrA) (SCF | |
| | | | substrate adapter protein grrA) | |
| 24 | CAN1N_EMENI | CAN1N | Cullin-associated NEDD8-dissociated protein 1, | 22.23194 |
| | | | N-terminal part (Cullin-associated and | |
| | | | neddylation-dissociated protein 1, N-terminal | |
| | | | part) | |
| 25 | CATA_ASPFU | CATA | Catalase A (EC 1.11.1.6) (Fast catalase) | 673.3545 |
| 27 | KAPR_ASPFU | KAPR | CAMP-dependent protein kinase regulatory | 124.2351 |
| | | | subunit (PKA regulatory subunit) | |
| 29 | TPIS_EMENI | TPIS | Triosephosphate isomerase (TIM) (EC 5.3.1.1) | 167.7596 |
| | | | (Triose-phosphate isomerase) | |
| 30 | HSP70_EMENI | HSP70 | Heat shock 70 kDa protein (HSP70) | 978.3023 |
| 31 | HSP30_EMENI | HSP30 | 30 kDa heat shock protein | 15.15134 |
| 32 | MDM10_EMENI | MDM10 | Mitochondrial distribution and morphology | 71.87014 |
| | | | protein 10 (Mitochondrial inheritance | |
| | | | component mdm 10) | |
| 33 | ENO_EMENI | ENO | Enolase (EC 4.2.1.11) (2-phospho-D-glycerate | 55.23125 |
| | | | hydro-lyase) (2-phosphoglycerate dehydratase) | |
| 34 | CRZA_ASPFU | CRZA | C2H2 finger domain transcription factor crzA | 30.38697 |
| 35 | GPA1_EMENI | GPA1 | Guanine nucleotide-binding protein subunit | 17.41959 |
| | | | alpha | |
| 36 | FLBA_EMENI | FLBA | Developmental regulator flbA | 13.21341 |
| 37 | MPG1_EMENI | MPG1 | Mannose-1-phosphate guanyltransferase (EC | 34.33718 |
| | | | 2.7.7.13) (GDP-mannose pyrophosphorylase) | |
| | | | (GTP-mannose-1-phosphate | |
| | | | guanylyltransferase) | |
| 38 | APSA_EMENI | APSA | Anucleate primary sterigmata protein A | 15.99781 |
| 39 | CHSD_ASPFU | CHSD | Chitin synthase D (EC 2.4.1.16) (Chitin-UDP | 21.11069 |
| | | | acetyl-glucosaminyl transferase D) (Class-VI | |
| | | | chitin synthase D) | |
| 40 | ACH1_NEUCR | ACH1 | Acetyl-CoA hydrolase (EC 3.1.2.1) (Acetate | 45.78404 |
| | | | utilization protein) (Acetyl-CoA deacylase) | |
| | | | (Acetyl-CoA acylase) | |
| 41 | ATFB_ASPFU | ATFB | Basic leucine zipper (bZIP) transcription factor | 575.8772 |
| | | | atfB | |
| 42 | GEL1_ASPFU | GEL1 | 1,3-beta-glucanosyltransferase gel1 (EC 2.4.1.—) | 25.79039 |
| | | | (Glucan elongating glucanosyltransferase 1) | |
| 43 | GEL2_ASPFU | GEL2 | 1,3-beta-glucanosyltransferase gel2 (EC 2.4.1.—) | 14.52525 |
| | | | (Glucan elongating glucanosyltransferase 2) | |
| 44 | CCG6_NEUCR | CCG6 | Clock-controlled protein 6 | 678.8133 |
| 45 | HSP90_ASPFU | HSP90 | Heat shock protein 90 (65 kDa IgE-binding | 373.0016 |
| | | | protein) (Heat shock protein hsp1) (allergen Asp f 12) | |
| 46 | ECM33_ASPFU | ECM33 | Protein ecm33 | 53.97603 |
| 47 | CRF1_ASPFU | CRF1 | Probable glycosidase crf1 (EC 3.2.—.—) (Crh-like | 42.9854 |
| | | | protein 1) (allergen Asp f 9) | |
| 48 | EGLC_EMENI | EGLC | Probable glucan endo-1,3-beta-glucosidase eglC | 164.5627 |
| | | | (EC 3.2.1.39) (Endo-1,3-beta-glucanase eglC) | |
| | | | (Laminarinase eglC) | |
| 49 | STE20_EMENI | STE20 | Serine/threonine-protein kinase ste20 (EC | 17.55047 |
| | | | 2.7.11.1) | |
| 50 | NDUS8_NEUCR | NUO21.3C | NADH-ubiquinone oxidoreductase 23 kDa | 20.43 |
| | | | subunit, mitochondrial | |

### GO Terms:

BP cellular developmental process
BP cell differentiation
BP anatomical structure formation involved in morphogenesis
BP sporulation
BP sporulation resulting in formation of a cellular spore
CC fungal-type cell wall
BP asexual reproduction
BP asexual sporulation
BP conidium formation
BP regulation of aflatoxin biosynthetic process
BP positive regulation of aflatoxin biosynthetic process
BP asexual spore wall assembly
BP trehalose metabolism in response to stress
BP response to farnesol

TABLE 5
GO enrichment: FDR < 0.05; P-value < 0.001, log2foldchange > 2.
50vs85, 85% Upregulated:

| | | | | log2fold |
|---|---|---|---|---|
| # | Uniprot gene | Gene | Uniprot protein | change |
| 1 | ALP2_ASPFU | ALP2 | Alkaline protease 2 (ALP2) (EC 3.4.21.63) | 367.5895 |
| | | | (Autophagic serine protease alp2) (allergen Asp f 18) | |
| 2 | FLUG_EMENI | FLUG | Protein fluG | 9.105208 |
| 3 | CRZA_ASPFU | CRZA | C2H2 finger domain transcription factor crzA | 32.92718 |
| 4 | HYMA_EMENI | HYMA | Conidiophore development protein hymA | 66.59954 |
| 5 | FLBA_EMENI | FLBA | Developmental regulator flbA | 84.00904 |
| 6 | DOP1_EMENI | DOP1 | Protein dopey | 34.173 |
| 8 | CHSD_EMENI | CHSD | Chitin synthase D (EC 2.4.1.16) (Chitin-UDP acetyl- | 42.8871 |
| | | | glucosaminyl transferase D) (Class-V chitin synthase D) | |
| 9 | KAPR_ASPFU | KAPR | CAMP-dependent protein kinase regulatory subunit | 21.94404 |
| | | | (PKA regulatory subunit) | |
| 10 | TBA2_EMENI | TBA2 | Tubulin alpha-2 chain (EC 3.6.5.—) | 966.1438 |
| 11 | GPA1_EMENI | GPA1 | Guanine nucleotide-binding protein subunit alpha | 22.86247 |
| 12 | APSA_EMENI | APSA | Anucleate primary sterigmata protein A | 10.16529 |
| 13 | DYL1_EMENI | DYL1 | Dynein light chain, cytoplasmic (8 kDa cytoplasmic | 786.1241 |
| | | | dynein light chain) | |
| 14 | PPOA_EMENI | PPOA | Psi-producing oxygenase A (Fatty acid oxygenase | 124.7714 |
| | | | ppoA) [Includes: Linoleate 8R-lipoxygenase (EC | |
| | | | 1.13.11.60); 9,12-octadecadienoate 8-hydroperoxide | |
| | | | 8R-isomerase (EC 5.4.4.5)] | |
| 15 | CHSA_EMENI | CHSA | Chitin synthase A (EC 2.4.1.16) (Chitin-UDP acetyl- | 47.4182 |
| | | | glucosaminyl transferase A) (Class-II chitin synthase A) | |
| 16 | RCO1_NEUCR | RCO1 | Transcriptional repressor rco-1 | 176.1636 |
| 17 | PP1_EMENI | PP1 | Serine/threonine-protein phosphatase PP1 (EC | 6.754616 |
| | | | 3.1.3.16) | |
| 18 | VEA_ASPFU | VEA | Developmental and secondary metabolism regulator | 44.81746 |
| | | | veA (Velvet complex subunit A) | |
| 19 | GRRA_EMENI | GRRA | SCF E3 ubiquitin ligase complex F-box protein grrA | 96.32193 |
| | | | (F-box and leucine-rich repeat protein grrA) (F- | |
| | | | box/LRR-repeat protein grrA) (SCF substrate adapter | |
| | | | protein grrA) | |
| 20 | CAN1C_EMENI | CAN1C | Cullin-associated NEDD8-dissociated protein 1, C- | 78.79247 |
| | | | terminal part (Cullin-associated and neddylation- | |
| | | | dissociated protein 1, C-terminal part) | |
| 21 | CAN1N_EMENI | CAN1N | Cullin-associated NEDD8-dissociated protein 1, N- | 62.65315 |
| | | | terminal part (Cullin-associated and neddylation- | |
| | | | dissociated protein 1, N-terminal part) | |
| 22 | CHSC_EMENI | CHSC | Chitin synthase C (EC 2.4.1.16) (Chitin-UDP acetyl- | 69.98266 |
| | | | glucosaminyl transferase C) (Class-I chitin synthase C) | |
| 23 | TCSA_EMENI | TCSA | Two-component system protein A (EC 2.7.13.3) | 8.46742 |
| 24 | ABAA_ASPFU | ABAA | Conidiophore development regulator abaA | 8.689378 |
| 25 | CBF5_EMENI | CBF5 | H/ACA ribonucleoprotein complex subunit CBF5 (EC | 5.675772 |
| | | | 5.4.99.—) (Centromere-binding factor 5) (H/ACA | |
| | | | snoRNP protein CBF5) (Small nucleolar RNP protein | |
| | | | CBF5) (rRNA pseudouridine synthase) | |
| 26 | UBC2_ASPFU | UBC2 | Ubiquitin-conjugating enzyme E2 2 (EC 2.3.2.23) (E2 | 421.9873 |
| | | | ubiquitin-conjugating enzyme 2) (Ubiquitin carrier | |
| | | | protein ubc2) (Ubiquitin-protein ligase ubc2) | |
| 27 | MRD1_ASPFU | MRD1 | Multiple RNA-binding domain-containing protein 1 | 68.72583 |
| 28 | DYHC_EMENI | DYHC | Dynein heavy chain, cytoplasmic (Dynein heavy | 211.6991 |
| | | | chain, cytosolic) (DYHC) | |
| 29 | CALM_EMENI | CALM | Calmodulin (CaM) | 16.24465 |
| 30 | CSN2_EMENI | CSN2 | COP9 signalosome complex subunit 2 (Signalosome | 84.7247 |
| | | | subunit 2) | |
| 31 | STE12_EMENI | STE12 | Transcription factor steA | 47.00275 |
| 32 | PLMT_EMENI | PLMT | Phosphatidyl-N-methylethanolamine N- | 296.8194 |
| | | | methyltransferase (EC 2.1.1.71) (Phospholipid | |
| | | | methyltransferase) (PLMT) | |
| 33 | BCP1_ASPFU | BCP1 | Protein bcp1 | 260.229 |
| 34 | TCTP_ASPFU | TCTP | Translationally-controlled tumor protein homolog | 170.6905 |
| | | | (TCTP) | |
| 35 | CSN7_EMENI | CSN7 | COP9 signalosome complex subunit 7 (CSN complex | 34.71311 |
| | | | subunit 7) | |
| 36 | COT1_NEUCR | COT1 | Serine/threonine-protein kinase cot-1 (EC 2.7.11.1) | 218.3901 |
| | | | (Colonial temperature-sensitive protein 1) | |
| 37 | STE20_ASPFU | STE20 | Serine/threonine-protein kinase ste20 (EC 2.7.11.1) | 70.36642 |
| 39 | GBLP_NEUCR | GBLP | Guanine nucleotide-binding protein subunit beta-like | 9137.342 |
| | | | protein (Cross-pathway control WD-repeat protein | |
| | | | cpc-2) | |
| 40 | HST4_EMENI | HST4 | NAD-dependent protein deacetylase hst4 (EC | 36.09289 |
| | | | 2.3.1.286) (Homologous to SIR2 protein 4) | |
| | | | (Regulatory protein SIR2 homolog 4) | |
| 41 | NDOR1_ASPFU | NDOR1 | NADPH-dependent diflavin oxidoreductase 1 (EC | 43.24289 |
| | | | 1.18.1.—) (NADPH-dependent FMN and FAD- | |
| | | | containing oxidoreductase) | |
| 42 | SEBA_ASPFU | SEBA | C2H2 finger domain transcription factor sebA (Stress | 210.6788 |
| | | | response element-binding protein A) | |
| 43 | CMR1_ASPFU | CMR1 | DNA damage-binding protein cmr1 | 20.06982 |
| 44 | MPKA_ASPFU | MPKA | Mitogen-activated protein kinase mpkA (MAP kinase | 12.57593 |
| | | | mpkA) (EC 2.7.11.24) | |
| 45 | RHOA_EMENI | RHOA | GTP-binding protein rhoA (Rhol protein homolog) | 23.37065 |
| 46 | MKK2_ASPFU | MKK2 | Mitogen-activated protein kinase kinae mkk2 | 201.4481 |
| | | | (MAPKK mkk2) (EC 2.7.11.24) (MEK mkk2) | |
| 47 | MESA_EMENI | MESA | Protein mesA | 122.1056 |
| 48 | PREG_NEUCR | PREG | Nuc-1 negative regulatory protein preg (Phosphatase | 124.7713 |
| | | | regulation protein) | |
| 49 | TFB5_ASPFU | TFB5 | General transcription and DNA repair factor IIH | 20.966 |
| | | | subunit tfb5 (TFIIH subunit tfb5) (RNA polymerase II | |
| | | | transcription factor B subunit 5) | |
| 50 | CDK1_EMENI | CDK1 | Cyclin-dependent kinase 1 (CDK1) (EC 2.7.11.22) | 17.47694 |
| | | | (Cell division control protein 2) (Cell division protein | |
| | | | kinase 1) (Never in mitosis protein X) | |
| 51 | CG21_EMENI | CG21 | G2/mitotic-specific cyclin-B | 184.3593 |
| 52 | TFB1_NEUCR | TFB1 | General transcription and DNA repair factor IIH | 18.45557 |
| | | | subunit tcf-29 (TFIIH subunit tcf-29) (RNA | |
| | | | polymerase II transcription factor B 73 kDa subunit) | |
| | | | (RNA polymerase II transcription factor B p73 | |
| | | | subunit) (RNA polymerase II transcription factor B | |
| | | | subunit 1) (Transcription factor-29) | |
| 53 | ATG11_ASPFU | ATG11 | Autophagy-related protein 11 | 27.67012 |
| 54 | BUR1_EMENI | BUR1 | Serine/threonine-protein kinase bur1 (EC 2.7.11.22) | 10.59784 |
| | | | (EC 2.7.11.23) (PITALRE-like kinase A) | |
| 55 | ATG13_EMENI | ATG13 | Autophagy-related protein 13 | 31.2232 |
| 56 | PTAB_ASPFU | PTAB | Transcriptional activator ptaB | 78.38127 |
| 58 | HEXA_ASPFU | HEXA | Woronin body major protein hexA | 83.0573 |
| 59 | HEX1_NEUCR | HEX1 | Woronin body major protein | 68.56953 |
| 60 | BIMC_EMENI | BIMC | Kinesin-like protein bimC | 48.73078 |
| 61 | KLPA_EMENI | KLPA | Kinesin-like protein klpA | 36.70711 |
| 62 | DYNA_NEUCR | DYNA | Dynactin, 150 kDa isoform (150 kDa dynein- | 69.91066 |
| | | | associated polypeptide) (DAP-150) (DP-150) (p150- | |
| | | | glued) | |
| 63 | KINH_NEUCR | KINH | Kinesin heavy chain | 158.4722 |
| 64 | STU1_ASPFU | STU1 | Protein stu1 | 26.84692 |
| 65 | NDE1_ASPFU | NDE1 | Nuclear distribution protein nudE homolog 1 | 127.4667 |
| 66 | CDC48_EMENI | CDC48 | Cell division control protein 48 | 73.2974 |
| 67 | SC61A_NEUCR | SC61A | Protein transport protein SEC61 subunit alpha | 401.4802 |
| 68 | CALX_ASPFU | CALX | Calnexin homolog | 302.1067 |
| 69 | CCG8_NEUCR | CCG8 | Clock-controlled protein 8 | 150.9155 |
| 70 | PBN1_EMENI | PBN1 | Protein pbn1 | 56.85685 |
| 71 | BIP_NEUCR | BIP | Endoplasmic reticulum chaperone BiP (EC 3.6.4.10) | 139.685 |
| | | | (Immunoglobulin heavy chain-binding protein | |
| | | | homolog) (BiP) | |
| 72 | OS9_ASPFU | OS9 | Protein OS-9 homolog | 28.80504 |
| 73 | SEC13_EMENI | SEC13 | Protein transport protein sec13 | 21.05407 |
| 74 | Y7484_EMENI | Y7484 | Uncharacterized protein AN7484 | 13.55143 |
| 75 | HSP60_EMENI | HSP60 | Heat shock protein 60 (60 kDa chaperonin) (Protein | 431.1528 |
| | | | Cpn60) | |
| 76 | HSP70_EMENI | HSP70 | Heat shock 70 kDa protein (HSP70) | 1664.506 |
| 77 | DHOM_EMENI | DHOM | Homoserine dehydrogenase (HDH) (EC 1.1.1.3) | 326.0737 |
| 78 | HSP7M_EMENI | HSP7M | Iron-sulfur cluster biogenesis chaperone, | 1895.465 |
| | | | mitochondrial (EC 3.6.4.10) (Heat shock 70 kDa | |
| | | | protein) | |
| 79 | CDC14_EMENI | CDC14 | Tyrosine-protein phosphatase cdcA (EC 3.1.3.48) | 71.77976 |
| 80 | ALDH_EMENI | ALDH | Aldehyde dehydrogenase (ALDDH) (ALDH) (EC | 7160.583 |
| | | | 1.2.1.3) | |
| 81 | PRX5_EMENI | PRX5 | Putative peroxiredoxin prxA (Prx) (EC 1.11.1.24) | 143.6626 |
| | | | (Thioredoxin peroxidase) (TPx) (Thioredoxin- | |
| | | | dependent peroxiredoxin) | |
| 82 | IPYR_EMENI | IPYR | Inorganic pyrophosphatase (EC 3.6.1.1) | 673.6631 |
| | | | (Pyrophosphate phospho-hydrolase) (PPase) | |
| 84 | ATP9_EMENI | ATP9 | ATP synthase subunit 9, mitochondrial (Lipid-binding | 114.0118 |
| | | | protein) | |
| 85 | HOG1_EMENI | HOG1 | Mitogen-activated protein kinase hog1 (MAP kinase | 6.189499 |
| | | | hog1) (EC 2.7.11.24) | |
| 86 | MIC19_EMENI | MIC19 | MICOS complex subunit mic 19 | 16.34561 |
| 87 | XKS1_ASPFU | XKS1 | Probable D-xylulose kinase A (Xylulokinase A) (EC | 609.3342 |
| | | | 2.7.1.17) | |
| 88 | ENO_EMENI | ENO | Enolase (EC 4.2.1.11) (2-phospho-D-glycerate hydro- | 161.6854 |
| | | | lyase) (2-phosphoglycerate dehydratase) | |
| 89 | G3P_EMENI | G3P | Glyceraldehyde-3-phosphate dehydrogenase | 5372.76 |
| | | | (GAPDH) (EC 1.2.1.12) | |
| 90 | NUDC_EMENI | NUDC | Nuclear movement protein nudC (Nuclear distribution | 15.81228 |
| | | | protein C) | |
| 91 | PKCA_ASPFU | PKCA | Protein kinase C (EC 2.7.11.13) | 14.25342 |
| 92 | DHE4_EMENI | DHE4 | NADP-specific glutamate dehydrogenase (NADP- | 24.43456 |
| | | | GDH) (EC 1.4.1.4) (NADP-dependent glutamate | |
| | | | dehydrogenase) | |
| 93 | TPIS_EMENI | TPIS | Triosephosphate isomerase (TIM) (EC 5.3.1.1) | 37.37789 |
| | | | (Triose-phosphate isomerase) | |
| 94 | ADH1_EMENI | ADH1 | Alcohol dehydrogenase 1 (EC 1.1.1.1) (Alcohol | 63.28495 |
| | | | dehydrogenase I) (ADH I) | |
| 95 | HSP30_EMENI | HSP30 | 30 kDa heat shock protein | 29.21878 |
| 96 | MDM10_EMENI | MDM10 | Mitochondrial distribution and morphology protein 10 | 22.91489 |
| | | | (Mitochondrial inheritance component mdm10) | |
| 97 | STCC_EMENI | STCC | Peroxidase stcC (EC 1.11.1.—) (Sterigmatocystin | 32.0421 |
| | | | biosynthesis cluster protein C) | |
| 98 | PRPC_EMENI | PRPC | 2-methylcitrate synthase, mitochondrial (Methylcitrate | 12.09284 |
| | | | synthase) (EC 2.3.3.5) ((2S,3S)-2-methylcitrate | |
| | | | synthase) (Citrate synthase 2) (EC 2.3.3.16) | |
| 100 | TMEDA_EMENI | TMEDA | Endoplasmic reticulum vesicle protein 25 | 1112.496 |
| 101 | SITA_ASPFU | SITA | Serine/threonine-protein phosphatase sitA (EC | 6.597231 |
| | | | 3.1.3.16) | |
| 102 | PP2B_EMENI | PP2B | Serine/threonine-protein phosphatase 2B catalytic | 186.2593 |
| | | | subunit (EC 3.1.3.16) (Calmodulin-dependent | |
| | | | calcineurin A subunit) | |
| 103 | ECM33_ASPFU | ECM33 | Protein ecm33 | 5325.03 |
| 104 | HAPX_ASPFU | HAPX | bZIP transcription factor hapX (Iron acquisition | 96.63311 |
| | | | regulator hapX) | |
| 105 | SIDC_ASPFU | SIDC | Nonribosomal peptide synthetase sidC (NPRS sidC) | 27.21719 |
| | | | (EC 6.3.2.—) (Siderophore peptide synthetase C) | |
| 106 | SIDA_ASPFU | SIDA | L-ornithine N(5)-monooxygenase (OMO) (EC | 74.9687 |
| | | | 1.14.13.196) (L-ornithine N(5)-oxygenase) | |
| | | | (Siderophore biosynthesis protein A) | |
| 107 | HSP90_ASPFU | HSP90 | Heat shock protein 90 (65 kDa IgE-binding protein) | 104.6264 |
| | | | (Heat shock protein hsp1) (allergen Asp f 12) | |
| 108 | PPTA_ASPFU | PPTA | 4′-phosphopantetheinyl transferase A (PPTase A) (EC | 30.57149 |
| | | | 2.7.8.7) (Acyl-carrier-protein synthase pptA) | |
| | | | (Phosphopantetheine: protein transferase pptA) | |
| 109 | CAPZB_ASPFU | CAP2 | F-actin-capping protein subunit beta | 8.92 |
| 110 | CATB_ASPOR | CATB | Catalase B | 12.48 |
| 111 | GPAA_ASPFC | GPAA | G-protein complex alpha subunit gpaA | 10.18 |
| 112 | RHOC_EMENI | RHOC | GTP-binding protein rhoC | 9.56 |
| 113 | CDC45_SCHPO | SNA41 | Cell division control protein 45 homolog | 8.01 |

### GO Terms:

BP sporulation
CC hyphal tip
BP spore-bearing structure development
BP asexual sporulation
BP reproductive fruiting body development
BP sporocarp development
BP asexual reproduction
BP regulation of asexual sporulation
BP regulation of cellular response to stress
BP hyphal growth
BP sporulation resulting in formation of a cellular spore
BP reproduction
BP regulation of conidium formation
BP sporocarp development involved in sexual reproduction
BP conidium formation
CC serine/threonine protein kinase complex
BP regulation of asexual reproduction
CC Woronin body
MF tubulin binding
BP response to endoplasmic reticulum stress
BP cellular response to osmotic stress
CC septin complex
BP positive regulation of asexual reproduction
BP TORC1 signaling
BP regulation of sporulation
BP positive regulation of conidium formation
BP transport along microtubule
BP positive regulation of single-species biofilm formation
BP regulation of sterigmatocystin biosynthetic process
BP septin ring organization
BP response to farnesol
BP cellular response to farnesol
BP organelle transport along microtubule
BP regulation of SNARE complex assembly
CC HOPS complex
BP sterigmatocystin biosynthetic process
BP asexual sporulation resulting in formation of a cellular spore
CC septin filament array
BP sterigmatocystin metabolic process
CC COPII-coated ER to Golgi transport vesicle
CC SAGA-type complex
BP spore germination
MF dynein intermediate chain binding
BP fungal-type cell wall beta-glucan biosynthetic process
BP fungal-type cell wall organization or biogenesis
BP sporocarp development involved in asexual reproduction
BP siderophore biosynthetic process
BP regulation of filamentous growth of a population of unicellular organisms
BP toxin biosynthetic process
CC fungal biofilm matrix
BP positive regulation of reproductive process
BP regulation of filamentous growth

TABLE 6
Common to both 85 and 100 RH, upregulated at both 85% and 100% compared to 50%:

| # | Gene | Uniprot protein |
|---|---|---|
| 1 | ALP2 | Alkaline protease 2 (ALP2) (EC 3.4.21.63) (Autophagic serine protease alp2) |
| | | (allergen Asp f 18) |
| 2 | CRZA | C2H2 finger domain transcription factor crzA |
| 3 | FLBA | Developmental regulator flbA |
| 4 | UBC2 | Ubiquitin-conjugating enzyme E2 2 (EC 2.3.2.23) (E2 ubiquitin-conjugating enzyme |
| | | 2) (UV sensitivity protein J) (Ubiquitin carrier protein ubc2) (Ubiquitin-protein |
| | | ligase ubc2) |
| 5 | CHSD | Chitin synthase D (EC 2.4.1.16) (Chitin-UDP acetyl-glucosaminyl transferase D) |
| | | (Class-VI chitin synthase D) |
| 6 | KAPR | CAMP-dependent protein kinase regulatory subunit (PKA regulatory subunit) |
| 7 | GPA1 | Guanine nucleotide-binding protein subunit alpha |
| 8 | APSA | Anucleate primary sterigmata protein A |
| 9 | DYL1 | Dynein light chain, cytoplasmic (8 kDa cytoplasmic dynein light chain) |
| 10 | RCO1 | Transcriptional repressor rco-1 |
| 11 | PP1 | Serine/threonine-protein phosphatase PP1 (EC 3.1.3.16) (Phosphoprotein |
| | | phosphatase 1) |
| 12 | GRRA | SCF E3 ubiquitin ligase complex F-box protein grrA (F-box and leucine-rich repeat |
| | | protein grrA) (F-box/LRR-repeat protein grrA) (SCF substrate adapter protein grrA) |
| 13 | CAN1C | Cullin-associated NEDD8-dissociated protein 1, C-terminal part (Cullin-associated |
| | | and neddylation-dissociated protein 1, C-terminal part) |
| 14 | CAN1N | Cullin-associated NEDD8-dissociated protein 1, N-terminal part (Cullin-associated |
| | | and neddylation-dissociated protein 1, N-terminal part) |
| 15 | TCSA | Two-component system protein A (EC 2.7.13.3) |
| 16 | STE20 | Serine/threonine-protein kinase ste20 (EC 2.7.11.1) |
| 17 | HSP70 | Heat shock 70 kDa protein (HSP70) |
| 18 | ENO | Enolase (EC 4.2.1.11) (2-phospho-D-glycerate hydro-lyase) (2-phosphoglycerate |
| | | dehydratase) |
| 19 | TPIS | Triosephosphate isomerase (TIM) (EC 5.3.1.1) (Triose-phosphate isomerase) |
| 20 | HSP30 | 30 kDa heat shock protein |
| 21 | MDM10 | Mitochondrial distribution and morphology protein 10 (Mitochondrial inheritance |
| | | component mdm 10) |
| 22 | ECM33 | Protein ecm33 |
| 23 | HSP90 | Heat shock protein 90 (65 kDa IgE-binding protein) (Heat shock protein hsp1) |
| | | (allergen Asp f 12) |

### Example 7

Moving beyond species: Fungal function in house dust provides novel targets for indicators of mold growth in homes

### INTRODUCTION

Increased risk of asthma and other respiratory disease are associated with exposures to microbial communities growing in damp and moldy indoor environments. The exact causal mechanisms remain unknown, and occupant health effects have not been consistently associated with any species-based measurement methods. New methods are needed to quantitatively identify homes with potentially harmful fungal growth that are not dependent upon species.

Microbial communities that grow in response to damp conditions express genes and have specific metabolic pathways and functional changes that may be strongly associated with negative health outcomes. Analysis of gene expression and metabolic changes in microbial communities have repeatedly acted as early and sensitive predictors of environmental shifts in other systems. Changing environmental factors like temperatures and moisture result in fungal growth with increased production of volatile organic compound emissions (VOCs) and mycotoxins. Damp conditions lead to increased fungal allergen potency and metabolic activity that can result in degradation of chemicals such as phthalate esters in the dust. Growing fungal communities in house dust at elevated moisture conditions results in increased expression of secondary metabolite, allergenic and pathogenic genes. Fungal growth results in increased allergen release and is also associated with proteins like hydrophobins and proteases that have direct impacts on evading host immune system response during exposure and correlate to asthma severity. Analyzing gene expression in the fungal communities in dust may yield promising options to help identify the best associations between potential microbial indicators of damp indoor environments and health effects.

The goal of this study was to identify genes consistently associated with fungal growth and associated function under damp conditions for use as potential indicators of mold in homes regardless of fungal species present. These genes may be used in the future to inform the design of better indicators of moisture damage in homes that may be associated with human health effects. A de novo metatranscriptome assembly was performed on dust collected from different homes across the US and incubated them at 3 different (50%, 85%, or 100%) equilibrium relative humidity (ERH) levels simulated in laboratory chambers. Processes associated with fungal growth enriched at elevated moisture conditions were targeted and discovered upregulated fungal genes from these growth pathways. The final set of genes are potential targets to use in homes to indicate harmful fungal growth. Such genes and their products, after further validation, can be used as diagnostic indicators of moisture damage in homes. The results of this work, through the use of novel tools, identify microbial targets of moisture signature in homes and can provide a novel perspective to further the understanding of the health implications of dampness exposures.

### Results

Gene expression was a function of moisture (adonis2 p<0.001), with fungal metabolic activity increasing with increase in moisture condition (Kruskal-Wallis p=0.003). Genes associated with fungal growth such as sporulation (n=264), hyphal growth (n=62) and secondary metabolism (n=105) were significantly upregulated at elevated ERH conditions when compared to the low 50% ERH (FDR-adjusted p≤0.001, log 2FC≥2), indicating that fungal function is influenced by growth in damp conditions. A total of 67 genes were identified as consistently associated with the elevated 85% or 100% ERH conditions and included fungal developmental regulators and secondary metabolite genes such as brlA (log 2FC=7.39, upregulated at 100% compared to 85%) and stcC (log 2FC=8.78, upregulated at 85% compared to 50%).

### CONCLUSION

These results demonstrate that gene expression of indoor fungal communities is more consistently influenced by moisture condition than species presence. Identifying genes indicative of microbial growth under damp conditions will help develop robust monitoring techniques for indoor microbial exposures and understand how dampness and mold is linked to disease.

### Methods

### 1. Participant Recruitment and Dust Collection:

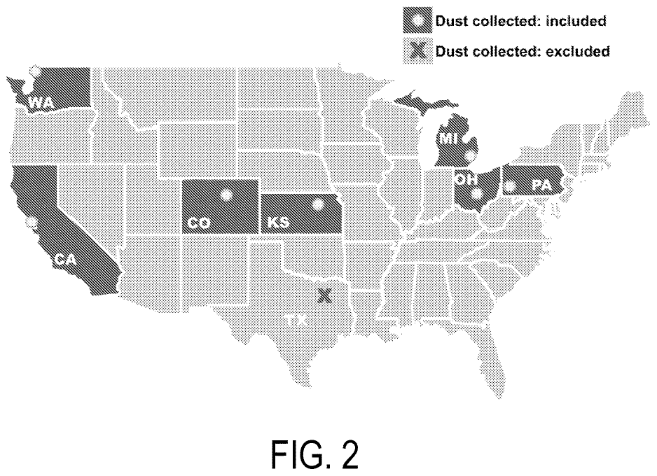

Floor dust samples were collected from nine different non-moisture damaged homes across the US from May 2021 to November 2021. Overall methods are shown in FIG. 1. Three homes were from Ohio and the remaining six were homes from six different states in the US (Table 8, FIG. 2). Due to COVID-19 restrictions, an online approach was used for participant recruitment and instructions for dust collection by participant. Using participant collected dust as a surrogate for collection by a project staff has shown to be equally effective for studies reporting allergen concentration in dust. Participants were initially recruited via social media and additional recruitment and screening were completed over email.

A Qualtrics survey (Qualtrics, Provo, UT) containing the consent form, as well as questions on relevant home and indoor environmental measures were used for screening participants. Participants were asked if there was any evidence of present water damage, moisture, leaks (such as damp carpet or leaky plumbing) or visible mold inside their homes. If participants answered in the affirmative, then these homes were not recruited for the study. The survey also contained information about the floor area and flooring type that was vacuumed, the frequency of vacuuming, types of floor cleaning, the number of occupants (adults and children), number of pets (dogs, cats, birds, and other furry pets) as well as any prior history of moisture damage and mold in participants' homes within the last five years.

One home located in Texas was initially recruited but due to consistently low quality of the extracted RNA, the dust was not included in sequencing and was excluded from this study. Two of the homes had potential moisture damage even though the participants answered in the negative to “Is there evidence of water damage, moisture, or leaks (such as damp carpet or leaky plumbing)?” (Table 8). One home (Dust sample ID: KS, Table 8) reported to have a temporary leak that occurred after heavy rains and was gone within 24 hours and the other reported to have a leak more than 10 years ago (Dust sample ID: WA, Table 8). These two samples were not excluded because they did not meet the extent of moisture damage necessary for exclusion criteria due to the extent of the damage and length of time since the damage, respectively.

Dust collection instructions were sent to the participants over email. Participants were asked to collect floor dust (>25 g), emphasizing collection from the main living areas inside their homes (living room and bedroom) using their home vacuum. If the home vacuum did not contain a vacuum bag, participants were asked to remove dust from the canister and place it in a zip top bag. Participants were then asked to ship their collected dust to the lab or have it dropped off to a designated location for pick up. Once the dust was received, all dust was screened to eliminate the presence of SARS COV-2, using a previously described protocol and no dust samples were excluded. Recruitment and dust collection procedures were approved by the Ohio State University Behavioral Institutional Review Board under study number 019B0457 for the duration of the study.

The collected dust was then hand-sieved to 300 μm to remove larger sized dust particles and was stored at 25° C. prior to chamber experiments. Dust was never frozen to maintain intact microbial communities.

### 2. Chamber Experiments

For the chamber experiments, 100 mg of sieved dust were incubated in glass chambers at 25° C. for a period of one week, at relative humidities of 50%, 85% and 100% ERH. A total of 27 dust samples being incubated (9 sites×3 conditions). Relative humidity levels in the glass chambers were maintained using salt solutions or distilled water, as detailed in previous work. 50% and 85% ERH were maintained by using salt solutions with water activities of 0.5 aw and 0.85 aw, respectively, and distilled water was used to maintain an ERH of 100%. The water activities of these salt solutions were tested for accuracy using an AquaLab™ Dew Point Water Activity Meter (Decagon 125 Devices) with a margin of error of +/−0.005.

### 3. RNA Extractions and Nucleic Acid Sequencing

Immediately following the one-week incubation, RNA was extracted from incubated dust using a previously used modified protocol of the Qiagen RNeasy PowerMicrobiome extraction kit (Qiagen, Hilden, Germany). To prevent RNA degradation from RNases, the manufacturer's protocol was modified to use 10× the concentration of β-mercaptoethanol in the first step and 70% ethanol in place of PM4 in the RNA binding step. Extracted RNA was immediately frozen at −80° C. prior to use and transported on dry ice.

To ensure high RNA quality and integrity, all RNA extracts were analyzed using the High Sensitivity RNA ScreenTape analysis on the Agilent 4200 TapeStation Bioanalyzer (Agilent, Santa Clara, CA, USA) at The Genomics Shared Resource Center (The Ohio State University Comprehensive Cancer Center Shared Resources, Columbus, OH, USA).

RNA extracts were then sent to the Yale Center for Genomic Analysis (Yale University, New Haven, CT, USA) where they were reverse transcribed and then sequenced on a NovaSeq 2×100 lane with 25 million reads per sample. RNASeq library preparation was performed using the NEBNext Single Cell/Low Input RNA Library Prep Kit (New England Biolabs, USA) and the NEB Ultra II FS (New England Biolabs, USA) workflow for Illumina. The polyA selection protocol was used to select eukaryotic mRNA. Sequence data was submitted to GenBank under accession number PRJNA1072816.

### 4. Initial Processing, Metatranscriptome Assembly and Transcript Quantification

Processing of sequenced reads followed protocols previously described. FastQC was used for quality assessment of sequences. rCorrector was utilized to correct erroneous k-mers created due to sequencing errors. After correction, reads deemed unfixable by rCorrector were filtered out using the TranscriptomeAssemblyTools package.

De novo metatranscriptome assembly was conducted using Trinity with default settings and was run on the Ohio Supercomputer (Ohio Supercomputer Center, Ohio). Trimmomatic within the Trinity pipeline was used to remove poor quality reads and contigs with a length less than 300 base pairs (bp). Contigs from the Trinity assembly were clustered using CD-HIT-EST based on 80% sequence similarity. These clusters from CD-HIT-EST represent all expressed contigs and constitutes the full transcriptome.

Abundance estimation and alignment were run within the Trinity pipeline with default parameters. RSEM was used to estimate transcript abundance in each sample and to determine transcript-level expression counts of the RNA-Seq fragments for each transcript using alignment-based quantification. Bowtie2 was used to align the quality trimmed paired-end reads after Trimmomatic to the full transcriptome created using CD-HIT-EST. Read coverage was then quantified using Samtools to capture read alignment statistics for concordant read pairs (yielding concordant alignments 1 or more times to the CD-HIT-EST transcriptome) with a MAPQ greater than 2.

Transcript-level abundance estimates were used to construct a matrix of counts and a matrix of normalized expression values. Normalized expression values include Counts Per Million (CPM), Transcripts per Million (TPM) and Trimmed Mean of M-values (TMM) and account for transcript length, number of reads mapped to a transcript, total number of reads over all transcripts and library size (sequencing depth). Gene-level count and gene-level normalized expression matrices were calculated using txImport implemented directly in the Trinity pipeline.

### 5. Differential Expression Analysis

DESeq2 was used within the Trinity pipeline to perform Differential Gene Expression (DGE) analysis of expressed genes. DGE performed using gene-level counts were used for downstream target gene identification. Performing differential expression analysis on gene levels, in addition to contig or transcript levels, improves interpretation of annotated contigs and potentially increases statistical power. Pairwise comparisons between the three ERH conditions (50%, 85% and 100%) were performed, giving rise to six pairwise ERH comparisons. Genes that were most differentially expressed based on the most significant False Discovery rate (FDR) and log 2FC (log 2 fold change) values were extracted and used for subsequent Gene Ontology (GO) enrichment analysis.

### 6. Functional Annotation and Gene Ontology Enrichment

Transcripts were annotated using Trinotate, designed for comprehensive functional annotation of de novo transcriptomes. Trinotate integrates all functional annotation data into an SQLite database, which is used to create a whole annotation report for the transcriptome. For functional annotation, Trinotate used BLAST+ sequence homology search of transcripts and predicted coding regions against the SwissProt database and protein domain identification using a HMMER search against the PFAM database. Predicted coding regions were identified using TransDecoder that utilizes a minimum length Open Reading Frame (ORF) found in a transcript sequence. The TrEMBL/SwissProt database was used for Gene Ontology (GO) and KEGG assignments of transcripts using Trinotate. KEGG assignments for genes were analyzed using the KEGG Mapper tool to identify the number of metabolic pathways and visualized using the iPath3 tool as metabolic pathway maps.

GOseq, developed specifically to account for gene length bias in RNA-seq data, was used within the Trinity pipeline to perform functional GO enrichment testing. Results from the GO enrichment was analyzed for enriched GO categories based on significance of enrichment using FDR values and the number of DE genes within these GO categories at each pairwise ERH comparison.

7. Identifying Potential Target Genes Associated with Fungal Growth at High Moisture

To identify fungal genes that are strongly upregulated at higher moisture conditions, GO enrichment was performed on the most highly significant and differentially expressed genes with a cutoff of FDR-adjusted p≤0.001 and log 2FC≥2. GO enrichment results were then analyzed for GO terms associated with fungal growth that were significantly enriched at higher moisture conditions (FDR<0.05). Higher moisture conditions comprised of GO terms enriched at 100% compared to 85% ERH, enriched at 100% compared to 50% ERH and enriched at 85% compared to 50% ERH. Finally, genes upregulated within these GO categories associated with fungal growth at higher ERH and having a known fungal annotation (BLASTX) were used to identify genes as potentially targets that are indicative of mold growth.

Target genes were chosen based on the criteria that (i) genes have a log 2FC≥5 (ii) genes are expressed in at least two-thirds of sampling sites (n≥6, out of a total n=9 locations) and (iii) genes are not expressed at the 50% ERH condition in any sample. At least 80% of the all identified target genes were ensured to follow the criteria. Exceptions were only made in criteria (ii) if the gene was essential for fungal growth, where genes upregulated in at least three sampling sites were included. Counts in the 0-10 range are usually considered ‘noise’ and therefore, the target genes were required to have a count <10 in every site at 50% ERH. Gene expression heatmaps were plotted based on TMM-normalized expression values for direct comparison of gene expression across samples. Log2 transformed and mean-centered standardization were performed prior to analysis to reduce bias towards highly expressed transcripts.

### 8. Species Identification in Samples

Similar to incubations for RNA extractions, 50 mg of dust was incubated for 1-week at 50%, 85% and 100% ERH and was used for the DNA extractions. DNA extractions were performed using the Maxwell RSC PureFood GMO and Authentication Kit (Promega, USA) using the protocol for lysing food and seed samples. Modifications included alterations to the bead beating in which 0.3 g of 100 μm glass beads, 0.1 g of 500 μm glass bead, and 1 g of PowerBeads (Qiagen, USA) were used for the bead mix and bead beat for 5 minutes. In addition, the incubation step was modified to allow the samples to be incubated for 30 minutes at room temperature. This was followed by centrifuging for 10 min at 13,000 rpm in combination with lysis buffer, with the final elution volume being modified to 75 μL. DNA extracts were stored at −20° C. and transported on dry ice. DNA extracts were processed for qPCR and amplicon sequencing following the protocols described previously. Sequence data was submitted to the GenBank under accession number PRJNA1072816.

A DADA2-based bioinformatics pipeline customized for ITS sequences was run using R on Ohio Supercomputer (Ohio Supercomputer Center, Ohio). Adapters were first removed using Cutadapt, BioStrings, and ShortRead. Denoising was performed using DADA2 where the maxEE and truncQ parameters of the filterAndTrim function were both set to eight following Rolling et al. The UNITE version 9.0 database was used for taxonomic identification.

### 9. Statistical Analysis

The statistical analysis software R (v.4.2.2) was used to perform statistical testing. To compare gene expression profiles based on moisture condition, relationships between biological replicates were compared across samples using Principal Component Analysis (PCA), which is identical to Principal Coordinates Analysis (PCoA) using Euclidean distances. Gene expression values in Counts Per Million (CPM) that account for library size normalization were used for PCA. Log2 transformed and mean-centered standardization, typically applied in gene expression studies, were performed prior to analysis to reduce bias towards highly expressed transcripts. PCoA was performed for relative abundance of fungal species using Bray-Curtis distances. The adonis2 function in R using the vegan package was used to determine statistical significance of ERH groupings (p<0.05) from the Euclidean and Bray-Curtis distance matrix. The test employed 10,000 permutations and used FDR to adjust for multiple comparisons. Significance was defined at FDR-adjusted p<0.05. A 95% confidence ellipse using the stat_ellipse function within the ggplot2 package was created to compare moisture conditions to each other, where a smaller ellipse around the data indicates less variance in that dataset group.

The Spearman rank correlation coefficient was calculated using the corrplot package for differentially expressed genes based on moisture condition. Only the correlation coefficients that were significant (p<0.05) were considered. The Spearman rank correlation coefficient determines the strength and direction in the relationship between the data where a value of 1 indicates the strongest positive correlation.

To identify species with differences in abundance between the ERH levels, the Kruskal-Wallis test was first performed to determine significant difference (p<0.05), followed by pairwise Wilcoxon rank sum test using FDR to control for multiple comparisons. To determine significant differences between the number of fungal genes present by ERH condition, Kruskal-Wallis test followed by pairwise Wilcoxon rank sum test was performed, with FDR to adjust for multiple comparisons. These tests were also used for determining differences in absolute fungal concentrations based on ERH condition. Kruskal-Wallis tests were used as a non-parametric alternative to ANOVA when the data was determined to not be normally distributed (Shapiro-Wilk p<0.05).

### 10. Visualization

Figures in the manuscript were generated using R scripts within Trinity, ggplot2, ComplexHeatmap, corrplot, iPath3, Canva, Adobe Illustrator [v.28.3] and Inkscape [v.1.3].

### Results

### Overview of Metatranscriptomic Dataset:

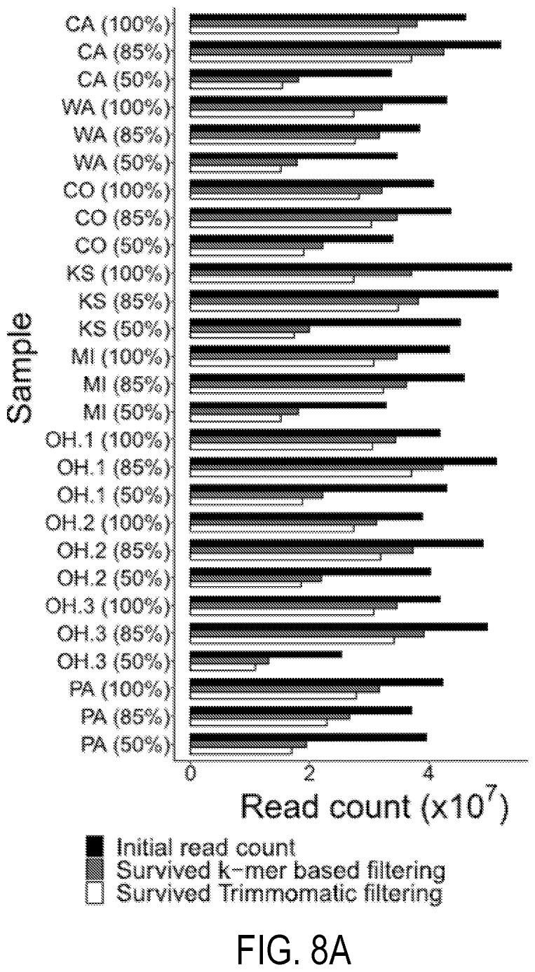

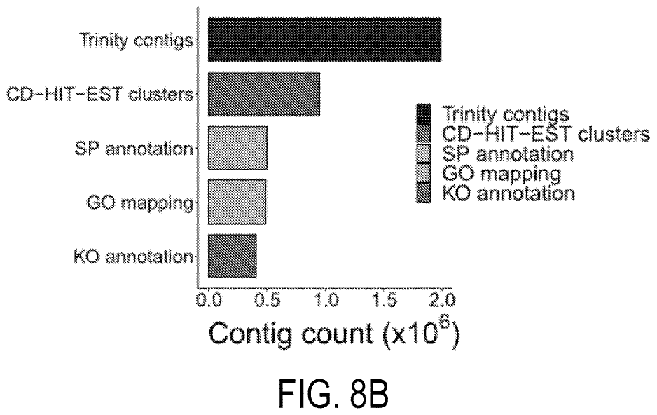

Sequencing produced a total of 700,682,204 paired-end reads. Trinity assembled all high-quality reads into 1,983,474 contigs and 1,023,948 genes. The median contig length was 556 base pairs (bp) with a minimum size threshold of 300 bp. After quality filtering, on average 70.07% of reads mapped back to the full transcriptome and a total of 54.83% of quality-filtered reads were mapped (more quality statistics in FIG. 8). The percent of reads that survived the quality filtering and mapping is similar to other metatranscriptomic studies, including studies performed using house dust.

Moisture is More Consistently Associated with Microbial Function than Species Presence:

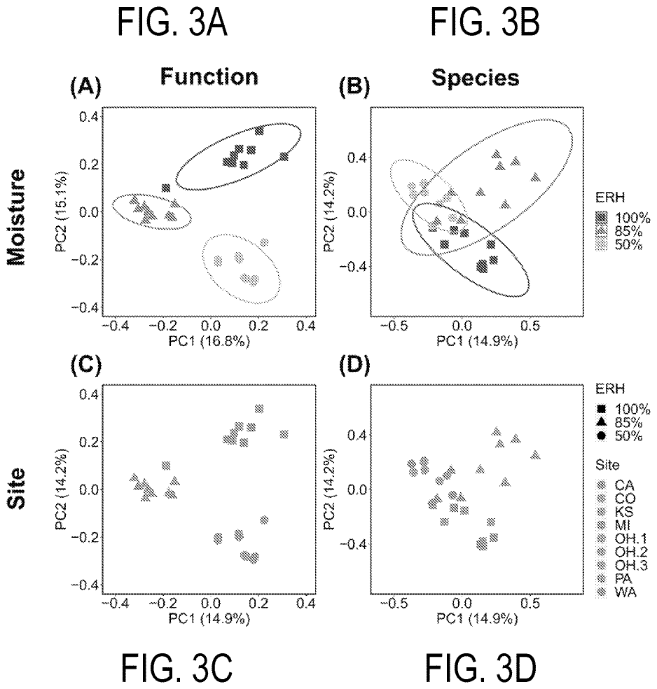

Relative humidity condition is significantly associated with both gene expression (FIG. 3A) and species (FIG. 3B) in the samples (adonis2 R²=0.28, p≤0.0001 and adonis2 R²=0.21, p≤0.0001, Table 9). The difference is more pronounced in gene expression with non-overlapping ellipses compared to species with overlapping ellipses, indicating that this may be a stronger predictor of moisture in a sample than species. When looking at species (FIG. 3D), some samples cluster together more strongly by site than by ERH condition (CA 50%, CA 85% and WA 50%, WA 85%), which cannot be observed for gene expression (FIG. 3C).

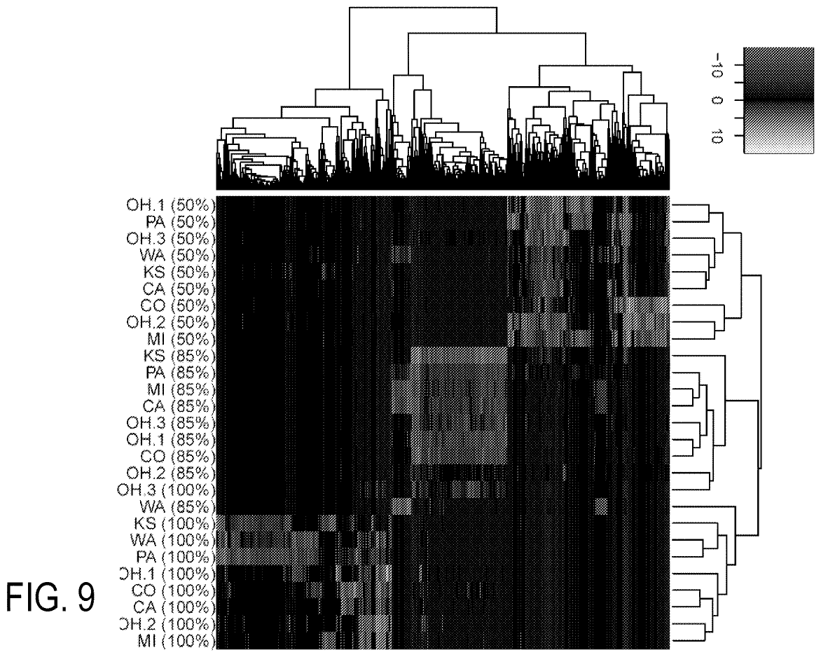

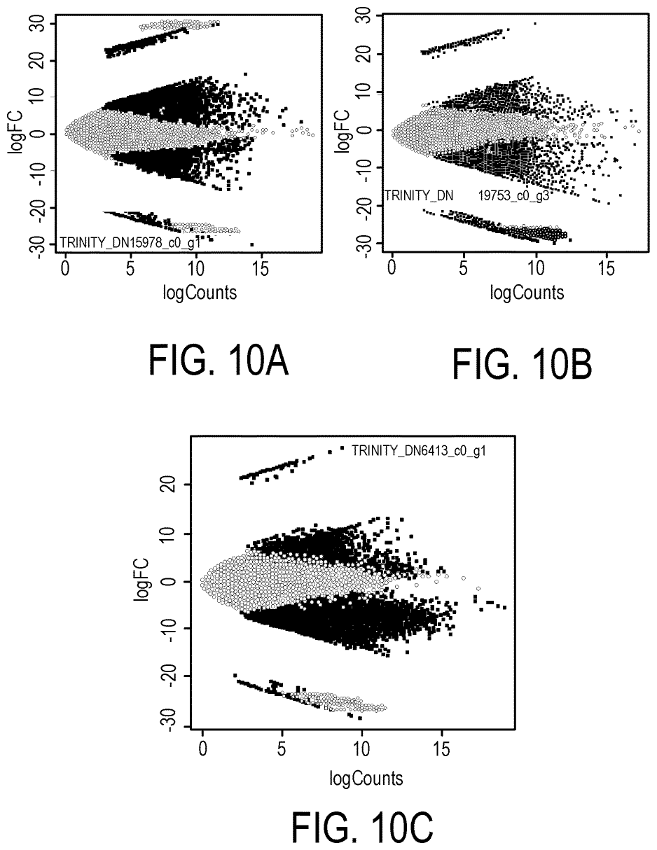

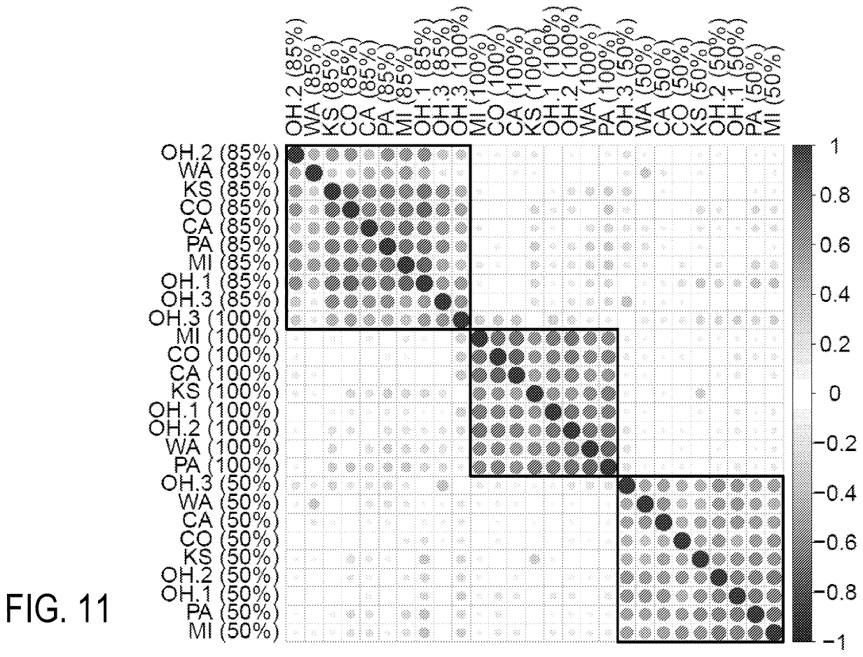

Samples cluster by moisture condition based on the gene expression heatmap (FIG. 9) indicating gene expression is a function of relative humidity condition. These differences can also be seen in the MA plots (FIG. 10) which indicate differentially expressed genes at each pairwise RH comparison, 100% vs 85%, 100% vs 50% and 85% vs 50%. Differentially expressed genes were highly correlated within moisture conditions (Spearman correlation, rho>0.5, p<0.5), and samples grouped by RH condition based on hierarchical clustering (FIG. 11).

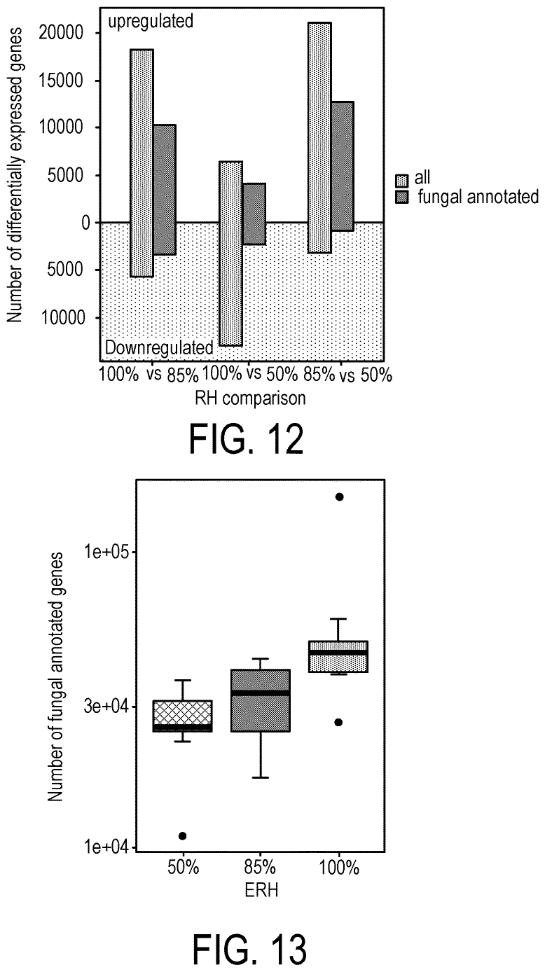

Many fungal genes are consistently expressed only at elevated moisture conditions (FIG. 12, Table 7). Thousands of genes were upregulated at elevated ERH conditions (100% or 85% compared to 50% ERH) in a majority of samples and were not expressed at the low 50% condition (Table 7). 732 genes were upregulated in all sites at either 100% or 85% ERH conditions when compared to 50% and were not expressed in any 50% samples (Table 7). Overall, this indicates that many genes are expressed only at 100% or 85% ERH or both. In contrast, no fungal species was found to be consistently associated with elevated ERH in all samples. Species that were more abundant at elevated ERH conditions (100% or 85%) were also present at the low 50% condition, similar to previous studies. Out of the two species that were more abundant at the 85% ERH condition (Aspergillus ruber and Aspergillus intermedius), both were found at 50% ERH (Table 10). For instance, Aspergillus ruber that is more differentially abundant at 85% ERH (in all sites), was also found in 7 sites at 50% ERH. Only one species (Chaetomium angustispirale) that was more abundant at 100% ERH in 8 sites (compared to 50%), was not found in any of the 50% ERH samples (Table 10). Overall, these results suggest that utilizing genes associated with elevated ERH conditions may potentially be able to overcome the inconsistencies associated with using species as indicators of moisture.

TABLE 7
Number of upregulated fungal genes and fungal species that are found to be more
abundant at 100% compared to 50% and 85% compared to 50% (FDR-adjusted p < 0.05).
Gene Expression

| | Upregulated at 100% | Upregulated at 85% vs |
|---|---|---|
| | vs 50% | 50% |
| Number of genes upregulated (FDR-adjusted | 4141 | 12845 |
| p < 0.05) | | |
| Number of genes upregulated in at least 6/9 | 3188 | 5437 |
| sites and not expressed at 50% | | |
| Number of genes upregulated in at least 8/9 | 2030 | 2528 |
| sites and not expressed at 50% | | |
| Number of genes upregulated in all sites and | 324 | 431 |
| not expressed at 50% | | |

Species

| | More abundant at | More abundant at 85% |
|---|---|---|
| | 100% vs 50% | vs 50% |
| Number of species differentially abundant | 3 | 2 |
| (FDR-adjusted p < 0.05) | | |
| Number of species more abundant in at least | 1 | 0 |
| 6/9 sites and not found at 50% | | |
| Number of species more abundant in at least | 1 | 0 |
| 8/9 sites and not found at 50% | | |
| Number of species more abundant in all sites | 0 | 0 |
| and not found at 50% | | |

Fungal Metabolic Activity Increases with Increase in Moisture:

Fungal gene expression (based on the number of fungal annotated genes) increased with increase in relative humidity condition (Kruskal-Wallis p=0.003), with the 100% condition, on an average, having 2.1 times the number of fungal annotated genes present at 50% ERH (Wilcoxon p=0.002, FIG. 13, Table 11).

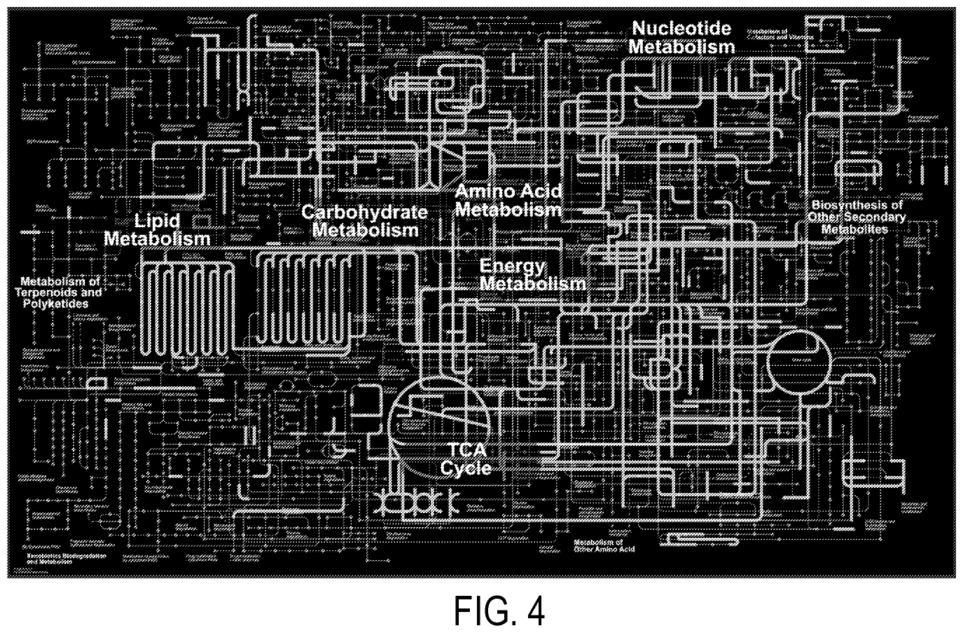

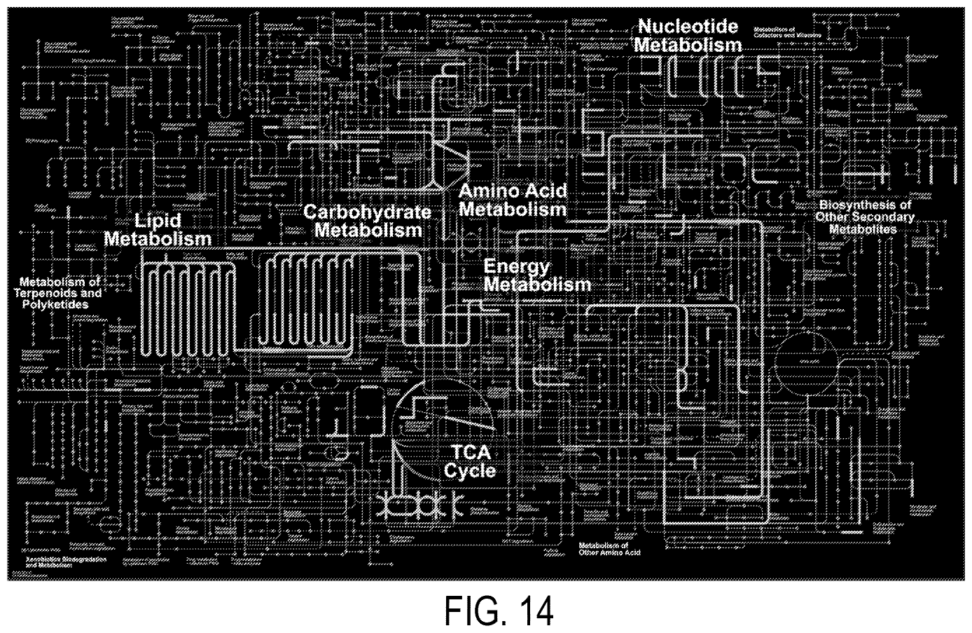

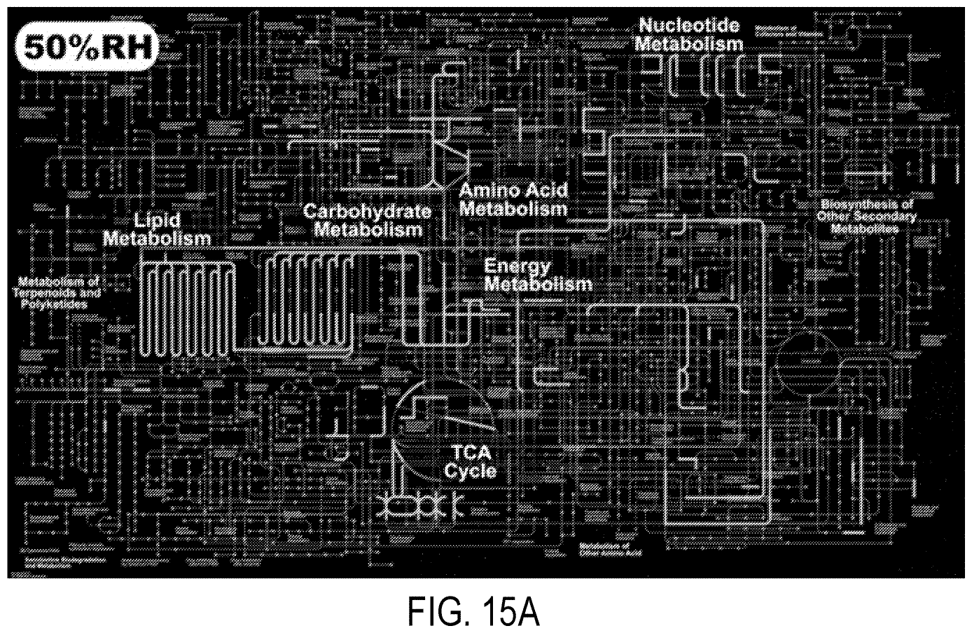

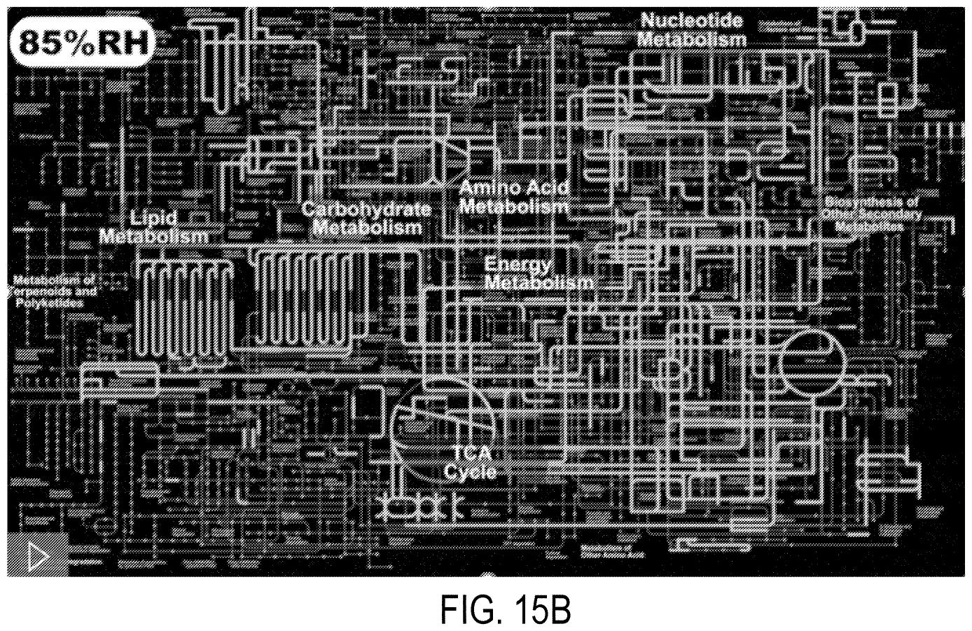

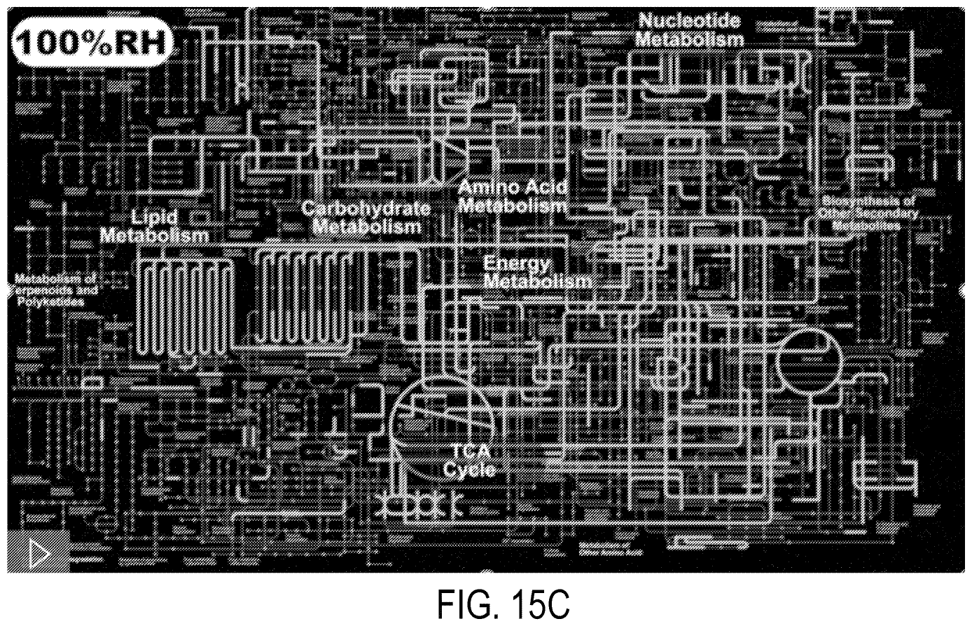

There were a greater number of upregulated fungal genes at the 100% or 85% ERH conditions compared to the lower 50% ERH (p≤0.001, log 2FC≥2, FIG. 12, Table 12). There were 1.8 times the number of significantly upregulated vs downregulated genes at 100% ERH when compared to 50% and 3.2 times the number of significantly upregulated genes at 100% when compared to the 85% ERH condition. A greater number of fungal metabolic pathways were found upregulated at 100% and 85% ERH conditions than at 50% (FIG. 4, FIGS. 14 and 15). 100% ERH (n=383) had 3.3 times the number of fungal metabolic pathways as 50% ERH (n=117), based on the 100% versus 50% ERH comparison (Table 13).

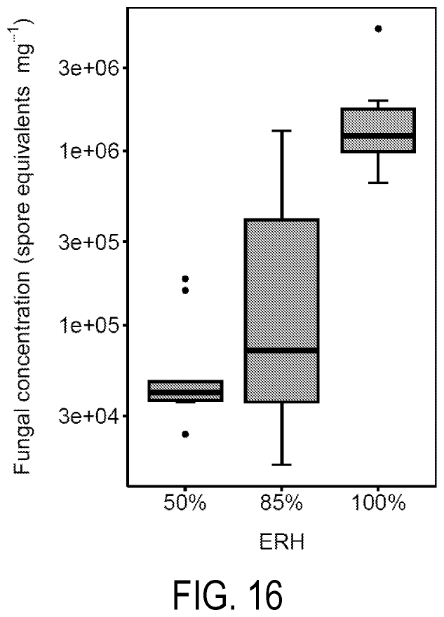

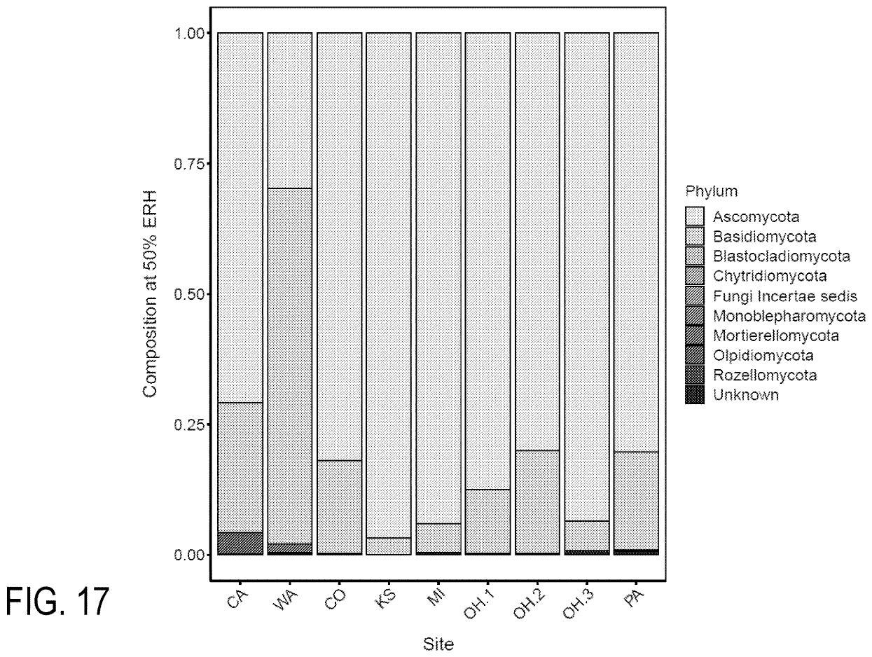

Similar to previous studies, fungal concentration increased with increase in ERH condition (Kruskal-Wallis p=0.007, FIG. 16). The fungal taxa present in the dust at the initial 50% ERH condition varied by site, with the majority in most sites being Ascomycota (FIG. 17).

Genes Associated with Fungal Growth are Upregulated at High Relative Humidity Conditions:

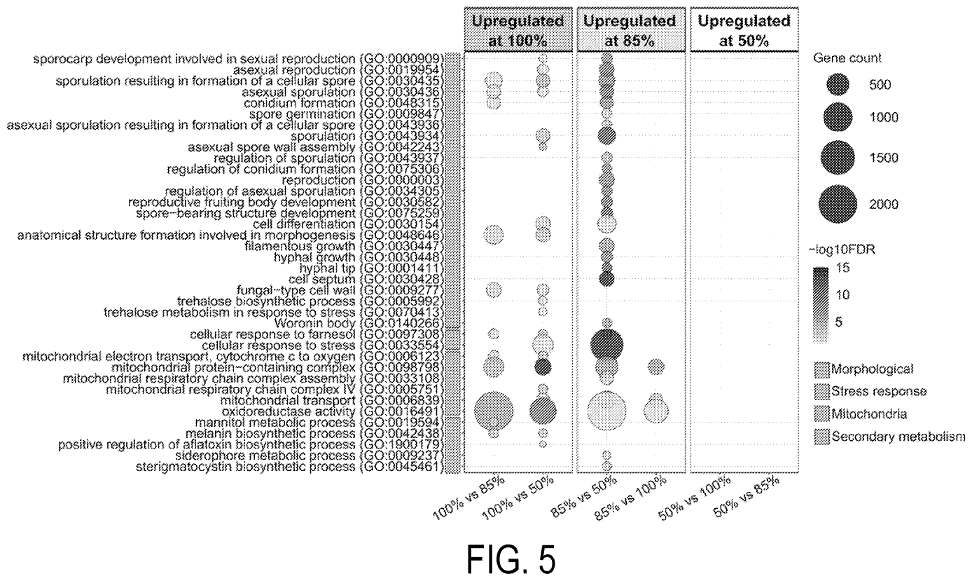

GO terms associated with fungal growth are enriched at the 100% and 85% ERH conditions compared to 50% ERH (FDR<0.05) (FIG. 5). No growth associated GO terms (n=0, FDR<0.05) were enriched at low ERH condition (50% ERH as the upregulated condition), indicating that overall, gene expression associated with fungal growth is associated with higher moisture conditions (Table 14 and 15).

Morphological processes that occur during fungal growth are significantly enriched at both the 100% and 85% ERH conditions when compared to the low 50% ERH condition. Filamentous fungi begin to grow by elongating the tip of their hyphae, which is followed by the formation of reproductive growth structures and the production of spores (sporulation). Genes associated with the GO term “sporulation” were upregulated at the 100% and 85% ERH conditions when compared to 50% ERH. GO terms associated with hyphal elongation such as “cell septum” and “hyphal tip” were significantly enriched at 85% ERH when compared to 50% (FDR<10-10, FIG. 5, Table 13). The GO term “anatomical structure formation involved in morphogenesis” had the highest number of upregulated genes (n=323) and was significantly enriched at 100% when compared to 85% ERH.

GO terms associated with fungal secondary metabolism are significantly enriched at 100% and 85% ERH conditions when compared to 50% ERH (FDR<0.05). Secondary metabolic processes are chemical reactions and pathways that are not required for the growth and maintenance of the organism. In filamentous fungi (mold), secondary metabolism includes the production of natural products such as pigments and harmful toxins such as mycotoxins and is often accompanied by fungal morphological growth and virulence. Genes belonging to the term “melanin biosynthetic process” that are associated with the production of the fungal pigment melanin were significantly upregulated at 100% in both the 100% vs 85% and 100% vs 50% ERH comparisons ((FDR=0.001 and FDR=0.0005, respectively). Genes associated with fungal mycotoxin production, belonging to GO terms such as “sterigmatocystin biosynthetic process” and “positive regulation of aflatoxin biosynthetic process,” were significantly upregulated at 100% and 85% ERH conditions when compared to 50% ERH (FDR<0.05).

Genes associated with stress response were highly upregulated at 100% and 85% when compared to the low 50% ERH condition. For many filamentous fungi, the act of growing hyphal structures likely places significant stress on the secretory system. 1364 genes belonging to the term “cellular response to stress” (FDR=2.54×10⁻²¹) were upregulated at 85% when compared to 50% ERH condition. These included the bipA and cdc48 genes that function during secretory stress responses and are also required for normal hyphal growth and morphology.

Genes associated with mitochondrial respiration and oxidoreductase activity were also found to be upregulated at 100% and 85% when compared to the low 50% ERH condition. Morphological transitions that occur during growth and virulence in fungi have been associated with mitochondrial respiratory activity in fungi. A total of 214 genes belonging to the “mitochondrial protein-containing complex” GO term were found to be significantly upregulated at the 100% condition when compared to 50% ERH.

Hydrophobins, Developmental Regulators and Secondary Metabolite Genes are Consistently Associated with Moisture:

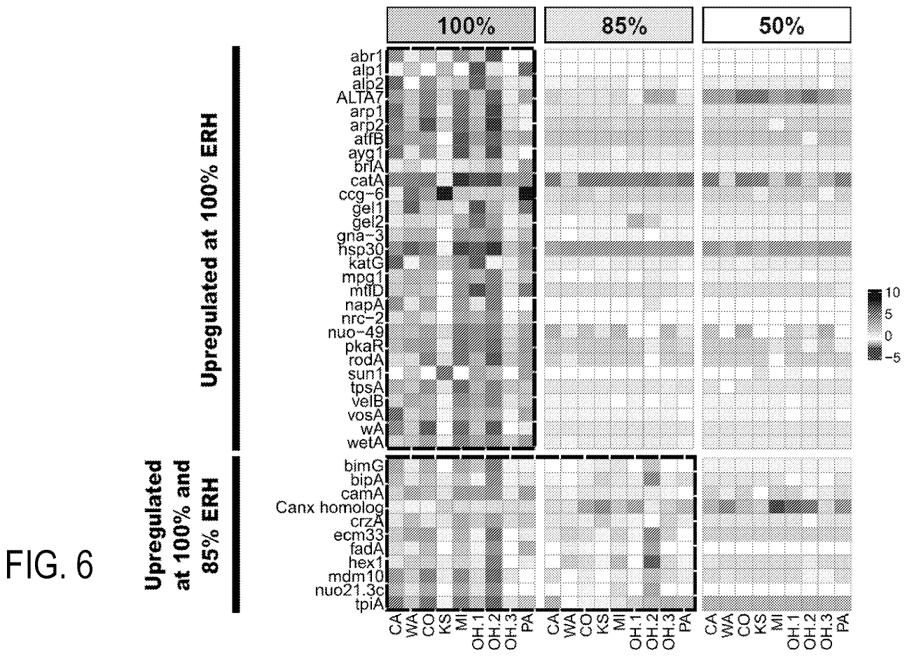

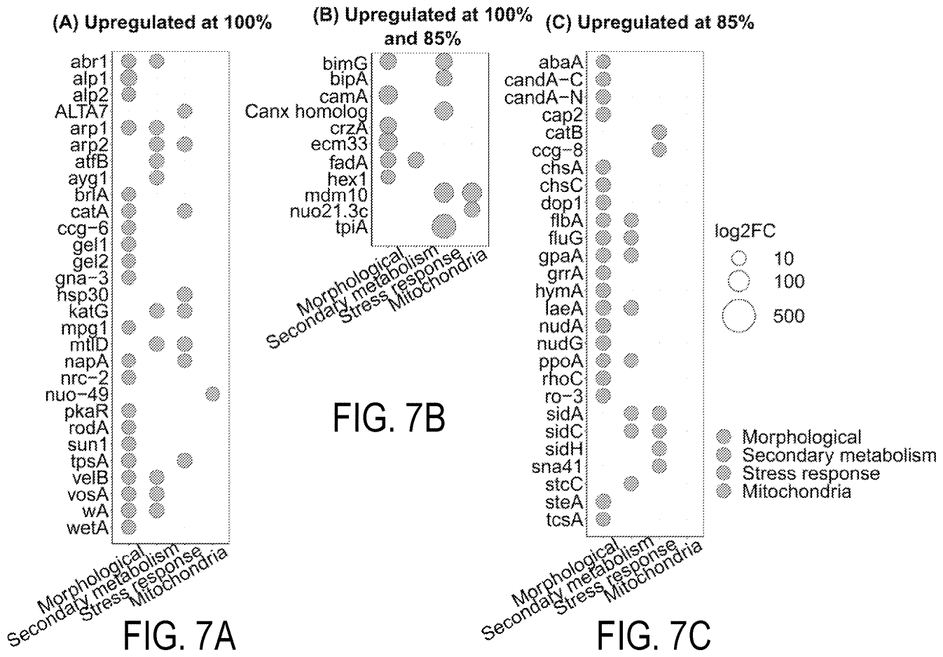

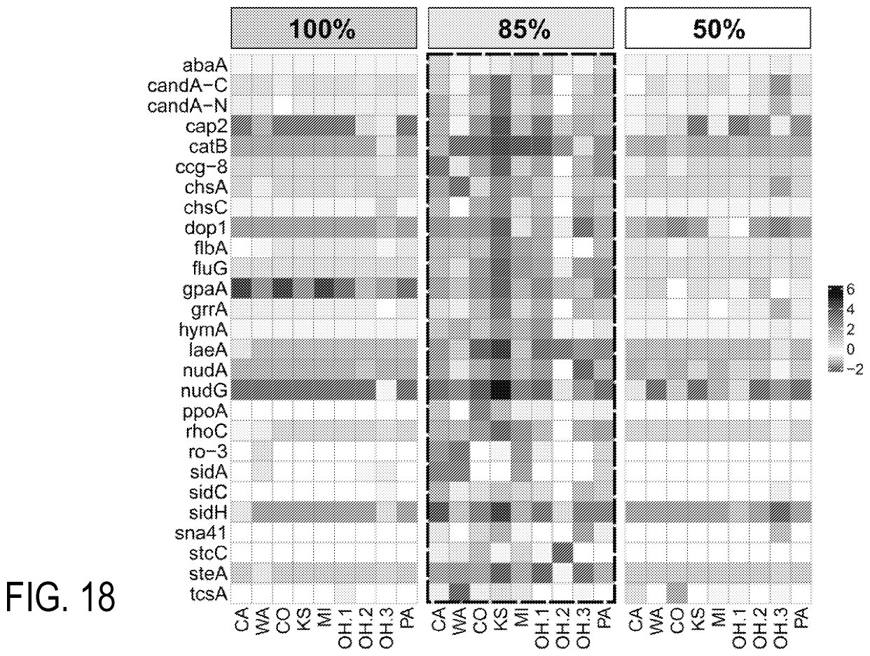

Overall, fungal growth associated genes (n=67) fell into 3 groups based on ERH condition, i.e., (1) Upregulated at 100% ERH (n=29), (2) Upregulated at both 100% and 85% ERH (n=11) and (3) Upregulated at 85% ERH (n=27) (FIG. 6, FIG. 18, Table 14). Across all groups, a majority of the genes (n=47) were associated with fungal morphological processes (FIG. 7). Genes were also associated with stress response (n=19), secondary metabolism (n=19) and mitochondria-related processes (n=3).

The most differentially expressed genes at 100% ERH were the Alkaline protease gene, alp1 with log 2FC of 25.69 (100% vs 85% ERH comparison), followed by the pigment related genes arp1 and wA (log 2FC=12.34 and 12, respectively both at 100% vs 85% ERH comparison). These genes were predominantly part of morphological growth GO terms such as “conidium formation” (GO: 0048315) and “sporulation resulting in formation of a cellular spore” (GO: 0030435) (Table 15 and 16). The arp1 and the wA gene were also associated with pigment biosynthesis, with arp1 associated with the term “melanin biosynthetic process” (GO: GO: 0042438). Other morphological growth associated genes that were highly expressed included the hydrophobin gene, rodA with log 2FC=8.68 at the 100% vs 50% ERH comparison. Developmental regulator genes such as the brlA gene were also highly expressed with log 2FC=7.39 at the 100% vs 85% comparison.

The most consistently upregulated genes at both 100% and 85% ERH included mitochondria related genes such as mdm10 showed upregulation at both 85% and 100% with log 2FC=71.87 and was associated with the mitochondrial protein-containing complex” (GO: 0098798) GO term. The highly expressed fadA gene was associated with both morphological processes and secondary metabolic processes such as (log 2FC=17.42) such as “sporulation” (GO: 0043934) and “sterigmatocystin biosynthetic process” (GO: 0045461).

At the 85% ERH, 20 out of the 27 total upregulated genes were associated with morphological growth processes. Of these, laeA and dop1 functioning as morphological growth regulators had the highest differential expression with log 2FC=11.65 and 11.59 respectively both at 85% vs 50% ERH comparison. The laeA gene additionally functions as a secondary metabolic gene and is associated with the GO term “sterigmatocystin biosynthetic process” (GO: 0045461). Similar to the 100% upregulated condition, developmental regulator genes such as flbA and fluG were highly expressed with log 2FC=8.69 and 10.76 respectively (85% vs 50% ERH comparison) having both morphological as well as secondary metabolic functions such as “sporulation” (GO: 0043934) and “sterigmatocystin biosynthetic process” (GO: 0045461).

### DISCUSSION

Species-based approaches have been unsuccessful in identifying a consistent microbial indicator of moisture damage in buildings that is more associated with health outcomes than subjective measures of visual or odor assessment. These results demonstrate that gene expression of indoor fungal communities is more strongly driven by moisture condition that species differences in microbial communities. Genes expressed during growth showed consistent upregulation at elevated moisture conditions and may be used as improved indicators of water damage. The results of this study provide important direction that will be crucial in the search for quantitative indicators of moisture and mold damage in homes.

Function, Rather than Species, is Consistently Influenced by Moisture:

Buildings contain hundreds of different fungal species that vary by geographic location, building use, occupancy, and other factors. Because these species vary greatly, the species composition also changes in different ways upon exposure to moisture. However, there are gene clusters shared across the fungal kingdom. It was hypothesized that gene expression may be more consistently and clearly associated with ERH condition than species composition, and in fact the results support that. 735 fungal annotated genes were found that were upregulated at elevated ERH conditions (either 100% or 85% ERH or both) across all 9 samples from 6 distinct geographical sites across the US. For instance, growth associated genes encoding for the hydrophobin rodA (that supports aerial growth and attachment to solid supports) and the sporulation-specific catalase catA were upregulated at 100% ERH at every single sampling site (FIG. 5) and not expressed at 50% ERH condition in any site (Table 16).

Genes associated with a specific metabolic or functional response can span across a wide range of taxa, enabling the measurement of coordinated and multispecies responses to environmental changes. Similar processes occur in other environmental systems such as marine environments, soil, and groundwater microbiomes. For instance, marine picoplankton populations exhibit cross-species, synchronous and tightly regulated patterns of gene expression for many genes, particularly those genes associated with growth and nutrient acquisition. Many microbial functions are conserved across taxa and may contribute to the higher sensitivity of gene expression to environmental changes over taxonomic composition.

Gene Expression Associated with Health Effects: Implications for Housing Quality:

The work in this study also provides advanced insights into the microbial activity that occurs in damp indoor environments that are associated with health effects. Genes associated with allergens and mycotoxins were found upregulated at elevated ERH conditions when compared to 50%, similar to prior studies. These included genes associated with fungal allergens such as Alt a 7 upregulated at 100% compared to 50% ERH and secondary metabolire genes associated with mycotoxin production (GO: 0045461) such as stcC that was upregulated at 85% when compared to 50% ERH. Genes associated with fungal growth also had associations with negative health effects in prior studies. The fungal alkaline protease gene alp1 (also known as the allergen Asp f 13 gene) was upregulated at 100% compared to 85% ERH and has strong correlations with asthma severity and respiratory dysfunction and potential functions in promoting fungal growth and infection development in the host. The hydrophobin gene associated with fungal spore surfaces, rodA (rodlet protein or rodlet layer), can evade human host immune responses. Genes related to mitochondrial functions such as mdm10, were upregulated at 85% ERH compared to 50% and have potential associations with fungal virulence by regulating stress responses and mediating morphogenetic transitions. Fungal exposure is linked to asthma exacerbations in both children and adults and these results suggest that the metabolic state, rather than specific taxa, may be driving negative health effects linked to damp buildings. That said, there is still lack a complete understanding of the response of the microbiome under damp conditions in homes and the mechanisms linking fungal exposure to health effects.

### Function can Help Identify Novel Targets to Indicate Mold Growth Indoors:

Targeting metabolic functions specific to high moisture conditions is a more robust approach than species-based indicators to identifying microbial indicators of moisture damage. Targeting genes that are upregulated at both the 100% and 85% ERH conditions (compared to 50%) or using multiple genes where some are indicative of the 100% condition and others of 85%, may be better at detecting microbial changes at the onset of dampness. A quantitative microbial indicator of moisture would, at minimum, need to be consistently upregulated in most (if not all) sampling sites at high ERH conditions, but not expressed at the low 50% condition. Such a fungal target could be used in homes similarly to fecal indicators in water systems. For instance, crAssphage is a human gut-associated bacteriophage can be used as a viral indicator of human fecal pollution and can potentially be quantitatively representative of viral pathogen fate and concentration changes in sewage-contaminated waters. The target gene groups reported in the study can measure moisture and mold damage in homes and help correlate these measurements to occupant health exposure and outcomes in a quantitative manner. Ultimately, these targets can be integrated into standards and regulations.

### CONCLUSION

Overall, this work improved understanding of the functional processes occurring within indoor fungal communities and demonstrated that high moisture is associated with growth processes, upregulation of secondary metabolic pathways, and increased mitochondrial activity. Upregulation of these genes was more strongly associated with high moisture than taxonomic measures of species. Together with other work, these findings show that there is a need to move beyond the assumption that a microbial indicator of moisture in homes must be identified through species-based approaches or that an indicator is solely taxonomic in nature. Ideally, selected target genes or their products from the gene groups after further validation can be used in quantitative measurement systems that can perform sensitive detection of moisture damage in homes. Such a system addresses the both the substantial financial and health impact of mold growth in the society and be especially important for vulnerable groups such as children with asthma.

TABLE 8
Dust collection, housing, indoor environmental and occupant health characteristics of 9 participating
homes (N = 9) in the study. Responses listed as ‘N/A’ indicate that the participant did not answer.

| | | | | | Environmental | | | |
|---|---|---|---|---|---|---|---|---|
| | Dust Collection | | | | parameters | | Housing Characteristics | |
| Dust | Location | Time | Dust | Area | | Relative | | Flooring type |
| sample | (state and | of dust | collected | sampled/ | Temperature | Humidity | Number of | that was |
| ID | geocode) | collection | from | vacuumed | (° F.) | (% RH) | occupants | vacuumed |
| OH.1 | Ohio (432) | Jun 2021 | Living/family room, | 360 | 70 | 30 | 4 | Wall to wall |
| | | | Hallway, Entry way, | | | | | carpet, |
| | | | Stairs | | | | | Area rug, Tile |
| OH.2 | Ohio (432) | May 2021 | Living/family room, | 220 | 75 | 60 | 3 | Area rug, Tile, |
| | | | Kitchen, Hallway, | | | | | Hardwood |
| | | | Entry way, Dining | | | | | |
| | | | room, Stairs | | | | | |
| OH.3 | Ohio (432) | May 2021 | Bedroom | 750 | 82 | N/A | 2 | Wall to wall |
| | | | | | | | | carpet |
| WA | Washington (982) | June 2021 | Bedroom, Living/ | 500 | 65 | 50 | 4 | Wall to wall |
| | | | family room, | | | | | carpet, |
| | | | Hallway, Dining | | | | | Tile |
| | | | room, Bathroom | | | | | |
| PA | Pennsylvania (152) | July 2021 | N/A | N/A | N/A | N/A | N/A | N/A |
| KS | Kansas (665) | November 2021 | Living/family room, | 900 | 72 | 40 | 2 | Area rug, |
| | | | Kitchen, Entry way, | | | | | Hardwood |
| | | | Dining room | | | | | |
| MI | Michigan (483) | June 2021 | Bedroom, Living/ | N/A | 74 | N/A | 4 | Wall to wall |
| | | | family room, | | | | | carpet, |
| | | | Hallway, Stairs | | | | | Area rug |
| CC | Colorado (802) | June 2021 | Living/family room, | 700 | 70 | 30 | 2 | Wall to wall |
| | | | Kitchen, Entry way, | | | | | carpet, |
| | | | Dining room | | | | | Hardwood |
| CA | California (941) | July 2021 | Living/family room | 375 | 65 | 70 | 3 | Area rug |

| | Housing Characteristics | | | | | Occupant Health |
|---|---|---|---|---|---|---|
| | How do you clear the | | Moisture and mold characteristics | | | Physician |
| Dust | floor? (Select all | | Evidence | History | | diagnosed |
| sample | that apply) - Selected | Cleaning | of water | of water | | healthy |
| ID | Choice | frequency | damage | damage | Notes | conditions |
| OH.1 | Vacuum, Sweep with a broom, | Weekly | No | No | | Seasonal allergy, |
| | Damp mop (use a damp mop | | | | | Eczema |
| | or wet Swiffer) | | | | | |
| OH.2 | Vacuum, Sweep with a broom, | Weekly | No | No | | Seasonal allergy |
| | Damp mop (use a damp mop | | | | | |
| | or wet Swiffer) | | | | | |
| OH.3 | Vacuum, Sweep with a broom | Weekly | No | No | | Seasonal allergy |
| WA | Vacuum, Damp mop (use a | Weekly | No | No | | Eczema |
| | damp mop or wet Swiffer) | | | | | |
| PA | N/A | N/A | No | No | | N/A |
| KS | Vacuum, Damp mop (use a | Weekly | No | Temporary leak in | Potentially water | Seasonal allergy |
| | damp mop or wet Swiffer) | | | basement that | damaged. However, | |
| | | | | occurs after rains | participant did not | |
| | | | | and dries up within | send us dust from | |
| | | | | 24 hours. | basement, where the | |
| | | | | | temporary leak | |
| | | | | | occurred. | |
| MI | Vacuum, Sweep with a broom | Weekly | No | No | | Seasonal allergy, |
| | | | | | | Eczema |
| CO | Vacuum, Damp mop (use a | Weekly | No | No | | Seasonal allergy |
| | damp mop or wet Swiffer), | | | | | |
| | Electrostatic cloth | | | | | |
| | (e.g., dry Swiffer) | | | | | |
| CA | Vacuum | Twice a | No | No | | Asthma, Seasonal |
| | | month | | | | allergy, Food |
| | | | | | | allergy |

TABLE 9
R² and p-values for statistical tests for gene expression and species abundances. PERMANOVAs (adonis2) were performed
for determining significant differences in gene expression and species composition with ERH condition based on
distance measures. Kruskal-Wallis tests were used for determining significant differences in the number of fungal
annotated genes by ERH and fungal concentration based on ERH. Significant p-values (p < 0.05) are bolded.

| | | | Distance | |
|---|---|---|---|---|
| Data type | Data | Statistical test | type | ERH |
| Microbial function | Gene expression | adonis2 (PERMANOVA) | Euclidean | R² = 0.28 (p ≤ 0.0001) **** |
| Microbial function | Gene expression of fungal annotated genes | adonis2 (PERMANOVA) | Euclidean | R² = 0.34 (p ≤ 0.0001) **** |
| Microbial species | Fungal abundance | adonis2 (PERMANOVA) | Bray-Curtis | R² = 0.21 (p ≤ 0.0001) **** |
| Microbial function | Number of fungal annotated genes present | Kruskal-Wallis | N/A | p = 0.003 ** |
| Microbial species | Fungal concentration | Kruskal-Wallis | N/A | p = 0.0007 *** |

TABLE 10
Differentially abundant species in each ERH condition. Only species that were
significantly abundant were included (FDR-adjusted p < 0.05). The number
of sites that a species was present at each ERH condition are also reported.

| | | | | |
|---|---|---|---|---|
| | | Number | Number | Number |
| | | of sites | of sites | of sites |
| | FDR-adjusted | the species is | the species is | the species is |
| Species | p value | present at 50% | present at 85% | present at 100% |

More abundant at 100% compared to 50%

| | | | | |
|---|---|---|---|---|
| Unknown species | 0.008170816 | 9 | 8 | 9 |
| Chaetomium angustispirale | 0.010680381 | 0 | 0 | 8 |
| Penicillium chrysogenum | 0.030814985 | 2 | 3 | 7 |

More abundant at 100% compared to 85%

| | | | | |
|---|---|---|---|---|
| Unknown species | 0.013221582 | 9 | 8 | 9 |
| Chaetomium angustispirale | 0.010680381 | 0 | 0 | 8 |
| Penicillium chrysogenum | 0.043189707 | 2 | 3 | 7 |
| Naganishia diffluens | 0.043189707 | 4 | 3 | 7 |

More abundant at 50% compared to 100%

| | | | | |
|---|---|---|---|---|
| Unknown species | 0.008170816 | 9 | 8 | 9 |
| Vishniacozyma tephrensis | 0.008170816 | 9 | 7 | 0 |
| Vishniacozyma carnescens | 0.008170816 | 9 | 7 | 0 |
| Vishniacozyma victoriae | 0.008170816 | 9 | 7 | 0 |
| Chaetomium angustispirale | 0.010680381 | 0 | 0 | 8 |
| Neoascochyta desmazieri | 0.010680381 | 8 | 7 | 0 |
| Candida sake | 0.010680381 | 8 | 5 | 0 |
| Vishniacozyma dimennae | 0.010680381 | 8 | 4 | 0 |
| Cryptococcus festucosus | 0.010680381 | 8 | 4 | 0 |
| Cladosporium herbarum | 0.013741654 | 9 | 6 | 1 |
| Tausonia pullulans | 0.013898698 | 7 | 5 | 0 |
| Cyberlindnera jadinii | 0.013898698 | 7 | 5 | 0 |
| Rhodotorula graminis | 0.013898698 | 7 | 5 | 0 |
| Filobasidium wieringae | 0.013898698 | 7 | 5 | 0 |
| Candida tropicalis | 0.013898698 | 7 | 3 | 0 |
| Pyrenochaetopsis leptospora | 0.013898698 | 7 | 3 | 0 |
| Papulaspora byssina | 0.013898698 | 7 | 3 | 0 |
| Lacrymaria glareosa | 0.013898698 | 7 | 2 | 0 |
| Bullera alba | 0.013898698 | 7 | 2 | 0 |
| Buckleyzyma aurantiaca | 0.026981255 | 6 | 5 | 0 |
| Filobasidium magnum | 0.026981255 | 9 | 7 | 1 |
| Papiliotrema flavescens | 0.026981255 | 6 | 4 | 0 |
| Hannaella sinensis | 0.026981255 | 6 | 3 | 0 |
| Pseudorobillarda phragmitis | 0.026981255 | 6 | 3 | 0 |
| Plectosphaerella slobbergiarum | 0.026981255 | 6 | 3 | 0 |
| Kondoa aeria | 0.026981255 | 6 | 3 | 0 |
| Blumeria graminis | 0.026981255 | 6 | 2 | 0 |
| Mrakia frigida | 0.026981255 | 6 | 2 | 0 |
| Periconia celtidis | 0.026981255 | 6 | 2 | 0 |
| Curvibasidium nothofagi | 0.026981255 | 6 | 1 | 0 |
| Geotrichum silvicola | 0.026981255 | 6 | 1 | 0 |
| Penicillium chrysogenum | 0.030814985 | 2 | 3 | 7 |
| Aureobasidium pullulans | 0.039903716 | 8 | 4 | 1 |
| Vermiconia antarctica | 0.043189707 | 5 | 5 | 0 |
| Cladosporium basi-inflatum | 0.043189707 | 9 | 8 | 2 |
| Cystofilobasidium infirmominiatum | 0.043189707 | 5 | 4 | 0 |
| Rhodosporidiobolus colostri | 0.043189707 | 5 | 4 | 0 |
| Hannaella zeae | 0.043189707 | 5 | 3 | 0 |
| Neoascochyta tardicrescens | 0.043189707 | 5 | 3 | 0 |
| Candida parapsilosis | 0.043189707 | 5 | 3 | 0 |
| Coniosporium apollinis | 0.043189707 | 5 | 2 | 0 |
| Symmetrospora oryzicola | 0.043189707 | 5 | 2 | 0 |
| Papiliotrema wisconsinensis | 0.043189707 | 5 | 2 | 0 |
| Cutaneotrichosporon cutaneum | 0.043189707 | 5 | 1 | 0 |
| Arxiella terrestris | 0.043189707 | 5 | 1 | 0 |
| Sporobolomyces patagonicus | 0.043189707 | 5 | 0 | 0 |

More abundant at 50% compared to 85%

| | | | | |
|---|---|---|---|---|
| Sporobolomyces patagonicus | 0.043189707 | 5 | 0 | 0 |

More abundant at 85% compared to 50%

| | | | | |
|---|---|---|---|---|
| Aspergillus ruber | 0.013898698 | 7 | 9 | 1 |
| Aspergillus intermedius | 0.026353673 | 1 | 7 | 2 |

More abundant at 85% compared to 100%

| | | | | |
|---|---|---|---|---|
| Vishniacozyma tephrensis | 0.013898698 | 9 | 7 | 0 |
| Vishniacozyma carnescens | 0.013898698 | 9 | 7 | 0 |
| Vishniacozyma victoriae | 0.013898698 | 9 | 7 | 0 |
| Neoascochyta desmazieri | 0.013898698 | 8 | 7 | 0 |
| Candida sake | 0.043189707 | 8 | 5 | 0 |
| Cladosporium herbarum | 0.047560345 | 9 | 6 | 1 |
| Tausonia pullulans | 0.043189707 | 7 | 5 | 0 |
| Cyberlindnera jadinii | 0.043189707 | 7 | 5 | 0 |
| Rhodotorula graminis | 0.043189707 | 7 | 5 | 0 |
| Filobasidium wieringae | 0.043189707 | 7 | 5 | 0 |
| Buckleyzyma aurantiaca | 0.043189707 | 6 | 5 | 0 |
| Filobasidium magnum | 0.046572571 | 9 | 7 | 1 |
| Vermiconia antarctica | 0.043189707 | 5 | 5 | 0 |
| Aspergillus ruber | 0.010680381 | 7 | 9 | 1 |
| Cystofilobasidium macerans | 0.043189707 | 4 | 5 | 0 |

TABLE 11
Number of fungal annotated genes (BLASTX) present
in each sample based on ERH and Site.

| | Number of fungal | Number of fungal | Number of fungal |
|---|---|---|---|
| | annotated | annotated | annotated |
| | genes present | genes present | genes present |
| Site | at 50% ERH | at 85% ERH | at 100% ERH |
| CA | 25468 | 22260 | 38718 |
| WA | 31127 | 25873 | 39011 |
| CO | 10959 | 24622 | 49614 |
| KS | 36629 | 39676 | 58951 |
| MI | 22798 | 33386 | 49271 |
| OH.1 | 30391 | 34630 | 41166 |
| OH.2 | 34853 | 39845 | 45332 |
| OH.3 | 24784 | 17195 | 26374 |
| PA | 25559 | 43703 | 152419 |

TABLE 12
Number of overall and fungal annotated (BLASTX) upregulated genes
in each ERH comparison. Upregulated genes has a log2FC ≥
2 and were statisticlly significant (FDR-adjusted p value < 0.05).

| | Upregulated | Number of all | Number of fungal |
|---|---|---|---|
| ERH | ERH | upregulated | annotated |
| comparison | condition | genes | upregulated genes |
| 100% vs 85% | 100% | 18266 | 10442 |
| 100% vs 85% | 85% | 5733 | 3285 |
| 100% vs 50% | 100% | 6433 | 4141 |
| 100% vs 50% | 50% | 12979 | 2330 |
| 85% vs 50% | 85% | 21103 | 12845 |
| 85% vs 50% | 50% | 3211 | 710 |

TABLE 13
GO terms associated with fungal growth enriched at each pairwise ERH comparisons. Ontology
categories are BP: Biological Process, CC: Cellular Component, MF: Molecular Function. GO
terms fell into four broad functional categories: Morphological, Stress response, Mitochondria
and Secondary metabolism. All FDR adjusted p-values are significant (padj ≤ 0.05)

| | Ontology | | Broad functional |
|---|---|---|---|
| GO ID | (BP, CC, MF) | GO term | category |
| GO: 0048646 | BP | sporulation resulting in formation of a cellular spore | Morphological |
| GO: 0030435 | BP | conidium formation | Morphological |
| GO: 0043934 | BP | asexual sporulation | Morphological |
| GO: 0048315 | BP | anatomical structure formation involved in morphogenesis | Morphological |
| GO: 0016491 | CC | fungal-type cell wall | Morphological |
| GO: 0098798 | BP | cellular response to farnesol | Stress response |
| GO: 0042438 | BP | mitochondrial electron transport, cytochrome c to oxygen | Mitochondrial |
| GO: 0019594 | CC | mitochondrial protein-containing complex | Mitochondrial |
| GO: 0009277 | MF | oxidoreductase activity | Mitochondrial |
| GO: 0006123 | BP | mannitol metabolic process | Secondary metabolism |
| GO: 0097308 | BP | melanin biosynthetic process | Secondary metabolism |
| GO: 0000909 | BP | sporocarp development involved in sexual reproduction | Morphological |
| GO: 0019954 | BP | asexual reproduction | Morphological |
| GO: 0030436 | BP | asexual sporulation | Morphological |
| GO: 0043934 | BP | sporulation | Morphological |
| GO: 0030435 | BP | sporulation resulting in formation of a cellular spore | Morphological |
| GO: 0042243 | BP | asexual spore wall assembly | Morphological |
| GO: 0030154 | BP | cell differentiation | Morphological |
| GO: 0048646 | BP | anatomical structure formation involved in morphogenesis | Morphological |
| GO: 0009277 | CC | fungal-type cell wall | Morphological |
| GO: 0005992 | BP | trehalose biosynthetic process | Morphological |
| GO: 0070413 | BP | trehalose metabolism in response to stress | Morphological |
| GO: 0097308 | BP | cellular response to farnesol | Stress response |
| GO: 0033554 | BP | cellular response to stress | Stress response |
| GO: 0006123 | BP | mitochondrial electron transport, cytochrome c to oxygen | Mitochondrial |
| GO: 0098798 | CC | mitochondrial protein-containing complex | Mitochondrial |
| GO: 0005751 | CC | mitochondrial respiratory chain complex IV | Mitochondrial |
| GO: 0006839 | BP | mitochondrial transport | Mitochondrial |
| GO: 0016491 | MF | oxidoreductase activity | Mitochondrial |
| GO: 0042438 | BP | melanin biosynthetic process | Secondary metabolism |
| GO: 1900179 | BP | positive regulation of aflatoxin biosynthetic process | Secondary metabolism |
| GO: 0009847 | BP | spore germination | Morphological |
| GO: 0043936 | BP | asexual sporulation resulting in formation of a cellular spore | Morphological |
| GO: 0043937 | BP | regulation of sporulation | Morphological |
| GO: 0048315 | BP | conidium formation | Morphological |
| GO: 0000909 | BP | sporocarp development involved in sexual reproduction | Morphological |
| GO: 0075306 | BP | regulation of conidium formation | Morphological |
| GO: 0000003 | BP | reproduction | Morphological |
| GO: 0030435 | BP | sporulation resulting in formation of a cellular spore | Morphological |
| GO: 0034305 | BP | regulation of asexual sporulation | Morphological |
| GO: 0019954 | BP | asexual reproduction | Morphological |
| GO: 0030582 | BP | reproductive fruiting body development | Morphological |
| GO: 0030436 | BP | asexual sporulation | Morphological |
| GO: 0075259 | BP | spore-bearing structure development | Morphological |
| GO: 0043934 | BP | sporulation | Morphological |
| GO: 0030154 | BP | cell differentiation | Morphological |
| GO: 0030447 | BP | filamentous growth | Morphological |
| GO: 0030448 | BP | hyphal growth | Morphological |
| GO: 0001411 | CC | hyphal tip | Morphological |
| GO: 0030428 | CC | cell septum | Morphological |
| GO: 0140266 | CC | Woronin body | Morphological |
| GO: 0097308 | BP | cellular response to farnesol | Stress response |
| GO: 0033554 | BP | cellular response to stress | Stress response |
| GO: 0098798 | CC | mitochondrial protein-containing complex | Mitochondrial |
| GO: 0033108 | BP | mitochondrial respiratory chain complex assembly | Mitochondrial |
| GO: 0006839 | BP | mitochondrial transport | Mitochondrial |
| GO: 0016491 | MF | oxidoreductase activity | Mitochondrial |
| GO: 0009237 | BP | siderophore metabolic process | Secondary metabolism |
| GO: 0045461 | BP | sterigmatocystin biosynthetic process | Secondary metabolism |
| GO: 0098798 | CC | mitochondrial protein-containing complex | Mitochondrial |
| GO: 0006839 | BP | mitochondrial transport | Mitochondrial |
| GO: 0016491 | MF | oxidoreductase activity | Mitochondrial |

| | |
|---|---|
| None | None |
| None | None |

| | | | False Discovery | Number of |
|---|---|---|---|---|
| GO ID | Enriched at | RH comparison | Rate (FDR) | upregulated genes |
| GO: 0048646 | 100% | 100% vs 85% | 2.93E−02 | 230 |
| GO: 0030435 | 100% | 100% vs 85% | 1.83E−02 | 104 |
| GO: 0043934 | 100% | 100% vs 85% | 1.47E−02 | 122 |
| GO: 0048315 | 100% | 100% vs 85% | 3.70E−03 | 323 |
| GO: 0016491 | 100% | 100% vs 85% | 7.73E−03 | 149 |
| GO: 0098798 | 100% | 100% vs 85% | 1.55E−02 | 37 |
| GO: 0042438 | 100% | 100% vs 85% | 7.80E−03 | 52 |
| GO: 0019594 | 100% | 100% vs 85% | 1.95E−05 | 385 |
| GO: 0009277 | 100% | 100% vs 85% | 1.57E−06 | 2056 |
| GO: 0006123 | 100% | 100% vs 85% | 2.39E−03 | 26 |
| GO: 0097308 | 100% | 100% vs 85% | 1.04E−03 | 34 |
| GO: 0000909 | 100% | 100% vs 50% | 4.41E−02 | 15 |
| GO: 0019954 | 100% | 100% vs 50% | 3.67E−02 | 54 |
| GO: 0030436 | 100% | 100% vs 50% | 2.62E−02 | 47 |
| GO: 0043934 | 100% | 100% vs 50% | 5.09E−04 | 114 |
| GO: 0030435 | 100% | 100% vs 50% | 2.98E−05 | 103 |
| GO: 0042243 | 100% | 100% vs 50% | 2.57E−05 | 11 |
| GO: 0030154 | 100% | 100% vs 50% | 2.12E−03 | 142 |
| GO: 0048646 | 100% | 100% vs 50% | 4.94E−04 | 137 |
| GO: 0009277 | 100% | 100% vs 50% | 1.40E−02 | 62 |
| GO: 0005992 | 100% | 100% vs 50% | 1.42E−02 | 20 |
| GO: 0070413 | 100% | 100% vs 50% | 4.60E−02 | 14 |
| GO: 0097308 | 100% | 100% vs 50% | 1.74E−04 | 22 |
| GO: 0033554 | 100% | 100% vs 50% | 3.48E−02 | 404 |
| GO: 0006123 | 100% | 100% vs 50% | 3.76E−04 | 31 |
| GO: 0098798 | 100% | 100% vs 50% | 9.61E−17 | 214 |
| GO: 0005751 | 100% | 100% vs 50% | 3.90E−04 | 28 |
| GO: 0006839 | 100% | 100% vs 50% | 3.59E−02 | 94 |
| GO: 0016491 | 100% | 100% vs 50% | 2.23E−10 | 814 |
| GO: 0042438 | 100% | 100% vs 50% | 4.78E−04 | 19 |
| GO: 1900179 | 100% | 100% vs 50% | 1.32E−02 | 7 |
| GO: 0009847 | 85% | 85% vs 50% | 3.08E−03 | 32 |
| GO: 0043936 | 85% | 85% vs 50% | 5.45E−04 | 27 |
| GO: 0043937 | 85% | 85% vs 50% | 1.63E−05 | 47 |
| GO: 0048315 | 85% | 85% vs 50% | 6.05E−08 | 102 |
| GO: 0000909 | 85% | 85% vs 50% | 5.91E−08 | 48 |
| GO: 0075306 | 85% | 85% vs 50% | 4.00E−08 | 29 |
| GO: 0000003 | 85% | 85% vs 50% | 3.43E−08 | 160 |
| GO: 0030435 | 85% | 85% vs 50% | 2.89E−08 | 201 |
| GO: 0034305 | 85% | 85% vs 50% | 4.91E−09 | 42 |
| GO: 0019954 | 85% | 85% vs 50% | 5.07E−10 | 146 |
| GO: 0030582 | 85% | 85% vs 50% | 2.18E−10 | 57 |
| GO: 0030436 | 85% | 85% vs 50% | 1.69E−10 | 126 |
| GO: 0075259 | 85% | 85% vs 50% | 7.70E−12 | 63 |
| GO: 0043934 | 85% | 85% vs 50% | 3.47E−13 | 264 |
| GO: 0030154 | 85% | 85% vs 50% | 1.26E−02 | 302 |
| GO: 0030447 | 85% | 85% vs 50% | 1.40E−06 | 153 |
| GO: 0030448 | 85% | 85% vs 50% | 1.89E−08 | 62 |
| GO: 0001411 | 85% | 85% vs 50% | 7.33E−12 | 33 |
| GO: 0030428 | 85% | 85% vs 50% | 1.58E−15 | 152 |
| GO: 0140266 | 85% | 85% vs 50% | 1.07E−06 | 26 |
| GO: 0097308 | 85% | 85% vs 50% | 2.13E−04 | 36 |
| GO: 0033554 | 85% | 85% vs 50% | 2.54E−21 | 1364 |
| GO: 0098798 | 85% | 85% vs 50% | 8.34E−07 | 494 |
| GO: 0033108 | 85% | 85% vs 50% | 8.49E−03 | 109 |
| GO: 0006839 | 85% | 85% vs 50% | 9.99E−04 | 283 |
| GO: 0016491 | 85% | 85% vs 50% | 3.77E−02 | 2069 |
| GO: 0009237 | 85% | 85% vs 50% | 8.62E−03 | 14 |
| GO: 0045461 | 85% | 85% vs 50% | 4.87E−04 | 24 |
| | 85% | 85% vs 100% | 2.27E−06 | 179 |
| | 85% | 85% vs 100% | 4.22E−04 | 105 |
| | 85% | 85% vs 100% | 3.79E−02 | 627 |
| | 50% | 50% vs 100% | N/A | N/A |
| | 50% | 50% vs 85% | N/A | N/A |

TABLE 14
Expression and functions of target genes identified in the study. Targets fell into 3 groups: 1. Upregulated at 100% ERH,
2. Upregulated at 85% and 100% 3. 85% ERH. Target genes are significantly upregulated (log2FC ≥ 5 FDR-adjusted p <
0.001) at 100% ERH or 85% ERH or both. Broad functional categories and GO terms associated with fungal growth are also reported.

| | | | | Number of sites |
|---|---|---|---|---|
| | | | | the gene was |
| | | | | expressed at |
| Trinity gene | Swissprot annotation | Gene | Target gene group | 50% |
| TRINITY_DN51057_c0_g1 | ABR1_ASPFU | abr1 | Upregulated at 100% ERH | 0 |
| TRINITY_DN35090_c0_g1 | ORYZ_EMENI | alp1 | Upregulated at 100% ERH | 0 |
| TRINITY_DN3379_c2_g1 | ALP2_ASPFU | alp2 | Upregulated at 100% ERH | 0 |
| TRINITY_DN8671_c0_g1 | ALTA7_ALTAL | ALTA7 | Upregulated at 100% ERH | 0 |
| TRINITY_DN13900_c0_g2 | ARP1_ASPFU | arp1 | Upregulated at 100% ERH | 0 |
| TRINITY_DN13993_c0_g1 | ARP2_ASPFU | arp2 | Upregulated at 100% ERH | 0 |
| TRINITY_DN60428_c0_g1 | ATFB_ASPPU | atfB | Upregulated at 100% ERH | 0 |
| TRINITY_DN6303_c0_g1 | AYG1_ASPFU | ayg1 | Upregulated at 100% ERH | 0 |
| TRINITY_DN81095_c0_g1 | BRLA_PENCA | brlA | Upregulated at 100% ERH | 0 |
| TRINITY_DN22030_c0_g1 | CATA_ASPFU | catA | Upregulated at 100% ERH | 0 |
| TRINITY_DN19513_c0_g2 | CCG6_NEUCR | ccg-6 | Upregulated at 100% ERH | 0 |
| TRINITY_DN1945_c0_g1 | GEL1_ASPFU | gel1 | Upregulated at 100% ERH | 0 |
| TRINITY_DN8726_c1_g1 | GEL2_ASPFU | gel2 | Upregulated at 100% ERH | 0 |
| TRINITY_DN34293_c0_g1 | GPA3_NEUCR | gna-3 | Upregulated at 100% ERH | 0 |
| TRINITY_DN8361_c0_g1 | HSP30_EMENI | hsp30 | Upregulated at 100% ERH | 0 |
| TRINITY_DN166_c0_g4 | KATG_EMENI | katG | Upregulated at 100% ERH | 0 |
| TRINITY_DN46210_c0_g1 | MPG1_EMENI | mpg1 | Upregulated at 100% ERH | 0 |
| TRINITY_DN18611_c0_g1 | MTLD_PENRW | mtlD | Upregulated at 100% ERH | 0 |
| TRINITY_DN13539_c0_g1 | AP1_EMENI | napA | Upregulated at 100% ERH | 0 |
| TRINITY_DN5789_c0_g1 | NRC2_NEUCR | nrc-2 | Upregulated at 100% ERH | 0 |
| TRINITY_DN5894_c0_g2 | NDUS2_NEUCR | nuo-49 | Upregulated at 100% ERH | 0 |
| TRINITY_DN8019_c0_g1 | KAPR_ASPFU | pkaR | Upregulated at 100% ERH | 0 |
| TRINITY_DN1656_c0_g1 | RODL_EMENI | rodA | Upregulated at 100% ERH | 0 |
| TRINITY_DN17345_c0_g2 | SUN1_ASPFU | sun1 | Upregulated at 100% ERH | 0 |
| TRINITY_DN2023_c0_g1 | TPS1A_ASPFU | tpsA | Upregulated at 100% ERH | 0 |
| TRINITY_DN1053_c0_g4 | VELB_PENRW | velB | Upregulated at 100% ERH | 0 |
| TRINITY_DN2690_c0_g1 | VOSA_PENRW | vosA | Upregulated at 100% ERH | 0 |
| TRINITY_DN372_c0_g1 | WA_EMENI | wA | Upregulated at 100% ERH | 0 |
| TRINITY_DN285_c0_g2 | WETA_PEND2 | wetA | Upregulated at 100% ERH | 0 |
| TRINITY_DN7974_c0_g2 | PP1_EMENI | bimG | Upregulated at 85% and 100% ERH | 0 |
| TRINITY_DN29306_c0_g1 | BIP_ASPNG | bipA | Upregulated at 85% and 100% ERH | 0 |
| TRINITY_DN12451_c0_g1 | CALM_EMENI | camA | Upregulated at 85% and 100% ERH | 0 |
| TRINITY_DN3575_c0_g1 | CALX_ASPFU | Canx homolog | Upregulated at 85% and 100% ERH | 0 |
| TRINITY_DN10372_c0_g1 | CRZA_ASPFU | crzA | Upregulated at 85% and 100% ERH | 0 |
| TRINITY_DN20577_c0_g2 | ECM33_ASPFU | ecm33 | Upregulated at 85% and 100% ERH | 0 |
| TRINITY_DN14038_c0_g1 | GPA1_EMENI | fadA | Upregulated at 85% and 100% ERH | 0 |
| TRINITY_DN8240_c1_g2 | HEX1_EMENI | hex1 | Upregulated at 85% and 100% ERH | 0 |
| TRINITY_DN5762_c0_g1 | MDM10_EMENI | mdm10 | Upregulated at 85% and 100% ERH | 0 |
| TRINITY_DN58717_c0_g1 | NDUS8_NEUCR | nuo21.3c | Upregulated at 85% and 100% ERH | 0 |
| TRINITY_DN11730_c0_g1 | TPIS_EMENI | tpiA | Upregulated at 85% and 100% ERH | 0 |
| TRINITY_DN290926_c0_g1 | ABAA_ASPFU | abaA | Upregulated at 85% ERH | 0 |
| TRINITY_DN2841_c0_g2 | CANIC_EMENI | candA-C | Upregulated at 85% ERH | 0 |
| TRINITY_DN21893_c0_g2 | CANIN_EMENI | candA-N | Upregulated at 85% ERH | 0 |
| TRINITY_DN9867_c0_g2 | CAPZB_ASPFU | cap2 | Upregulated at 85% ERH | 0 |
| TRINITY_DN19748_c0_g1 | CATB_ASPOR | catB | Upregulated at 85% ERH | 0 |
| TRINITY_DN1725_c0_g1 | CCG8_NEUCR | ccg-8 | Upregulated at 85% ERH | 0 |
| TRINITY_DN2314_c0_g2 | CHSA_EMENI | chsA | Upregulated at 85% ERH | 0 |
| TRINITY_DN48838_c0_g1 | CHSC_EMENI | chsC | Upregulated at 85% ERH | 0 |
| TRINITY_DN6916_c0_g1 | DOP1_EMENI | dop1 | Upregulated at 85% ERH | 0 |
| TRINITY_DN11446_c0_g1 | FLBA_EMENI | flbA | Upregulated at 85% ERH | 0 |
| TRINITY_DN219195_c0_g1 | FLUG_EMENI | fluG | Upregulated at 85% ERH | 0 |
| TRINITY_DN4298_c0_g1 | GPAA_ASPFC | gpaA | Upregulated at 85% ERH | 0 |
| TRINITY_DN35531_c0_g1 | GRRA_EMENI | grrA | Upregulated at 85% ERH | 0 |
| TRINITY_DN10742_c0_g1 | HYMA_EMENI | hymA | Upregulated at 85% ERH | 0 |
| TRINITY_DN16965_c0_g1 | LAEA_COCH5 | laeA | Upregulated at 85% ERH | 0 |
| TRINITY_DN10244_c0_g1 | DYHC_EMENI | nudA | Upregulated at 85% ERH | 0 |
| TRINITY_DN15642_c0_g1 | DYL1_EMENI | nudG | Upregulated at 85% ERH | 0 |
| TRINITY_DN15743_c0_g2 | PPOA_EMENI | ppoA | Upregulated at 85% ERH | 0 |
| TRINITY_DN13764_c0_g1 | RHOC_EMENI | rhoC | Upregulated at 85% ERH | 0 |
| TRINITY_DN22023_c0_g1 | DYNA_NEUCR | ro-3 | Upregulated at 85% ERH | 0 |
| TRINITY_DN30562_c0_g6 | SIDA_ASPFU | sidA | Upregulated at 85% ERH | 0 |
| TRINITY_DN3657_c0_g1 | SIDC_ASPFU | sidC | Upregulated at 85% ERH | 0 |
| TRINITY_DN11172_c0_g2 | SIDH_ASPFU | sidH | Upregulated at 85% ERH | 0 |
| TRINITY_DN22889_c0_g3 | CDC45_SCHPO | sna41 | Upregulated at 85% ERH | 0 |
| TRINITY_DN23143_c0_g1 | STCC_EMENI | stcC | Upregulated at 85% ERH | 0 |
| TRINITY_DN4794_c0_g1 | STE12_EMENI | steA | Upregulated at 85% ERH | 0 |
| TRINITY_DN59728_c0_g3 | TCSA_EMENI | tcsA | Upregulated at 85% ERH | 0 |

| | Number of sites | Number of sites the | | | RH comparison |
|---|---|---|---|---|---|
| | the gene was | gene was expressed | | FDR-adjusted | (used for |
| Trinity gene | expressed at 85% | at 100% | log2FC | p-value | log2FC) |
| TRINITY_DN51057_c0_g1 | 0 | 6 | 9.29 | 2.02E−08 | 100% vs 85% |
| TRINITY_DN35090_c0_g1 | 0 | 4 | 25.69 | 1.28E−27 | 100% vs 85% |
| TRINITY_DN3379_c2_g1 | 2 | 7 | 7.15 | 1.82E−08 | 100% vs 85% |
| TRINITY_DN8671_c0_g1 | 9 | 9 | 7.74 | 3.25E−10 | 100% vs 50% |
| TRINITY_DN13900_c0_g2 | 0 | 8 | 12.34 | 7.15E−13 | 100% vs 85% |
| TRINITY_DN13993_c0_g1 | 0 | 8 | 11.9 | 1.07E−16 | 100% vs 85% |
| TRINITY_DN60428_c0_g1 | 0 | 9 | 11.46 | 2.38E−20 | 100% vs 85% |
| TRINITY_DN6303_c0_g1 | 0 | 7 | 10.42 | 5.16E−11 | 100% vs 85% |
| TRINITY_DN81095_c0_g1 | 0 | 3 | 7.39 | 3.79E−06 | 100% vs 85% |
| TRINITY_DN22030_c0_g1 | 1 | 9 | 9.3 | 3.28E−14 | 100% vs 85% |
| TRINITY_DN19513_c0_g2 | 1 | 7 | 8.82 | 1.22E−04 | 100% vs 85% |
| TRINITY_DN1945_c0_g1 | 1 | 7 | 9.39 | 6.82E−06 | 100% vs 50% |
| TRINITY_DN8726_c1_g1 | 2 | 8 | 7.18 | 1.39E−05 | 100% vs 50% |
| TRINITY_DN34293_c0_g1 | 0 | 8 | 9.04 | 9.15E−14 | 100% vs 85% |
| TRINITY_DN8361_c0_g1 | 0 | 9 | 12.11 | 4.52E−25 | 100% vs 85% |
| TRINITY_DN166_c0_g4 | 0 | 7 | 8.97 | 2.03E−11 | 100% vs 85% |
| TRINITY_DN46210_c0_g1 | 2 | 8 | 5.37 | 1.22E−05 | 100% vs 85% |
| TRINITY_DN18611_c0_g1 | 0 | 8 | 11.91 | 8.06E−19 | 100% vs 85% |
| TRINITY_DN13539_c0_g1 | 1 | 5 | 5.4 | 6.36E−04 | 100% vs 85% |
| TRINITY_DN5789_c0_g1 | 0 | 7 | 8.54 | 1.77E−12 | 100% vs 85% |
| TRINITY_DN5894_c0_g2 | 4 | 9 | 5.12 | 1.84E−07 | 100% vs 85% |
| TRINITY_DN8019_c0_g1 | 1 | 8 | 8.47 | 1.39E−13 | 100% vs 85% |
| TRINITY_DN1656_c0_g1 | 2 | 9 | 8.68 | 7.18E−08 | 100% vs 50% |
| TRINITY_DN17345_c0_g2 | 0 | 5 | 8.97 | 7.45E−05 | 100% vs 85% |
| TRINITY_DN2023_c0_g1 | 0 | 8 | 11 | 2.51E−15 | 100% vs 85% |
| TRINITY_DN1053_c0_g4 | 0 | 8 | 8.33 | 1.14E−10 | 100% vs 85% |
| TRINITY_DN2690_c0_g1 | 0 | 8 | 10.35 | 1.02E−12 | 100% vs 85% |
| TRINITY_DN372_c0_g1 | 0 | 9 | 12 | 3.54E−17 | 100% vs 85% |
| TRINITY_DN285_c0_g2 | 0 | 8 | 8.91 | 8.37E−12 | 100% vs 85% |
| TRINITY_DN7974_c0_g2 | 3 | 7 | 29.96 | 1.46E−05 | 100% vs 50% |
| TRINITY_DN29306_c0_g1 | 8 | 6 | 23.19 | 7.44E−05 | 100% vs 50% |
| TRINITY_DN12451_c0_g1 | 5 | 8 | 53.19 | 4.09E−08 | 100% vs 50% |
| TRINITY_DN3575_c0_g1 | 9 | 9 | 50.91 | 1.72E−05 | 100% vs 50% |
| TRINITY_DN10372_c0_g1 | 8 | 8 | 30.39 | 7.49E−07 | 100% vs 50% |
| TRINITY_DN20577_c0_g2 | 7 | 8 | 53.98 | 1.09E−07 | 100% vs 50% |
| TRINITY_DN14038_c0_g1 | 3 | 6 | 17.42 | 3.31E−05 | 100% vs 50% |
| TRINITY_DN8240_c1_g2 | 8 | 8 | 9.09 | 8.67E−05 | 100% vs 50% |
| TRINITY_DN5762_c0_g1 | 4 | 7 | 71.87 | 8.94E−08 | 100% vs 50% |
| TRINITY_DN58717_c0_g1 | 3 | 5 | 20.43 | 1.49E−05 | 100% vs 50% |
| TRINITY_DN11730_c0_g1 | 5 | 9 | 167.76 | 5.63E−11 | 100% vs 50% |
| TRINITY_DN290926_c0_g1 | 4 | 0 | 6.89 | 1.35E−04 | 85% vs 50% |
| TRINITY_DN2841_c0_g2 | 8 | 1 | 10.13 | 1.09E−12 | 85% vs 50% |
| TRINITY_DN21893_c0_g2 | 8 | 1 | 8.63 | 2.81E−10 | 85% vs 50% |
| TRINITY_DN9867_c0_g2 | 9 | 6 | 8.92 | 8.05E−13 | 85% vs 50% |
| TRINITY_DN19748_c0_g1 | 9 | 0 | 12.48 | 4.38E−22 | 85% vs 50% |
| TRINITY_DN1725_c0_g1 | 8 | 1 | 11.01 | 5.41E−14 | 85% vs 50% |
| TRINITY_DN2314_c0_g2 | 9 | 1 | 9.16 | 9.33E−11 | 85% vs 50% |
| TRINITY_DN48838_c0_g1 | 7 | 1 | 8.75 | 2.99E−09 | 85% vs 50% |
| TRINITY_DN6916_c0_g1 | 8 | 3 | 11.59 | 1.33E−13 | 85% vs 50% |
| TRINITY_DN11446_c0_g1 | 8 | 1 | 8.69 | 1.74E−11 | 85% vs 50% |
| TRINITY_DN219195_c0_g1 | 8 | 1 | 10.76 | 4.71E−12 | 85% vs 50% |
| TRINITY_DN4298_c0_g1 | 9 | 9 | 10.18 | 1.92E−22 | 85% vs 50% |
| TRINITY_DN35531_c0_g1 | 7 | 1 | 9.23 | 7.81E−10 | 85% vs 50% |
| TRINITY_DN10742_c0_g1 | 7 | 1 | 9.83 | 2.18E−09 | 85% vs 50% |
| TRINITY_DN16965_c0_g1 | 8 | 1 | 11.65 | 1.94E−16 | 85% vs 50% |
| TRINITY_DN10244_c0_g1 | 9 | 4 | 11.5 | 1.02E−16 | 85% vs 50% |
| TRINITY_DN15642_c0_g1 | 8 | 3 | 11.04 | 1.56E−12 | 85% vs 50% |
| TRINITY_DN15743_c0_g2 | 3 | 0 | 6.72 | 4.08E−04 | 85% vs 50% |
| TRINITY_DN13764_c0_g1 | 9 | 1 | 9.56 | 8.36E−15 | 85% vs 50% |
| TRINITY_DN22023_c0_g1 | 5 | 0 | 7.96 | 8.91E−06 | 85% vs 50% |
| TRINITY_DN30562_c0_g6 | 4 | 0 | 7.91 | 1.80E−05 | 85% vs 50% |
| TRINITY_DN3657_c0_g1 | 7 | 1 | 8.43 | 2.63E−10 | 85% vs 50% |
| TRINITY_DN11172_c0_g2 | 8 | 1 | 11.03 | 2.38E−15 | 85% vs 50% |
| TRINITY_DN22889_c0_g3 | 5 | 1 | 8.01 | 1.72E−06 | 85% vs 50% |
| TRINITY_DN23143_c0_g1 | 4 | 0 | 8.78 | 4.18E−06 | 85% vs 50% |
| TRINITY_DN4794_c0_g1 | 9 | 0 | 11.33 | 5.63E−18 | 85% vs 50% |
| TRINITY_DN59728_c0_g3 | 4 | 2 | 5.51 | 3.83E−04 | 85% vs 50% |

| Trinity gene | Broad Functional Category | GO ID |
|---|---|---|
| TRINITY_DN51057_c0_g1 | Morphological, Secondary metabolism | GO: 0016491, GO: 0042438 |
| TRINITY_DN35090_c0_g1 | Morphological | GO: 0005576 |
| TRINITY_DN3379_c2_g1 | Morphological | GO: 0048646, GO: 0030436, GO: 0030435 |
| TRINITY_DN8671_c0_g1 | Stress response | GO: 0005737 |
| TRINITY_DN13900_c0_g2 | Morphological, Secondary metabolism | GO: 0042438, GO: 0048646, GO: 0030436, GO: 0048315, |
| | | GO: 0030435 |
| TRINITY_DN13993_c0_g1 | Stress response, Secondary | GO: 0016491, GO: 0042438 |
| | metabolism | |
| TRINITY_DN60428_c0_g1 | Secondary metabolism | GO: 1900179 |
| TRINITY_DN6303_c0_g1 | Secondary metabolism | GO: 0042438 |
| TRINITY_DN81095_c0_g1 | Morphological | GO: 0048646, GO: 0030436, GO: 0048315, GO: 0030435 |
| TRINITY_DN22030_c0_g1 | Morphological, Stress response | GO: 0016491 |
| TRINITY_DN19513_c0_g2 | Morphological | GO: 0009277 |
| TRINITY_DN1945_c0_g1 | Morphological | GO: 0009277 |
| TRINITY_DN8726_c1_g1 | Morphological | GO: 0009277 |
| TRINITY_DN34293_c0_g1 | Morphological | GO: 0048646, GO: 0030436, GO: 0048315, GO: 0030435 |
| TRINITY_DN8361_c0_g1 | Stress response | GO: 0097308 |
| TRINITY_DN166_c0_g4 | Stress response, Secondary | GO: 0016491 |
| | metabolism | |
| TRINITY_DN46210_c0_g1 | Morphological | GO: 0043934 |
| TRINITY_DN18611_c0_g1 | Stress response, Secondary | GO: 0016491, GO: 0019594 |
| | metabolism | |
| TRINITY_DN13539_c0_g1 | Morphological, Stress response | GO: 0048646, GO: 0030436, GO: 0030435 |
| TRINITY_DN5789_c0_g1 | Morphological | GO: 0048646, GO: 0030436, GO: 0048315, GO: 0030435 |
| TRINITY_DN5894_c0_g2 | Mitochondria | GO: 0098798 |
| TRINITY_DN8019_c0_g1 | Morphological | GO: 0048646, GO: 0030436, GO: 0030435 |
| TRINITY_DN1656_c0_g1 | Morphological | GO: 0042243, GO: 0048646, GO: 0009277 |
| TRINITY_DN17345_c0_g2 | Morphological | GO: 0009277 |
| TRINITY_DN2023_c0_g1 | Morphological, Stress response | GO: 0005992, GO: 0070413 |
| TRINITY_DN1053_c0_g4 | Morphological, Secondary metabolism | GO: 0048646, GO: 0030435 |
| TRINITY_DN2690_c0_g1 | Morphological, Secondary metabolism | GO: 0048646, GO: 0030436, GO: 0048315, GO: 0030435 |
| TRINITY_DN372_c0_g1 | Morphological, Secondary metabolism | GO: 0016491, GO: 0048646, GO: 0030436, GO: 0048315, |
| | | GO: 0030435 |
| TRINITY_DN285_c0_g2 | Morphological | GO: 0048646, GO: 0030436, GO: 0048315, GO: 0030435 |
| TRINITY_DN7974_c0_g2 | Morphological, Stress response | GO: 0030435, GO: 0048646, GO: 0043934, GO: 0030154, |
| | | GO: 0033554, GO: 0030428 |
| TRINITY_DN29306_c0_g1 | Stress response | GO: 0033554 |
| TRINITY_DN12451_c0_g1 | Morphological | GO: 0001411, GO: 0009847 |
| TRINITY_DN3575_c0_g1 | Stress response | GO: 0033554 |
| TRINITY_DN10372_c0_g1 | Morphological | GO: 0043934, GO: 0030436, GO: 0019954, GO: 0000003, |
| | | GO: 0048315, GO: 0009847 |
| TRINITY_DN20577_c0_g2 | Morphological | GO: 0009277 |
| TRINITY_DN14038_c0_g1 | Morphological, Secondary metabolism | GO: 0043934, GO: 0030436, GO: 0019954, GO: 0000909, |
| | | GO: 0075259, GO: 0030582, GO: 0034305, GO: 0000003, |
| | | GO: 0075306, GO: 0048315, GO: 0043937, GO: 0045461 |
| TRINITY_DN8240_c1_g2 | Morphological | GO: 0140266 |
| TRINITY_DN5762_c0_g1 | Mitochondria, Stress response | GO: 0098798, GO: 0097308, GO: 0006839 |
| TRINITY_DN58717_c0_g1 | Mitochondria | GO: 0098798, GO: 0016491, GO: 0033108 |
| TRINITY_DN11730_c0_g1 | Stress response | GO: 0097308 |
| TRINITY_DN290926_c0_g1 | Morphological | GO: 0043934, GO: 0075259, GO: 0030436, GO: 0019954, |
| | | GO: 0034305, GO: 0030435, GO: 0000003, GO: 0048315, |
| | | GO: 0043937, GO: 0043936, GO: 0030154 |
| TRINITY_DN2841_c0_g2 | Morphological | GO: 0043934, GO: 0075259, GO: 0030582, GO: 0030435, |
| | | GO: 0000909, GO: 0030154 |
| TRINITY_DN21893_c0_g2 | Morphological | GO: 0043934, GO: 0075259, GO: 0030582, GO: 0030435, |
| | | GO: 0000909, GO: 0030154 |
| TRINITY_DN9867_c0_g2 | Morphological | GO: 0030447 |
| TRINITY_DN19748_c0_g1 | Stress response | GO: 0033554 |
| TRINITY_DN1725_c0_g1 | Stress response | GO: 0033554 |
| TRINITY_DN2314_c0_g2 | Morphological | GO: 0030428, GO: 0043934, GO: 0030436, GO: 0019954, |
| | | GO: 0000003, GO: 0048315 |
| TRINITY_DN48838_c0_g1 | Morphological | GO: 0030428, GO: 0043934, GO: 0001411, GO: 0030436, |
| | | GO: 0019954, GO: 0030448, GO: 0000003, GO: 0048315, |
| | | GO: 0030447 |
| TRINITY_DN6916_c0_g1 | Morphological | GO: 0043934, GO: 0075259, GO: 0030436, GO: 0030582, |
| | | GO: 0019954, GO: 0034305, GO: 0000003, GO: 0075306, |
| | | GO: 0000909, GO: 0048315, GO: 0043937 |
| TRINITY_DN11446_c0_g1 | Morphological, Secondary metabolism | GO: 0043934, GO: 0030436, GO: 0019954, GO: 0034305, |
| | | GO: 0000003, GO: 0075306, GO: 0048315, GO: 0043937, |
| | | GO: 0045461 |
| TRINITY_DN219195_c0_g1 | Morphological, Secondary metabolism | GO: 0043934, GO: 0075259, GO: 0030436, GO: 0030582, |
| | | GO: 0019954, GO: 0034305, GO: 0030435, GO: 0000003, |
| | | GO: 0075306, GO: 0048315, GO: 0043937, GO: 0045461, |
| | | GO: 0030154 |
| TRINITY_DN4298_c0_g1 | Morphological, Secondary metabolism | GO: 0043934, GO: 0075259, GO: 0030436, GO: 0030582, |
| | | GO: 0019954, GO: 0034305, GO: 0000003, GO: 0075306, |
| | | GO: 0000909, GO: 0048315, GO: 0043937, GO: 0045461 |
| TRINITY_DN35531_c0_g1 | Morphological | GO: 0043934, GO: 0030435, GO: 0030154 |
| TRINITY_DN10742_c0_g1 | Morphological | GO: 0043934, GO: 0030436, GO: 0019954, GO: 0030448, |
| | | GO: 0000003, GO: 0048315, GO: 0030447 |
| TRINITY_DN16965_c0_g1 | Morphological, Secondary metabolism | GO: 0043934, GO: 0030435, GO: 0045461, GO: 0030154 |
| TRINITY_DN10244_c0_g1 | Morphological | GO: 0030428, GO: 0001411 |
| TRINITY_DN15642_c0_g1 | Morphological | GO: 0043934, GO: 0030436, GO: 0019954, GO: 0030435, |
| | | GO: 0000003, GO: 0048315, GO: 0030154 |
| TRINITY_DN15743_c0_g2 | Morphological, Secondary metabolism | GO: 0043934, GO: 0030436, GO: 0019954, GO: 0034305, |
| | | GO: 0030435, GO: 0000003, GO: 0043937, GO: 0045461, |
| | | GO: 0043936, GO: 0030154 |
| TRINITY_DN13764_c0_g1 | Morphological | GO: 0030428 |
| TRINITY_DN22023_c0_g1 | Morphological | GO: 0030428 |
| TRINITY_DN30562_c0_g6 | Stress response, Secondary | GO: 0033554, GO: 0009237 |
| | metabolism | |
| TRINITY_DN3657_c0_g1 | Stress response, Secondary | GO: 0033554, GO: 0009237 |
| | metabolism | |
| TRINITY_DN11172_c0_g2 | Stress response | GO: 0033554 |
| TRINITY_DN22889_c0_g3 | Stress response | GO: 0033554 |
| TRINITY_DN23143_c0_g1 | Secondary metabolism | GO: 0045461 |
| TRINITY_DN4794_c0_g1 | Morphological | GO: 0075259, GO: 0030582, GO: 0000003, GO: 0000909 |
| TRINITY_DN59728_c0_g3 | Morphological | GO: 0043934, GO: 0030436, GO: 0019954, GO: 0034305, |
| | | GO: 0030435, GO: 0000003, GO: 0075306, GO: 0048315, |
| | | GO: 0043937, GO: 0030154 |

### REFERENCES

1. Abbas H K, Egley G H, Paul R N. Effect of conidia production temperature on germination and infectivity of Alternaria helianthi. Phytopathology. 1995; 85:667-82.
2. Adams R I, Sylvain I, Spilak M P, Taylor J W, Waring M S, Mendell M J. Fungal signature of moisture damage in buildings: Identification by targeted and untargeted approaches with mycobiome data. Appl Environ Microbiol [Internet]. 2020; 86. Available from: http://dx.doi.org/10.1128/AEM.01047-20
3. Adan, O. C. G., On the fungal defacement of interior finishes. 1994, Eindhoven: Eindhoven University of Technology.
4. Adan, O. G. C. and R. A. Samson, Fundamentals of mold growth in indoor environments and strategies for healthy living. 2011, The Netherlands: Wageningen Academic Publishers.
5. Agarwal R. Severe asthma with fungal sensitization. Curr Allergy Asthma Rep. 2011; 11:403-13.
6. Aimanianda V, Bayry J, Bozza S, Kniemeyer O, Perruccio K, Elluru S R, et al. Surface hydrophobin prevents immune recognition of airborne fungal spores. Nature. 2009; 460:1117-21.
7. Al horr, Y., M. Arif, M. Katafygiotou, A. Mazroei, A. Kaushik, and E. Elsarrag, Impact of indoor environmental quality on occupant well-being and comfort: A review of the literature. International Journal of Sustainable Built Environment, 2016. 5(1): p. 1-11.
8. Amend A S, Seifert K A, Samson R, Bruns T D. Indoor fungal composition is geographically patterned and more diverse in temperate zones than in the tropics. Proc Natl Acad Sci U S A. 2010; 107:13748-53.
9. Andersen, M. R., J. B. Nielsen, A. Klitgaard, L. M. Petersen, M. Zachariasen, T. J. Hansen, L. H. Blicher, C. H. Gotfredsen, T. O. Larsen, K. F. Nielsen, and U. H. Mortensen, Accurate prediction of secondary metabolite gene clusters in filamentous fungi. Proceedings of the National Academy of Sciences of the United States of America, 2013. 110(1): p. E99-E107.
10. Ashburner M, Ball C A, Blake J A, Botstein D, Butler H, Cherry J M, et al. Gene ontology: tool for the unification of biology. The Gene Ontology Consortium. Nat Genet. 2000; 25:25-9.
11. Babraham Bioinformatics-FastQC A quality control tool for high throughput sequence data [Internet]. Accessed v0.11.9 on Aug. 14, 2022. 2010 [cited 2022 Aug. 14]. Available from: http://www.bioinformatics.babraham.ac.uk/projects/fastqc/12.
Barnes C, Portnoy J M, Ciaccio C E, Pacheco F. A comparison of subject room dust with home vacuum dust for evaluation of dust-borne aeroallergens. Ann Allergy Asthma Immunol. 2013; 110:375-9.
13. Basu T, Seyedmousavi S, Sugui J A, Balenga N, Zhao M, Kwon Chung K J, et al. Aspergillus fumigatus alkaline protease 1(Alp1/Asp f13) in the airways correlates with asthma severity. J Allergy Clin Immunol. 2018; 141:423-425.e7.
14. Benjamini Y, Hochberg Y. Controlling the false discovery rate: A practical and powerful approach to multiple testing. J R Stat Soc. 1995; 57:289-300.
15. Bertolini P, Tian S P. Effect of temperature of production of Botrytis allii conidia on their pathogenicity to harvested white onion bulbs. Plant Pathol. 1997; 46:432-8.
16. Bettina N S, Webster D G. Microbial indicators as a diagnostic tool for assessing water quality and climate stress in coral reef ecosystems. Marine Biology. 2017; 164:1-18.
17. Bo L, Dewey C N. RSEM: accurate transcript quantification from RNA-Seq data with or without a reference genome. BMC Bioinformatics. 2011; 12.
18. Bolger A M, Lohse M, Usadel B. Trimmomatic: a flexible trimmer for Illumina sequence data. Bioinformatics. 2014; 30:2114-20.
19. Bope A, Haines S R, Hegarty B, Weschler C J, Peccia J, Dannemiller K C. Degradation of phthalate esters in floor dust at elevated relative humidity. Environ Sci Process Impacts. 2019; 21:1268-79.
20. Bope, A., M. H. Weir, A. Pruden, M. Morowitz, J. Mitchell, and K. C. Dannemiller, Translating research to policy at the NCSE 2017 symposium “Microbiology of the Built Environment: Implications for Health and Design”. Microbiome, 2018. 6: p. 160.
21. Bope, A., S. R. Haines, B. Hegarty, C. J. Weschler, J. Peccia, and K. C. Dannemiller, Degradation of phthalate esters in floor dust at elevated relative humidity. Environ Sci Process Impacts, 2019.
22. Boutet E, Lieberherr D, Tognolli M, Schneider M, Bansal P, Bridge A J, et al. UniProtKB/Swiss-Prot, the manually annotated section of the UniProt KnowledgeBase: How to use the entry view. Methods Mol Biol. 2016; 1374:23-54.
23. Bridge, P. and B. Spooner, Soil fungi: diversity and detection. Plant and Soil, 2001. 232(1): p. 147-154.
24. Bryant D M, Johnson K, DiTommaso T, Tickle T, Couger M B, Payzin-Dogru D, et al. A tissue-mapped axolotl D E Novo transcriptome enables identification of limb regeneration factors. Cell Rep. 2017; 18:762-76.
25. Bryant-Stephens T. Asthma disparities in urban environments. J Allergy Clin Immunol. 2009; 123:1199-206; quiz 1207-8.
26. Burke C, Steinberg P, Rusch D, Kjelleberg S, Thomas T. Bacterial community assembly based on functional genes rather than species. Proc Natl Acad Sci USA. 2011; 108:14288-93.
27. Calderone R, Li D, Traven A. System-level impact of mitochondria on fungal virulence: to metabolism and beyond. FEMS Yeast Res. 2015; 15: fov027.
28. Callahan B J, McMurdie P J, Rosen M J, Han A W, Johnson A J A, Holmes S P. DADA2: High-resolution sample inference from Illumina amplicon data. Nat Methods. 2016; 13:581-3.
29. Camacho C, Coulouris G, Avagyan V, Ma N, Papadopoulos J, Bealer K, et al. BLAST+: architecture and applications. BMC Bioinformatics. 2009; 10:421.
30. Carbon S, Ireland A, Mungall C J, Shu S, Marshall B, Lewis S, et al. AmiGO: online access to ontology and annotation data. Bioinformatics. 2009; 25:288-9.
31. Castner, J., G. E. Gehrke, N. Shapiro, and K. C. Dannemiller, Community interest and feasibility of using a novel smartphone-based formaldehyde exposure detection technology. Public Health Nurs, 2018. 35(4): p. 261-272.
32. Choi H, Byrne S, Larsen L S, Sigsgaard T, Thorne P S, Larsson L, et al. Residential culturable fungi, (1-3, 1-6)-β-d-glucan, and ergosterol concentrations in dust are not associated with asthma, rhinitis, or eczema diagnoses in children. Indoor Air. 2014; 24:158-70.
33. Cramer R A. Secretion stress and fungal pathogenesis: A new, exploitable chink in fungal armor? Virulence. 2011. p. 1-3.
34. Crocker, D. D., S. Kinyota, G. G. Dumitru, C. B. Ligon, E. J. Herman, J. M. Ferdinands, D. P. Hopkins, B. M. Lawrence, and T. A. Sipe, Effectiveness of Home-Based, Multi-Trigger, Multicomponent Interventions with an Environmental Focus for Reducing Asthma Morbidity: A Community Guide Systematic Review. American Journal of Preventive Medicine, 2011. 41(2, Supplement 1): p. S5-S32.
35. Croft C A, Culibrk L, Moore M M, Tebbutt S J. Interactions of Aspergillus fumigatus conidia with airway epithelial cells: A critical review. Front Microbiol. 2016; 7:472.
36. Dannemiller K C, Gent J F, Leaderer B P, Peccia J. Influence of housing characteristics on bacterial and fungal communities in homes of asthmatic children. Indoor Air. 2016; 26:179-92.
37. Dannemiller K C, Weschler C J, Peccia J. Fungal and bacterial growth in floor dust at elevated relative humidity levels. Indoor Air. 2017; 27:354-63.
38. Dannemiller, K., M. J. Mendell, J. M. Macher, K. Kumagai, A. Bradman, N. Holland, K. Harley, B. Eskenazi, and J. Peccia, Next-generation DNA sequencing reveals that low fungal diversity in house dust is associated with childhood asthma development. Indoor Air, 2014. 24(3): p. 236-247.
39. Dannemiller, K. C., D. Reeves, K. Bibby, N. Yamamoto, and J. Peccia, Fungal High-throughput Taxonomic Identification tool for use with Next-Generation Sequencing (FHiTINGS). Journal of Basic Microbiology, 2013. 54(4): p. 315-321.
40. Dannemiller, K. C., Engineering Design for Environmental Health: A New Course Preparing Students to Address Interdisciplinary Challenges. Environmental Engineering Science, 2019. 36(2): p. 257-261.
41. Dannemiller, K. C., J. F. Gent, B. P. Leaderer, and J. Peccia, Indoor microbial communities: influence on asthma severity in atopic and non-atopic children. Journal of Allergy and Clinical Immunology, 2016: p. doi: 10.1016/j.jaci.2015.11.027.
42. Dannemiller, K. C., J. S. Murphy, S. L. Dixon, K. G. Pennell, E. M. Suuberg, D. E. Jacobs, and M. Sandel, Formaldehyde concentrations in household air of asthma patients determined using colorimetric detector tubes. Indoor Air, 2013. 23(4): p. 285-294.
43. Dannemiller, K. C., Moving towards a Robust Definition for a “Healthy” Indoor Microbiome. mSystems, 2019. 4(3): p. e00074-19.
44. Dannemiller, K. C., N. Lang-Yona, N. Yamamoto, Y. Rudich, and J. Peccia, Combining real-time PCR and next-generation DNA sequencing to provide quantitative comparisons of fungal aerosol populations. Atmospheric Environment, 2014. 84: p. 113-121.
45. Darzi Y, Letunic I, Bork P, Yamada T. iPath3.0: interactive pathways explorer v3. Nucleic Acids Res. 2018; 46: W510-3.
46. Davidson N M, Oshlack A. Corset: enabling differential gene expression analysis for de novo assembled transcriptomes. Genome Biol. 2014; 15:410.
47. De Lucca, S. D., D. J. M. Taylor, T. J. O'Meara, A. S. Jones, and E. R. Tovey, Measurement and characterization of cockroach allergens detected during normal domestic activity. Journal of Allergy and Clinical Immunology, 1999. 104(3): p. 672-680.
48. Dixon P. VEGAN, a package of R functions for community ecology. J Veg Sci. 2003; 14:927-30.
49. Dunn, R. R., N. Fierer, J. B. Henley, J. W. Leff, and H. L. Menninger, Home Life: Factors Structuring the Bacterial Diversity Found within and between Homes. PLOS ONE, 2013. 8(5): p. e64133.
50. EPA, U., Mold Course Chapter 2: Why and Where Mold Grows.
51. Escalas A, Hale L, Voordeckers J W, Yang Y, Firestone M K, Alvarez-Cohen L, et al. Microbial functional diversity: From concepts to applications. Ecol Evol. 2019; 9:12000-16.
52. Estokova, A. and M. Balintova, Advances in Environmental Engineering. 2018, Multidisciplinary Digital Publishing Institute.
53. Fisk W J, Eliseeva E A, Mendell M J. Association of residential dampness and mold with respiratory tract infections and bronchitis: a meta-analysis. Environ Health. 2010; 9:72.
54. Fisk W J, Lei-Gomez Q, Mendell M J. Meta-analyses of the associations of respiratory health effects with dampness and mold in homes. Indoor Air. 2007; 17:284-96.
55. Frankel M, Bekö G, Timm M, Gustavsen S, Hansen E W, Madsen A M. Seasonal variations of indoor microbial exposures and their relation to temperature, relative humidity, and air exchange rate. Appl Environ Microbiol. 2012; 78:8289-97.
56. Fu L, Niu B, Zhu Z, Wu S, Li W. C D-HIT: accelerated for clustering the next-generation sequencing data. Bioinformatics. 2012; 28:3150-2.
57. Gemene, K. L. and M. E. Meyerhoff, Detection of protease activities by flash chronopotentiometry using a reversible polycation-sensitive polymeric membrane electrode. Analytical biochemistry, 2011. 416(1): p. 67-73.
58. Grabherr M G, Haas B J, Yassour M, Levin J Z, Thompson D A, Amit I, et al. Full-length transcriptome assembly from RNA-Seq data without a reference genome. Nat Biotechnol. 2011; 29:644-52.
59. Green, B. J., E. R. Tovey, J. K. Sercombe, F. M. Blachere, D. H. Beezhold, and D. Schmechel, Airborne fungal fragments and allergenicity. Medical Mycology, 2006. 44(Supplement_1): p. S245-S255.
60. Gu Z, Eils R, Schlesner M. Complex heatmaps reveal patterns and correlations in multidimensional genomic data. Bioinformatics. 2016; 32:2847-9.
61. Haas B J. TransDecoder (Find Coding Regions Within Transcripts). Accessed TransDecoder-v5.5.0 on Oct. 9, 2022 [Internet]. 2017 [cited 2022 Oct. 9]. Available from: https://github.com/TransDecoder/TransDecoder
62. Haines S R, Hall E C, Marciniak K, Misztal P K, Goldstein A H, Adams R I, et al. Microbial growth and volatile organic compound (VOC) emissions from carpet and drywall under elevated relative humidity conditions. Microbiome. 2021; 9:209.
63. Haines S R, Siegel J A, Dannemiller K C. Modeling microbial growth in carpet dust exposed to diurnal variations in relative humidity using the “Time-of-Wetness” framework. Indoor Air. 2020; 30:978-92.
64. Harris, S. D., A. F. Hofmann, H. W. Tedford, and M. P. Lee, Identification and Characterization of Genes Required for Hyphal Morphogenesis in the Filamentous Fungus <em>Aspergillus nidulans</em>. Genetics, 1999. 151(3): p. 1015-1025.
65. He Z, Zhang P, Wu L, Rocha A M, Tu Q, Shi Z, et al. Microbial functional gene diversity predicts groundwater contamination and ecosystem functioning. MBio [Internet]. 2018; 9. Available from: http://dx.doi.org/10.1128/mBio.02435-17
66. Hegarty B, Dannemiller K C, Peccia J. Gene expression of indoor fungal communities under damp building conditions: Implications for human health. Indoor Air. 2018; 28:548-58.
67. Hirabayashi, Y., R. Mahendran, S. Koirala, L. Konoshima, D. Yamazaki, S. Watanabe, H. Kim, and S. Kanae, Global flood risk under climate change. Nature Climate Change, 2013. 3: p. 816.
68. Hospodsky, D., J. Qian, W. W. Nazaroff, N. Yamamoto, K. Bibby, H. Rismani-Yazdi, and J. Peccia, Human occupancy as a source of indoor airborne bacteria. PLOS One, 2012. 7.
69. IOM (Institute of Medicine). Damp indoor spaces and health. Washington D. C: The National Academic Press; 2004.
70. Iossifova Y Y, Reponen T, Ryan P H. Mold exposure during in-fancy as a predictor of potential asthma development. Ann Allergy Asthma Immunol. 2009; 102:131-7.
71. Jaakkola M S, Quansah R, Hugg T T, Heikkinen S A M, Jaakkola J J K. Association of indoor dampness and molds with rhinitis risk: a systematic review and meta-analysis. J Allergy Clin Immunol. 2013; 132:1099-1110.e18.
72. Jiang Y, Xiong X, Danska J, Parkinson J. Metatranscriptomic analysis of diverse microbial communities reveals core metabolic pathways and microbiome-specific functionality. Microbiome. 2016; 4:2.
73. Kanchongkittiphon W, Mendell M J, Gaffin J M, Wang G, Phipatanakul W. Indoor environmental exposures and asthma exac-erbation: an update to the 2000 review by the Institute of. Medicine Environ Health Perspect. 2015; 123:6-20.
74. Kanehisa M, Goto S, Furumichi M, Tanabe M, Hirakawa M. KEGG for representation and analysis of molecular networks involving diseases and drugs. Nucleic Acids Res. 2010; 38: D355-60.
75. Kanehisa M, Sato Y. KEGG Mapper for inferring cellular functions from protein sequences. Protein Sci. 2020; 29:28-35.
76. Karen C. Dannemiller, Luis Acosta, Adnan Divjan, Andrew G. Rundle, Rachel L. Miller, Angela Lemons, Brett J. Green, and M. S. Perzanowski. Domestic fungal diversity in NYC homes, neighborhood asthma prevalence and asthma persistence. in American Academy of Allergy, Asthma, and Immunology. 2018. Orlando, F L.
77. Keller N P, Turner G, Bennett J W. Fungal secondary metabolism—from biochemistry to genomics. Nat Rev Microbiol. 2005; 3:937-47.
78. Kheradmand, F., A. Kiss, J. Xu, S. H. Lee, P. E. Kolattukudy, and D. B. Corry, A protease-activated pathway underlying Th cell type 2 activation and allergic lung disease. J Immunol, 2002. 169(10): p. 5904-11.
79. Klepeis, N. E., W. C. Nelson, W. R. Ott, J. P. Robinson, A. M. Tsang, P. Switzer, J. V. Behar, S. C. Hern, and W. H. Engelmann, The National Human Activity Pattern Survey (NHAPS): a resource for assessing exposure to environmental pollutants. Journal of Exposure Analysis & Environmental Epidemiology, 2001. 11(3): p. 231.
80. Korpi, A., A. L. Pasanen, P. Pasanen, and P. Kalliokoski, Microbial growth and metabolism in house dust. International Biodeterioration & Biodegradation, 1997. 40(1): p. 19-27.
81. Langmead B, Salzberg S L. Fast gapped-read alignment with Bowtie 2. Nat Methods. 2012; 9:357-9.
82. Lang-Yona N, Shuster-Meiseles T, Mazar Y, Yarden O, Rudich Y. Impact of urban air pollution on the allergenicity of Aspergillus fumigatus conidia: Outdoor exposure study supported by laboratory experiments. Sci Total Environ. 2016; 541:365-71.
83. Lanphear, B. P., M. Emond, D. E. Jacobs, M. Weitzman, M. Tanner, N. L. Winter, B. Yakir, and S. Eberly, A Side-by-Side Comparison of Dust Collection Methods for Sampling Lead-Contaminated House Dust. Environmental Research, 1995. 68(2): p. 114-123.
84. Lanphear, B. P., R. S. Kahn, O. Berger, P. Auinger, S. M. Bortnick, and R. W. Nahhas, Contribution of Residential Exposures to Asthma in U S Children and Adolescents. Pediatrics, 2001. 107(6): p. e98-e98.
85. Lanphear, B. P., T. D. Matte, J. Rogers, R. P. Clickner, B. Dietz, R. L. Bornschein, P. Succop, K. R. Mahaffey, S. Dixon, W. Galke, M. Rabinowitz, M. Farfel, C. Rohde, J. Schwartz, P. Ashley, and D. E. Jacobs, The Contribution of Lead-Contaminated House Dust and Residential Soil to Children's Blood Lead Levels. Environmental Research, 1998. 79(1): p. 51-68.
86. Law C W, Alhamdoosh M, Su S, Dong X, Tian L, Smyth G K, et al. RNA-seq analysis is easy as 1-2-3 with limma, Glimma and edgeR. F1000Res. 2016; 5:1408.
87. Lax S, Smith D P, Hampton-Marcell J, Owens S M, Handley K M, Scott N M, et al. Longitudinal analysis of microbial interaction between humans and the indoor environment. Science. 2014; 345:1048-52.
88. Li H Z, Gao X, Li X Y, Chen Q J, Dong J, Zhao W C. Evaluation of assembly strategies using RNA-Seq data associated with grain development of wheat (Triticum aestivum L.). PLoS ONE. 2013; 8.
89. Li W, Godzik A. Cd-hit: a fast program for clustering and comparing large sets of protein or nucleotide sequences. Bioinformatics. 2006; 22:1658-9.
90. Linder M B, Szilvay G R, Nakari-Setälä T, Penttilä M E. Hydrophobins: the protein-amphiphiles of filamentous fungi. FEMS Microbiol Rev. 2005; 29:877-96.
91. Love M I, Huber W, Anders S. Moderated estimation of fold change and dispersion for RNA-seq data with DESeq2. Genome Biol. 2014; 15:550.
92. Low S Y, Dannemiller K, Yao M, Yamamoto N, Peccia J. The allergenicity of Aspergillus fumigatus conidia is influenced by growth temperature. Fungal Biol. 2011; 115:625-32.
93. Ma X, Zhang Q, Zheng M, Gao Y, Yuan T, Hale L, et al. Microbial functional traits are sensitive indicators of mild disturbance by lamb grazing. ISME J. 2019; 13:1370-3.
94. Marcet-Houben M, Gabaldón T. Evolutionary and functional patterns of shared gene neighbourhood in fungi. Nat Microbiol. 2019; 4:2383-92.
95. Martin M. Cutadapt removes adapter sequences from high-throughput sequencing reads. EMBnet J. 2011; 17:10.
96. Meadow J F, Altrichter A E, Kembel S W, Kline J, Mhuireach G, Moriyama M, et al. Indoor airborne bacterial communities are influenced by ventilation, occupancy, and outdoor air source. Indoor Air. 2014; 24:41-8.
97. Means, B., H. Wang, X. Wei, S. Lynch, V. Peters, V. Young, and C. Allen, Expanding STEM opportunities through inclusive STEM-focused high schools. Science Education, 2017. 101(5): p. 681-715.
98. Meheust D, Le Cann P, Reboux G, Millon L, Gangneux J-P. Indoor fungal contamination: health risks and measurement methods in hospitals, homes and workplaces. Crit Rev Microbiol. 2014; 40:248-60.
99. Mendell, M. J. and K. Kumagai, Observation-based metrics for residential dampness and mold with dose-response relationships to health: A review. Indoor Air, 2017. 27(3): p. 506-517.
100. Mendell, M. J. and R. I. Adams, The challenge for microbial measurements in buildings. Indoor Air, 2019. 29(4): p. 523-526.
101. Mendell, M. J., A. G. Mirer, K. Cheung, M. Tong, and J. Douwes, Respiratory and allergic health effects of dampness, mold, and dampness-related agents: a review of the epidemiologic evidence. Environ Health Perspect, 2011. 119.
102. Misaghi I J. Influence of environment and culture media on spore morphology of Alternaria alternata. Phytopathology. 1978; 68:29.
103. Mistry J, Chuguransky S, Williams L, Qureshi M, Salazar G A, Sonnhammer E L L, et al. Pfam: The protein families database in 2021. Nucleic Acids Res. 2021; 49: D412-9.
104. Morita Y, Kikumatsu F, Higuchi Y, Katakura Y, Takegawa K. Characterization and functional analysis of ERAD-related AAA+ ATPase Cdc48 in Aspergillus oryzae. Fungal Biol. 2020; 124:801-13.
105. Mudarri D, Fisk W J. Public health and economic impact of dampness and mold. Indoor Air. 2007; 17:226-35.
106. Mudarri, D. H., Valuing the Economic Costs of Allergic Rhinitis, Acute Bronchitis, and Asthma from Exposure to Indoor Dampness and Mold in the U S. Journal of environmental and public health, 2016. 2016: p. 2386596-2386596.
107. Muyzer G, Stams A J M. The ecology and biotechnology of sulphate-reducing bacteria. Nat Rev Microbiol. 2008; 6:441-54.
108. Nastasi N, Haines S R, Xu L, da Silva H, Divjan A, Barnes M A, et al. Morphology and quantification of fungal growth in residential dust and carpets. Build Environ. 2020; 174:106774.
109. Newacheck, P. W. and N. Halfon, Prevalence, impact, and trends in childhood disability due to asthma. Arch Pediatr Adolesc Med, 2000. 154(3): p. 287-93.
110. Nowicki E M, Shroff R, Singleton J A, Renaud D E, Wallace D, Drury J, et al. Microbiota and metatranscriptome changes accompanying the onset of gingivitis. MBio [Internet]. 2018; 9.
111. Nurmagambetov, T., R. Kuwahara, and P. Garbe, The Economic Burden of Asthma in the United States, 2008-2013. Ann Am Thorac Soc, 2018. 15(3): p. 348-356.
112. Ong, I. L. H. and K. L. Yang, Recent developments in protease activity assays and sensors. Analyst, 2017. 142(11): p. 1867-1881.
113. Ottesen E A, Young C R, Eppley J M, Ryan J P, Chavez F P, Scholin C A, et al. Pattern and synchrony of gene expression among sympatric marine microbial populations. Proc Natl Acad Sci USA. 2013; 110: E488-97.
114. Ottesen E A, Young C R, Gifford S M, Eppley J M, Marin R 3rd, Schuster S C, et al. Ocean microbes. Multispecies diel transcriptional oscillations in open ocean heterotrophic bacterial assemblages. Science. 2014; 345:207-12.
115. Pasanen A-L, Juutinen T, Jantunen M J, Kalliokoski P. Occurrence and moisture requirements of microbial growth in building materials. Int Biodeterior Biodegradation. 1992; 30:273-83.
116. Paslov, L. S. and S. C. S. University, The Effect of a Piloted Middle School Pre-engineering Program on Girls' Interest and Achievement in Mathematics. 2006: Southern Connecticut State University.
117. Paslov, L. S. Impacting Middle School Girls' Interest and Achievement in Math: Research on a Piloted Pre-engineering Program. in NERA Conference Proceedings. 2016.
118. Peccia J, Haverinen-Shaughnessy U, Täubel M, Gentner D R, Shaughnessy R. Practitioner-driven research for improving the outcomes of mold inspection and remediation. Sci Total Environ. 2021; 762:144190.
119. Phillips D. Changes in conidia of Monilinia fructicola in response to incubation temperature. Phytopathology. 1982; 72:1281-3.
120. Ponikau, J. U. and D. A. Sherris, The role of airborne mold in chronic rhinosinusitis. Journal of Allergy and Clinical Immunology, 2006. 118(3): p. 762-763.
121. Ponikau, J. U., D. A. Sherris, G. M. Kephart, E. B. Kern, D. J. Congdon, C. R. Adolphson, M. J. Springett, G. J. Gleich, and H. Kita, Striking deposition of toxic eosinophil major basic protein in mucus: implications for chronic rhinosinusitis. J Allergy Clin Immunol, 2005. 116(2): p. 362-9.
122. Pounds, S. B., Estimation and control of multiple testing error rates for microarray studies. Briefings in Bioinformatics, 2006. 7(1): p. 25-36.
123. Qian, J., D. Hospodsky, N. Yamamoto, W. W. Nazaroff, and J. Peccia, Size-resolved emission rates of airborne bacteria and fungi in an occupied classroom. Indoor Air, 2012. 22(4): p. 339-51.
124. R: A language and environment for statistical computing. R Foundation for Statistical Computing [Internet]. Vienna, Austria; [cited 2024 March].
125. Renninger N, Nastasi N, Bope A, Cochran S J, Haines S R, Balasubrahmaniam N, et al. Indoor dust as a matrix for surveillance of COVID-19. mSystems [Internet]. 2021; 6.
126. Reponen T, Lockey J, Bernstein D I. Infant origins of child-hood asthma associated with specific molds. J Allergy Clin Immunol. 2012; 130.
127. Richie D L, Feng X, Hartl L, Aimanianda V, Krishnan K, Powers-Fletcher M V, et al. The virulence of the opportunistic fungal pathogen Aspergillus fumigatus requires cooperation between the endoplasmic reticulum-associated degradation pathway (ERAD) and the unfolded protein response (UPR). Virulence. 2011; 2:12-21.
128. Ringnér M. What is principal component analysis? Nat Biotechnol. 2008; 26:303-4.
129. Riquelme M, Aguirre J, Bartnicki-García S, Braus G H, Feldbrügge M, Fleig U, et al. Fungal morphogenesis, from the polarized growth of hyphae to complex reproduction and infection structures. Microbiol Mol Biol Rev [Internet]. 2018; 82.
130. Robinson M D, Oshlack A. A scaling normalization method for differential expression analysis of RNA-seq data. Genome Biol. 2010; 11: R25.
131. Rolling T, Zhai B, Frame J, Hohl T M, Taur Y. Customization of a DADA2-based pipeline for fungal internal transcribed spacer 1(ITS1) amplicon data sets. JCI Insight [Internet]. 2022; 7.
132. Sabar M A, Honda R, Haramoto E. CrAssphage as an indicator of human-fecal contamination in water environment and virus reduction in wastewater treatment. Water Res. 2022; 221:118827.
133. Satala D, Bras G, Kozik A, Rapala-Kozik M, Karkowska-Kuleta J. More than just protein degradation: The regulatory roles and moonlighting functions of extracellular proteases produced by fungi pathogenic for humans. J Fungi (Basel). 2023; 9:121.
134. Sever M, Arbes S J Jr, Vaughn B, Mehta J, Lynch J T, Mitchell H, et al. Feasibility of using subject-collected dust samples in epidemiological and clinical studies of indoor allergens. J Allergy Clin Immunol. 2005; 115: S97.
135. Shapiro S S, Wilk M B. An analysis of variance test for normality (complete samples). Biometrika. 1965; 52:591-611.
136. Shorter C, Crane J, Pierse N. Indoor visible mold and mold odour are associated with new-onset childhood wheeze in a dose dependent manner. Indoor Air. 2018; 28:6-15.
137. Soneson C, Love M I, Robinson M D. Differential analyses for RNA-seq: transcript-level estimates improve gene-level inferences. F1000Res. 2015; 4:1521.
138. Song L, Florea L. Rcorrector: efficient and accurate error correction for Illumina RNA-seq reads. Gigascience. 2015; 4:48.
139. Sonnhammer E L, Eddy S R, Durbin R. Pfam: a comprehensive database of protein domain families based on seed alignments. Proteins. 1997; 28:405-20.
140. Sothern, B. Overcoming the Barriers to Establishing Quantitative Health Guidance Values for Airborne Mold. in 2019 IAQA Annual Meeting. 2019.
141. Stachler E, Bibby K. Metagenomic evaluation of the highly abundant human gut bacteriophage CrAssphage for source tracking of human fecal pollution. Environ Sci Technol Lett. 2014; 1:405-9.
142. Steinberg G, Peñalva M A, Riquelme M, Wösten H A, Harris S D. Cell biology of hyphal growth. Microbiol Spectr [Internet]. 2017; 5.
143. Storey J D, Taylor J E, Siegmund D. Strong control, conservative point estimation and simultaneous conservative consistency of false discovery rates: A unified approach. J R Stat Soc Series B Stat Methodol. 2004; 66:187-205.
144. Story, C., Same Mold Story, in Health Indoors. 2018.
145. Verdoes, M. and S. H. Verhelst, Detection of protease activity in cells and animals. Biochim Biophys Acta, 2016. 1864(1): p. 130-42.
146. Wagner G P, Kin K, Lynch V J. Measurement of mRNA abundance using RNA-seq data: RPKM measure is inconsistent among samples. Theory Biosci. 2012; 131:281-5.
147. Wang, X., Why students choose STEM majors: Motivation, high school learning, and postsecondary context of support. American Educational Research Journal, 2013. 50(5): p. 1081-1121.
148. Wei T, Simko V, Levy M, Xie Y, Jin Y, Zemla J. Package ‘corrplot’. Statistician. 2017; 56:316-24.
149. WHO. Guidelines for Indoor Air Quality: Dampness and Mould. Copenhagen: World Health Organization; 2009.
150. Wickham H. ggplot2: elegant graphics for data analysis. Springer Nature; 2016.
151. Wolf J, Neill O, Rogers N R, Muilenberg C A, Ziska M L. Elevated atmospheric carbon dioxide concentrations amplify Alternaria alternate sporulation and total antigen production. Environ Health Perspect. 2010; 118:1223-8.
152. Young M D, Wakefield M J, Smyth G K, Oshlack A. Gene ontology analysis for RNA-seq: accounting for selection bias. Genome Biol. 2010; 11: R14.
153. Zahran, H. S., C. Bailey, and P. Garbe, Vital Signs: Asthma Prevalence, Disease Characteristics, and Self-Management Education - - - United States, 2001-2009. Morbidity and Mortality Weekly Report (MMWR), 2011. 60(17): p. 547-552.
154. Zhang Z, Reponen T, Hershey G K K. Fungal exposure and asthma: IgE and non-IgE-mediated mechanisms. Curr Allergy Asthma Rep. 2016; 16:86.
155. Zhang, S., N. Shapiro, G. Gehrke, J. Castner, Z. Liu, B. Guo, R. Prasad, J. Zhang, S. Haines, D. Kormos, P. Frey, R. Qin, and K. C. Dannemiller, Smartphone app for residential testing of formaldehyde (SmART-Form). Building and Environment, 2018.

1. A method of reducing or inhibiting microbial growth in a built environment comprising:
a) identifying microbial growth by detecting and quantifying one or more gene(s) or product(s) thereof selected from Table 2 in at least one sample collected from the built environment, wherein the one or more gene(s) and product(s) thereof are associated with a bio-process of growth or sporulation of a microbe; and
b) inhibiting or reducing microbial growth in the built environment based on results of step a).

2. The method of claim 1, wherein the one or more gene(s) or product(s) thereof are selected from ALTA7, atfB, catA, hsp30, nuo-49, rodA, wA, arp1, arp2, gel2, gna-3, mpg1, mtlD, pkaR, tpsA, velB, vosA, wetA, Canx homolog, tpiA, camA, crzA, ecm33, hex1, bimG, mdm10, cap2, catB, chsA, gpaA, nudA, rhoC, steA, candA-C, candA-N, ccg-8, dop1, flbA, fluG, laeA, nudG, sidH, chsC, grrA, hymA, or sidC.

3. The method of claim 1, wherein the one or more gene(s) and product(s) thereof are assessed using functional annotation related to fungal (mold) growth and sporulation.

4. The method of claim 1, wherein the functional annotation is Gene Ontology or GO.

5. The method of claim 1, wherein the built environment is selected from a group comprising a laboratory, a hospital, a manufacturing plant, an airport, an airplane, a school, an office, a vehicle, an apartment complex, a dormitory, a barrack, a prison, a spacecraft, or a home.

6. The method of claim 1, wherein equilibrium relative humidity (ERH) is between 30-100% in the built environment.

7. The method of claim 1, wherein the at least one sample comprises a dust sample, a surface sample, an air sample, a water sample, and/or a combination of environmental samples.

8. The method of claim 1, wherein the microbe is a bacterium and/or fungus or protozoa.

9. The method of claim 8, wherein the fungus is mold.

10. The method of claim 8, wherein the fungus is Aspergillus, Neurospora, Myxococcus, Saccharomyces, Penicillium or any other fungal taxa.

11. The method of claim 1, wherein the one or more gene(s) or product(s) thereof are measured by identifying a protein, a metabolite, a volatile organic compound, a chemical product, or a nucleic acid in the sample.

12-17. (canceled)

18. The method of claim 1, wherein the microbial growth is bacterial growth, and the one or more gene(s) is selected from a group of bacterial genes comprising rhoC or rodA.

19. The method of claim 1, wherein at least one fungal gene and at least one bacterial gene are selected.

20-23. (canceled)

24. The method of claim 1, which further comprises comparing the expression of one or more gene(s) or the quantity of product(s) thereof to a control with a threshold value, database value, normalized value, relative value, validated value, or a combination thereof.

25. The method of claim 24, wherein an increase in the expression of the one or more gene(s) or the quantity of product(s) thereof compared to the control indicates microbial growth.

26. The method of claim 24, wherein a decrease in the expression of the one or more gene(s) or the quantity of product(s) thereof compared to the control indicates a lack of microbial growth.

27. The method of claim 1, wherein any one gene is used to normalize the expression of the one or more gene(s) or the quantity of product(s) thereof.

28. The method of claim 2, wherein the ALTA7, atfB, catA, hsp30, nuo-49, rodA, wA, arp1, arp2, gel2, gna-3, mpg1, mtID, pkaR, tpsA, velB, vosA, wetA, Canx homolog, tpiA, camA, crzA, ecm33, hex1, bimG, mdm10, cap2, catB, chsA, gpaA, nudA, rhoC, steA, candA-C, candA-N, ccg-8, dop1, flbA, fluG, laeA, nudG, sidH, chsC, grrA, hymA, or sidC are found in multiple taxa in the fungal kingdom, including Aspergillus nidulans, Neurospora crassa, Myxococcus xanthus, Saccharomyces Cerevisiae and other fungal taxa.

29.-30. (canceled)

31. The method of claim 1, wherein microbial growth is inhibited or reduced by treating the built environment with a microbial growth inhibitor, at least, once daily for at least a week.

32. The method of claim 31, wherein the microbial growth inhibitor is selected from a dehumidifier, an exhaust fan, an anti-microbial compound, a hydrophobic paint, or a combination thereof.

33-68. (canceled)
